(19) United States
(12) Patent Application Publication
(10) Pub. No.: US 2008/0213763 A1
(43) Pub. Date: Sep. 4, 2008

(54) Human Protooncogene and Protein Encoded by Same, and Expression Vector Containing Same

(76) Inventors: Hyun-Kee Kim, Seoul (KR); Jin-Woo Kim, Seoul (KR)

Correspondence Address: COHEN, PONTANI, LIEBERMAN & PAVANE LLP, 551 FIFTH AVENUE, SUITE 1210, NEW YORK, NY, 10176, US

(21) Appl. No.: 11/794,272

(22) PCT Filed: Dec. 28, 2005
(86) PCT No.: PCT/KR05/04619

§ 371 Date: Jun. 26, 2007

(30) Foreign Application Priority Data
Dec. 28, 2004 (KR) 10-2004-0114310

(51) Int. Cl. C12Q 1/68; C07K 14/00; C07H 21/04; C12N 15/00; C12Q 1/00

(52) U.S. Cl. 435/6; 530/350; 536/23.5; 435/320.1; 435/4

(57) ABSTRACT

Disclosed are a novel protooncogene and a protein encoded by same. The protooncogene of the present invention is a novel gene, and may be effectively used for diagnosing the cancers, including leukemia, uterine cancer, lymphoma, colon cancer, lung cancer, skin cancer, etc., as well as producing transformed animals, etc.

### TECHNICAL FIELD

The present invention relates to human protooncogenes, proteins encoded by same, expression vectors containing same, and cells transformed by the vector.

### BACKGROUND ART

Generally, it has been known that the higher animals, including human, have approximately 30,000 genes, but only approximately 15% of the genes are expressed in each subject. Accordingly, it was found that all phenomena of life, namely generation, differentiation, homeostasis, responses to stimulus, control of cell cycle, aging and apoptosis (programmed cell death), etc. were determined depending on which genes are selected and expressed (Liang, P. and A. B. Pardee, Science 257: 967-971, 1992).

The pathological phenomena such as oncogenesis are induced by the genetic variation, resulting in changed expression of the genes. Accordingly, comparison of the gene expressions between different cells may be a basic and fundamental approach to understand various biological mechanisms.

For example, the mRNA differential display method proposed by Liang and Pardee (Liang, P. and A. B. Pardee, see the above reference) has been effectively used for searching tumor suppressor genes, genes relevant to cell cycle regulation, and transcriptional regulatory genes relevant to apoptosis, etc., and also widely employed for specifying correlations of the various genes that rise only in one cell.

Putting together the various results of oncogenesis, it has been reported that various genetic changes such as loss of specific chromosomal heterozygosity, activation of the protooncogenes, and inactivation of other tumor suppressor genes including the p53 gene was accumulated in the tumor tissues to develop human tumors (Bishop, J. M., Cell 64: 235-248, 1991; Hunter, T., Cell 64: 249-270, 1991). Also, it was reported that 10 to 30% of the cancer was activated by amplifying the protooncogenes. As a result, the activation of protooncogenes plays an important role in the etiological studies of many cancers, and therefore there have been attempts to specify the role.

Accordingly, the present inventors found that a mechanism for generating lung cancer and cervical cancer was studied in a protooncogene level, and therefore the protooncogene, named a human proliferation-inducing gene, showed a specifically increased level of expression only in the cancer cell. The protooncogene may be effectively used for diagnosing the various cancers such as leukemia, uterine cancer, lymphoma, colon cancer, lung cancer, skin cancer, etc.

### DISCLOSURE OF INVENTION

Accordingly, the present invention is designed to solve the problems of the prior art, and therefore it is an object of the present invention to provide novel protooncogenes and their fragments.

It is another object of the present invention to provide recombinant vectors containing each of the protooncogenes and their fragments; and microorganisms transformed by each of the recombinant vectors.

It is still another object of the present invention to provide proteins encoded by each of the protooncogenes; and their fragments.

It is still another object of the present invention to provide a kit for diagnosing cancer, including each of the protooncogenes or their fragments.

It is yet another object of the present invention to provide a kit for diagnosing cancer, including each of the proteins or their fragments.

In order to accomplish the above object, the present invention provides a protooncogene having a DNA sequence of SEQ ID NO: 1; or its fragments.

According to the another object, the present invention provides a protein having an amino acid sequence of SEQ ID NO: 2; or its fragments.

Also, the present invention provides a protooncogene having a DNA sequence of SEQ ID NO: 5; or its fragments.

The present invention provides a protein having an amino acid sequence of SEQ ID NO: 6; or its fragments.

Also, the present invention provides a protooncogene having a DNA sequence of SEQ ID NO: 9; or its fragments.

The present invention provides a protein having an amino acid sequence of SEQ ID NO: 10; or its fragments.

Also, the present invention provides a protooncogene having a DNA sequence of SEQ ID NO: 13; or its fragments.

The present invention provides a protein having an amino acid sequence of SEQ ID NO: 14; or its fragments.

Also, the present invention provides a protooncogene having a DNA sequence of SEQ ID NO: 17; or its fragments.

The present invention provides a protein having an amino acid sequence of SEQ ID NO: 18; or its fragments.

Also, the present invention provides a protooncogene having a DNA sequence of SEQ ID NO: 21; or its fragments.

The present invention provides a protein having an amino acid sequence of SEQ ID NO: 22; or its fragments.

Also, the present invention provides a protooncogene having a DNA sequence of SEQ ID NO: 25; or its fragments.

The present invention provides a protein having an amino acid sequence of SEQ ID NO: 26; or its fragments.

Also, the present invention provides a protooncogene having a DNA sequence of SEQ ID NO: 29; or its fragments.

The present invention provides a protein having an amino acid sequence of SEQ ID NO: 30; or its fragments.

Also, the present invention provides a protooncogene having a DNA sequence of SEQ ID NO: 33; or its fragments.

The present invention provides a protein having an amino acid sequence of SEQ ID NO: 34; or its fragments.

According to the said object, the present invention provides a protooncogene having a DNA sequence of SEQ ID NO: 37; or its fragments.

The present invention provides a protein having an amino acid sequence of SEQ ID NO: 38; or its fragments.

Also, the present invention provides a protooncogene having a DNA sequence of SEQ ID NO: 39; or its fragments.

The present invention provides a protein having an amino acid sequence of SEQ ID NO: 40; or its fragments.

According to the another object, the present invention provides kits for diagnosing cancer, including each of the protooncogenes or their fragments.

According to the still another object, the present invention provides kits for diagnosing cancer, including each of the protooncoproteins or their fragments.

### BRIEF DESCRIPTION OF THE DRAWINGS

These and other features, aspects, and advantages of preferred embodiments of the present invention will be more fully described in the following detailed description, taken accompanying drawings. In the drawings:

FIG. 1 is a gel diagram showing a result of the differential display reverse transcription-polymerase chain reaction (DDRT-PCR) to determine whether or not an L699 gene is expressed in a normal lung tissue, a left lung cancer tissue, a metastatic lung cancer tissue metastasized from the left lung to the right lung, and an A549 lung cancer cell;

FIG. 2 is a gel diagram showing a result of the differential display reverse transcription-polymerase chain reaction (DDRT-PCR) to determine whether or not a CA325 DNA fragment is expressed in a normal exocervical tissue, a cervical tumor tissue, a metastatic lymph node tumor tissue and a CUMC-6 cancer cell;

FIG. 3 is a gel diagram showing a result of the differential display reverse transcription-polymerase chain reaction (DDRT-PCR) to determine whether or not a CA273 DNA fragment is expressed in a normal exocervical tissue, a cervical tumor tissue, a metastatic lymph node tumor tissue and a CUMC-6 cancer cell;

FIG. 4 is a gel diagram showing a result of the differential display reverse transcription-polymerase chain reaction (DDRT-PCR) to determine whether or not an L667 DNA fragment is expressed in a normal lung tissue, a left lung cancer tissue, a metastatic lung cancer tissue metastasized from the left lung to the right lung, and an A549 lung cancer cell;

FIG. 5 is a gel diagram showing a result of the differential display reverse transcription-polymerase chain reaction (DDRT-PCR) to determine whether or not an L668 DNA fragment is expressed in a normal lung tissue, a left lung cancer tissue, a metastatic lung cancer tissue metastasized from the left lung to the right lung, and an A549 lung cancer cell;

FIG. 6 is a gel diagram showing a result of the differential display reverse transcription-polymerase chain reaction (DDRT-PCR) to determine whether or not an L211 DNA fragment is expressed in a normal lung tissue, a left lung cancer tissue, a metastatic lung cancer tissue metastasized from the left lung to the right lung, and an A549 lung cancer cell;

FIG. 7 is a gel diagram showing a result of the differential display reverse transcription-polymerase chain reaction (DDRT-PCR) to determine whether or not an L722 DNA fragment is expressed in a normal lung tissue, a left lung cancer tissue, a metastatic lung cancer tissue metastasized from the left lung to the right lung, and an A549 lung cancer cell;

FIG. 8 is a gel diagram showing a result of the differential display reverse transcription-polymerase chain reaction (DDRT-PCR) to determine whether or not an L752 DNA fragment is expressed in a normal lung tissue, a left lung cancer tissue, a metastatic lung cancer tissue metastasized from the left lung to the right lung, and an A549 lung cancer cell;

FIG. 9 is a gel diagram showing a result of the differential display reverse transcription-polymerase chain reaction (DDRT-PCR) to determine whether or not an L1003 DNA fragment is expressed in a normal lung tissue, a left lung cancer tissue, a metastatic lung cancer tissue metastasized from the left lung to the right lung, and an A549 lung cancer cell;

FIG. 10 is a gel diagram showing a result of the differential display reverse transcription-polymerase chain reaction (DDRT-PCR) to determine whether or not an HP90-8115 DNA fragment is expressed in a normal exocervical tissue, a cervical tumor tissue, a metastatic lymph node tumor tissue and a CUMC-6 cancer cell;

FIG. 11(a) is a gel diagram showing a northern blotting result to determine whether or not the PIG5 protooncogene is expressed in the normal lung tissue, the left lung cancer tissue, the metastatic lung cancer tissue metastasized from the left lung to the right lung, and the A549, NCI-H2009 and NCI-H441 lung cancer cell lines, and FIG. 11(b) is a diagram showing a northern blotting result obtained by hybridizing the same sample as in FIG. 11(a) with β-actin probe;

FIG. 12(a) is a gel diagram showing a northern blotting result to determine whether or not the PIG6 protooncogene of the present invention is expressed in the normal exocervical tissue, the uterine cancer tissue, the metastatic cervical lymph node tissue and the cervical cancer cell line, and FIG. 12(b) is a diagram showing a northern blotting result obtained by hybridizing the same sample as in FIG. 12(a) with β-actin probe;

FIG. 13(a) is a gel diagram showing a northern blotting result to determine whether or not the PIG7 protooncogene of the present invention is expressed in the normal exocervical tissue, the uterine cancer tissue, the metastatic cervical lymph node tissue and the cervical cancer cell line, and FIG. 13(b) is a diagram showing a northern blotting result obtained by hybridizing the same sample as in FIG. 13(a) with β-actin probe;

FIG. 14(a) is a gel diagram showing a northern blotting result to determine whether or not the PIG11 protooncogene is expressed in the normal lung tissue, the left lung cancer tissue, the metastatic lung cancer tissue metastasized from the left lung to the right lung, and the A549, NCI-H2009 and NCI-H441 lung cancer cell lines, and FIG. 14(b) is a diagram showing a northern blotting result obtained by hybridizing the same sample as in FIG. 14(a) with β-actin probe;

FIG. 15(a) is a gel diagram showing a northern blotting result to determine whether or not the PIG16 protooncogene is expressed in the normal lung tissue, the left lung cancer tissue, the metastatic lung cancer tissue metastasized from the left lung to the right lung, and the A549, NCI-H2009 and NCI-H441 lung cancer cell lines, and FIG. 15(b) is a diagram showing a northern blotting result obtained by hybridizing the same sample as in FIG. 15(a) with β-actin probe;

FIG. 16(a) is a gel diagram showing a northern blotting result to determine whether or not the PIG17 protooncogene is expressed in the normal lung tissue, the left lung cancer tissue, the metastatic lung cancer tissue metastasized from the left lung to the right lung, and the A549, NCI-H2009 and NCI-H441 lung cancer cell lines, and FIG. 16(b) is a diagram showing a northern blotting result obtained by hybridizing the same sample as in FIG. 16(a) with β-actin probe;

FIG. 17(a) is a gel diagram showing a northern blotting result to determine whether or not the PIG19 protooncogene is expressed in the normal lung tissue, the left lung cancer tissue, the metastatic lung cancer tissue metastasized from the left lung to the right lung, and the A549, NCI-H2009 and NCI-H441 lung cancer cell lines, and FIG. 17(b) is a diagram showing a northern blotting result obtained by hybridizing the same sample as in FIG. 17(a) with β-actin probe;

FIG. 18(a) is a gel diagram showing a northern blotting result to determine whether or not the PIG20 protooncogene is expressed in the normal lung tissue, the left lung cancer tissue, the metastatic lung cancer tissue metastasized from the left lung to the right lung, and the A549, NCI-H2009 and NCI-H441 lung cancer cell lines, and FIG. 18(b) is a diagram showing a northern blotting result obtained by hybridizing the same sample as in FIG. 18(a) with β-actin probe;

FIG. 19(a) is a gel diagram showing a northern blotting result to determine whether or not the PIG21 protooncogene is expressed in the normal lung tissue, the left lung cancer tissue, the metastatic lung cancer tissue metastasized from the left lung to the right lung, and the A549, NCI-H2009 and NCI-H441 lung cancer cell lines, and FIG. 19(b) is a diagram showing a northern blotting result obtained by hybridizing the same sample as in FIG. 19(a) with β-actin probe;

FIG. 20 is a gel diagram showing a northern blotting result to determine whether or not the HCCRBP2 protooncogene is expressed in the promyelocyte leukemia cell line HL-60, the uterine cancer cell line HeLa, the chronic myelogenous leukemia cell line K-562, the lymphoblastic leukaemia cell line MOLT-4, the Burkitt lymphoma cell line Raji, the colon cancer cell line SW480, the lung cancer cell line A549 and the melanoma skin cancer cell line G361;

FIG. 21(a) is a gel diagram showing a northern blotting result to determine whether or not the TRG2 protooncogene of the present invention is expressed in the normal exocervical tissue, the uterine cancer tissue, the metastatic cervical lymph node tissue and the cervical cancer cell line, and FIG. 21(b) is a diagram showing a northern blotting result obtained by hybridizing the same sample as in FIG. 21(a) with β-actin probe;

FIG. 22(a) is a diagram showing a northern blotting result to determine whether or not the PIG5 protooncogene is expressed in a normal human 12-lane multiple tissues, and FIG. 22(b) is a diagram showing a northern blotting result obtained by hybridizing the same sample as in FIG. 22(a) with β-actin probe;

FIG. 23 is a diagram showing a northern blotting result to determine whether or not the PIG6 protooncogene of the present invention is expressed in a normal human 12-lane multiple tissues;

FIG. 24 is a diagram showing a northern blotting result obtained by hybridizing the same sample as in FIG. 23 with β-actin probe;

FIG. 25 is a diagram showing a northern blotting result to determine whether or not the PIG7 protooncogene of the present invention is expressed in a normal human 12-lane multiple tissues;

FIG. 26 is a diagram showing a northern blotting result obtained by hybridizing the same sample as in FIG. 25 with β-actin probe;

FIG. 27(a) is a diagram showing a northern blotting result to determine whether or not the PIG11 protooncogene is expressed in a normal human 12-lane multiple tissues, and FIG. 27(b) is a diagram showing a northern blotting result obtained by hybridizing the same sample as in FIG. 27(a) with β-actin probe;

FIG. 28(a) is a diagram showing a northern blotting result to determine whether or not the PIG16 protooncogene is expressed in a normal human 12-lane multiple tissues, and FIG. 28(b) is a diagram showing a northern blotting result obtained by hybridizing the same sample as in FIG. 28(a) with β-actin probe;

FIG. 29(a) is a diagram showing a northern blotting result to determine whether or not the PIG17 protooncogene is expressed in a normal human 12-lane multiple tissues, and FIG. 29(b) is a diagram showing a northern blotting result obtained by hybridizing the same sample as in FIG. 29(a) with β-actin probe;

FIG. 30(a) is a diagram showing a northern blotting result to determine whether or not the PIG19 protooncogene is expressed in a normal human 12-lane multiple tissues, and FIG. 30(b) is a diagram showing a northern blotting result obtained by hybridizing the same sample as in FIG. 30(a) with β-actin probe;

FIG. 31(a) is a diagram showing a northern blotting result to determine whether or not the PIG20 protooncogene is expressed in a normal human 12-lane multiple tissues, and FIG. 31(b) is a diagram showing a northern blotting result obtained by hybridizing the same sample as in FIG. 31(a) with β-actin probe;

FIG. 32(a) is a diagram showing a northern blotting result to determine whether or not the PIG21 protooncogene is expressed in a normal human 12-lane multiple tissues, and FIG. 32(b) is a diagram showing a northern blotting result obtained by hybridizing the same sample as in FIG. 32(a) with β-actin probe;

FIG. 33 is a diagram showing a northern blotting result to determine whether or not the HCCRBP2 protooncogene is expressed in a normal human 12-lane multiple tissues, for example brain, heart, skeletal muscles, large intestines, thymus, spleen, kidney, liver, small intestines, placenta, lung and peripheral blood leukocyte;

FIG. 34 is a diagram showing a northern blotting result to determine whether or not the TRG2 protooncogene of the present invention is expressed in a normal human 12-lane multiple tissues;

FIG. 35 is a diagram showing a result obtained by hybridizing the same sample as in FIG. 34 with β-actin probe;

FIG. 36(a) is a diagram showing a northern blotting result to determine whether or not the PIG5 protooncogene is expressed in human cancer cell lines, and FIG. 36(b) is a diagram showing a result obtained by hybridizing the same sample as in FIG. 36(a) with β-actin probe;

FIG. 37 is a diagram showing a northern blotting result to determine whether or not the PIG6 protooncogene of the present invention is expressed in human cancer cell lines;

FIG. 38 is a diagram showing a result obtained by hybridizing the same sample as in FIG. 37 with β-actin probe;

FIG. 39 is a diagram showing a northern blotting result to determine whether or not the PIG7 protooncogene of the present invention is expressed in human cancer cell lines;

FIG. 40 is a diagram showing a result obtained by hybridizing the same sample as in FIG. 39 with β-actin probe;

FIG. 41(a) is a diagram showing a northern blotting result to determine whether or not the PIG11 protooncogene is expressed in human cancer cell lines, and FIG. 41(b) is a diagram showing a result obtained by hybridizing the same sample as in FIG. 41(a) with β-actin probe;

FIG. 42(a) is a diagram showing a northern blotting result to determine whether or not the PIG16 protooncogene is expressed in human cancer cell lines, and FIG. 42(b) is a diagram showing a result obtained by hybridizing the same sample as in FIG. 42(a) with β-actin probe;

FIG. 43(a) is a diagram showing a northern blotting result to determine whether or not the PIG17 protooncogene is expressed in human cancer cell lines, and FIG. 43(b) is a diagram showing a result obtained by hybridizing the same sample as in FIG. 43(a) with β-actin probe;

FIG. 44(a) is a diagram showing a northern blotting result to determine whether or not the PIG19 protooncogene is expressed in human cancer cell lines, and FIG. 44(b) is a diagram showing a result obtained by hybridizing the same sample as in FIG. 44(a) with β-actin probe;

FIG. 45(a) is a diagram showing a northern blotting result to determine whether or not the PIG20 protooncogene is expressed in human cancer cell lines, and FIG. 45(b) is a diagram showing a result obtained by hybridizing the same sample as in FIG. 45(a) with β-actin probe;

FIG. 46(a) is a diagram showing a northern blotting result to determine whether or not the PIG21 protooncogene is expressed in human cancer cell lines, and FIG. 46(b) is a diagram showing a result obtained by hybridizing the same sample as in FIG. 46(a) with β-actin probe;

FIG. 47 is a diagram showing a northern blotting result to determine whether or not the HCCRBP2 protooncogene is expressed in uterine cancer tissues (top), and a northern blotting result obtained by hybridizing the same sample as in the top of FIG. 47 with β-actin probe (bottom);

FIG. 48 is a diagram showing a northern blotting result to determine whether or not the HCCRBP2 protooncogene is expressed in colon cancer tissues (top), and a northern blotting result obtained by hybridizing the same sample as in the top of FIG. 48 with β-actin probe (bottom);

FIG. 49 is a diagram showing a northern blotting result to determine whether or not the HCCRBP2 protooncogene is expressed in leukemia tissues (top), and a northern blotting result obtained by hybridizing the same sample as in the top of FIG. 49 with β-actin probe (bottom);

FIG. 50 is a diagram showing a northern blotting result to determine whether or not the TRG2 protooncogene of the present invention is expressed in human cancer cell lines;

FIG. 51 is a diagram showing a result obtained by hybridizing the same sample as in FIG. 50 with β-actin probe;

FIGS. 52 to 62 are diagrams showing results of sodium dodecyl sulfate-polyacrylamide gel electrophoresis (SDS-PAGE) to determine sizes of the proteins expressed before and after L-arabinose induction after the PIG5, PIG6, PIG7, PIG11, PIG16, PIG17, PIG19, PIG20 and PIG21 protooncogenes of the present invention, the HCCRBP2 protooncogene, and the TRG2 protooncogene of the present invention are transformed into Escherichia coli, respectively.

### BEST MODES FOR CARRYING OUT THE INVENTION

Hereinafter, preferred embodiments of the present invention will be described in detail referring to the accompanying drawings.

1. PIG 5

The protooncogene, human proliferation-inducing gene 5 (PIG5), of the present invention (hereinafter, referred to as PIG5 protooncogene) has a 1009-bp full-length DNA sequence set forth in SEQ ID NO: 1.

In the DNA sequence of SEQ ID NO: 1, the open reading frame corresponding to nucleotide sequence positions from 9 to 746 (744-746: a stop codon) is a full-length protein coding region, and an amino acid sequence derived from the protein coding region is set forth in SEQ ID NO: 2 and contains 245 amino acids (hereinafter, referred to as “PIG5 protein”).

The DNA sequence of SEQ ID NO: 1 has been deposited with Accession No. AY236486 into the GenBank database of U.S. National Institutes of Health (NIH) (Publication Date: Dec. 31, 2004), and the DNA sequencing result revealed that some of its DNA sequence was similar to those of the Homo sapiens cDNA FLJ12453 fis, clone NT2RM1000430, moderately similar to Homo sapiens erythroblast macrophage protein EMP mRNA gene and the Homo sapiens macrophage erythroblast attacher gene, deposited with Accession No. AK022515 and BC006470 into the database, respectively.

A protein expressed from the protooncogene PIG5 of the present invention contains 245 amino acids and has an amino acid sequence set forth in SEQ ID NO: 2 and a molecular weight of approximately 23 kDa.

2. PIG 6

The protooncogene, human proliferation-inducing gene 6 (PIG6), of the present invention (hereinafter, referred to as PIG6 protooncogene) has a 2,964-bp full-length DNA sequence set forth in SEQ ID NO: 5.

In the DNA sequence of SEQ ID NO: 5, the open reading frame corresponding to nucleotide sequence positions from 293 to 2302 (2300-2302: a stop codon) is a full-length protein coding region, and an amino acid sequence derived from the protein coding region is set forth in SEQ ID NO: 6 and contains 669 amino acids (hereinafter, referred to as “PIG6 protein”).

The DNA sequence of SEQ ID NO: 5 has been deposited with Accession No. AY236487 into the GenBank database of U.S. National Institutes of Health (NIH) (Publication Date: Dec. 31, 2004), and the DNA sequencing result revealed that its DNA sequence was similar to those of the Homo sapiens HLC-6 mRNA gene and the Homo sapiens sperm associated antigen 9 (SPAG9), transcript variant gene, deposited with Accession No. AF542172 and NM_{—}003971 into the database, respectively.

A protein expressed from the protooncogene of the present invention contains 669 amino acids and has an amino acid sequence set forth in SEQ ID NO: 6 and a molecular weight of approximately 72 kDa.

3. PIG 7

The protooncogene, human proliferation-inducing gene 7 (PIG7), of the present invention (hereinafter, referred to as PIG7 protooncogene) has a 4,301-bp full-length DNA sequence set forth in SEQ ID NO: 9.

In the DNA sequence of SEQ ID NO: 9, the open reading frame corresponding to nucleotide sequence positions from 3536 to 3792 (3790-3792: a stop codon) is a full-length protein coding region, and an amino acid sequence derived from the protein coding region is set forth in SEQ ID NO: 10 and contains 78 amino acids (hereinafter, referred to as “PIG7 protein”).

The DNA sequence of SEQ ID NO: 9 has been deposited with Accession No. AY236488 into the GenBank database of U.S. National Institutes of Health (NIH) (Publication Date: Dec. 31, 2004), and the DNA sequencing result revealed that some of its DNA sequence was similar to those of the Homo sapiens chromosome 18, clone RP11-54G14 gene deposited with Accession No. AC116447 into the database.

A protein expressed from the protooncogene of the present invention contains 78 amino acids and has an amino acid sequence set forth in SEQ ID NO: 10 and a molecular weight of approximately 9 kDa.

4. PIG11

The protooncogene, human proliferation-inducing gene 11 (PIG11), of the present invention (hereinafter, referred to as PIG11 protooncogene) has a 1038-bp full-length DNA sequence set forth in SEQ ID NO: 13.

In the DNA sequence of SEQ ID NO: 13, the open reading frame corresponding to nucleotide sequence positions from 50 to 931 (929-931: a stop codon) is a full-length protein coding region, and an amino acid sequence derived from the protein coding region is set forth in SEQ ID NO: 14 and contains 293 amino acids (hereinafter, referred to as “PIG11 protein”).

The DNA sequence of SEQ ID NO: 13 has been deposited with Accession No. AY258284 into the GenBank database of U.S. National Institutes of Health (NIH) (Publication Date: Dec. 31, 2004), and the DNA sequencing result revealed that its DNA sequence was identical with that of the Homo sapiens N-methylpurine-DNA glycosylase gene deposited with Accession No. BC014991 into the database. Contrary to its functions as reported previously, it was however found from this study result that the PIG11 protooncogene was highly expressed in various human tumors including the lung cancer, while its expression was very low in various normal tissues.

A protein expressed from the protooncogene PIG11 of the present invention contains 293 amino acids and has an amino acid sequence set forth in SEQ ID NO: 14 and a molecular weight of approximately 32 kDa.

5. PIG16

The protooncogene, human proliferation-inducing gene 16 (PIG16), of the present invention (hereinafter, referred to as PIG16 protooncogene) has a 1682-bp full-length DNA sequence set forth in SEQ ID NO: 17.

In the DNA sequence of SEQ ID NO: 17, the open reading frame corresponding to nucleotide sequence positions from 696 to 1577 (1575-1577: a stop codon) is a full-length protein coding region, and an amino acid sequence derived from the protein coding region is set forth in SEQ ID NO: 18 and contains 293 amino acids (hereinafter, referred to as “PIG16 protein”).

The DNA sequence of SEQ ID NO: 17 has been deposited with Accession No. AY305873 into the GenBank database of U.S. National Institutes of Health (NIH) (Publication Date: Dec. 31, 2004), and the DNA sequencing result revealed that some of its DNA sequence was similar to that of the Homo sapiens N-methylpurine-DNA glycosylase gene deposited with Accession No. BC014991 into the database.

A protein expressed from the protooncogene PIG16 of the present invention contains 293 amino acids and has an amino acid sequence set forth in SEQ ID NO: 18 and a molecular weight of approximately 32 kDa.

6. PIG17

The protooncogene, human proliferation-inducing gene 17 (PIG17), of the present invention (hereinafter, referred to as PIG17 protooncogene) has a 626-bp full-length DNA sequence set forth in SEQ ID NO: 21.

In the DNA sequence of SEQ ID NO: 21, the open reading frame corresponding to nucleotide sequence positions from 59 to 610 (608-610: a stop codon) is a full-length protein coding region, and an amino acid sequence derived from the protein coding region is set forth in SEQ ID NO: 22 and contains 183 amino acids (hereinafter, referred to as “PIG17 protein”).

The DNA sequence of SEQ ID NO: 21 has been deposited with Accession No. AY336092 into the GenBank database of U.S. National Institutes of Health (NIH) (Publication Date: Dec. 31, 2004), and the DNA sequencing result revealed that its DNA sequence was identical with those of the Homo sapiens hypothetical protein LOC51234 (LOC51234) gene, the Pongo pygmaeus mRNA; cDNA DKFZp468K0411 (from clone DKFZp468K0411) gene, and the Homo sapiens HSPC184 mRNA gene, deposited with Accession No. NM_{—}016454, CR858446 and AF151018 into the database, respectively. From this study result, it was found that the PIG17 protooncogene was highly expressed in various human tumors including the lung cancer, while its expression was very low in various normal tissues.

A protein expressed from the protooncogene PIG17 of the present invention contains 183 amino acids and has an amino acid sequence set forth in SEQ ID NO: 22 and a molecular weight of approximately 20 kDa.

7. PIG19

The protooncogene, human proliferation-inducing gene 19 (PIG19), of the present invention (hereinafter, referred to as PIG19 protooncogene) has a 1031-bp full-length DNA sequence set forth in SEQ ID NO: 25.

In the DNA sequence of SEQ ID NO: 25, the open reading frame corresponding to nucleotide sequence positions from 32 to 1030 (1028-1030: a stop codon) is a full-length protein coding region, and an amino acid sequence derived from the protein coding region is set forth in SEQ ID NO: 26 and contains 332 amino acids (hereinafter, referred to as “PIG19 protein”).

The DNA sequence of SEQ ID NO: 25 has been deposited with Accession No. AY423727 into the GenBank database of U.S. National Institutes of Health (NIH) (Publication Date: Dec. 31, 2004), and the DNA sequencing result revealed that its DNA sequence was identical with those of the Homo sapiens lactate dehydrogenase A (LDHA) gene deposited with Accession No. NM_{—}005566 into the database. Contrary to its functions as reported previously, it was however found from this study result that the PIG19 protooncogene was highly expressed in various human tumors including the lung cancer, while its expression was very low in various normal tissues.

A protein expressed from the protooncogene PIG19 of the present invention contains 332 amino acids and has an amino acid sequence set forth in SEQ ID NO: 26 and a molecular weight of approximately 37 kDa.

8. PIG20

The protooncogene, human proliferation-inducing gene 20 (PIG20), of the present invention (hereinafter, referred to as PIG20 protooncogene) has a 526-bp full-length DNA sequence set forth in SEQ ID NO: 29.

In the DNA sequence of SEQ ID NO: 29, the open reading frame corresponding to nucleotide sequence positions from 1 to 498 (496-498: a stop codon) is a full-length protein coding region, and an amino acid sequence derived from the protein coding region is set forth in SEQ ID NO: 30 and contains 165 amino acids (hereinafter, referred to as “PIG20 protein”).

The DNA sequence of SEQ ID NO: 29 has been deposited with Accession No. AY423728 into the GenBank database of U.S. National Institutes of Health (NIH) (Publication Date: Dec. 31, 2004), and the DNA sequencing result revealed that its DNA sequence was identical with that of the Homo sapiens reticulocalbin 1, EF-hand calcium binding domain (RCN1) gene deposited with Accession No. NM_{—}002901 into the database. It has been reported that the reticulocalbin 1 gene is a calcium-binding protein present in endoplasmic reticulum (Ozawa, M. and Muramatsu, T. J. Biol. Chem., 268, 699-705 (1993); Ozawa, M. J. Biochem (Tokyo), 117, 1113-1119 (1995)). Contrary to its functions as reported previously, it was however found from this study result that the PIG20 protooncogene was very highly expressed in various human tumors including the lung cancer, while its expression was very low in various normal tissues.

However, because of degeneracy of codons, or considering preference of codons for living organisms to express the protooncogene, the protooncogene of the present invention may be variously modified in coding regions without changing an amino acid sequence of the protooncoprotein expressed from the coding region, and also be variously modified or changed in a region except the coding region within a range that does not affect the gene expression. Such a modified gene is also included in the scope of the present invention. Accordingly, the present invention also includes the protooncogene of SEQ ID NO: 29, a polynucleotide having substantially the same DNA sequence as the protooncogene of SEQ ID NO: 29; and fragments of the genes. The term “substantially the same polynucleotide” means a polynucleotide having DNA sequence homology of at least 80%, preferably at least 90%, and the most preferably at least 95%.

A protein expressed from the protooncogene PIG20 of the present invention contains 165 amino acids and has an amino acid sequence set forth in SEQ ID NO: 30 and a molecular weight of approximately 19 kDa.

However, one or more amino acids may be also substituted, added or deleted in the amino acid sequence of the protein within a range that does not affect functions of the protein, and only some portion of the protein may be used depending on its usage. Such a modified amino acid sequence is also included in the scope of the present invention. Accordingly, the present invention also includes a polypeptide having substantially the same amino acid sequence as the oncogenic protein; and fragments of the protein. The term “substantially the same polypeptide” means a polypeptide having sequence homology of at least 80%, preferably at least 90%, and the most preferably at least 95%.

9. PIG21

The protooncogene, human proliferation-inducing gene 21 (PIG21), of the present invention (hereinafter, referred to as PIG21 protooncogene) has a 965-bp full-length DNA sequence set forth in SEQ ID NO: 33.

In the DNA sequence of SEQ ID NO: 33, the open reading frame corresponding to nucleotide sequence positions from 146 to 961 (959-961: a stop codon) is a full-length protein coding region, and an amino acid sequence derived from the protein coding region is set forth in SEQ ID NO: 34 and contains 271 amino acids (hereinafter, referred to as “PIG21 protein”).

The DNA sequence of SEQ ID NO: 33 has been deposited with Accession No. AY336089 into the GenBank database of U.S. National Institutes of Health (NIH) (Publication Date: Dec. 31, 2004), and the DNA sequencing result revealed that some of its DNA sequence was similar to those of the full-length cDNA clone CS0DA008YE03 of Neuroblastoma of Homo sapiens (human) gene, the full-length cDNA clone CS0DI031YI19 of Placenta Cot 25-normalized of Homo sapiens (human) gene, and the Homo sapiens guanine nucleotide binding protein (G protein), beta polypeptide 2-like 1, mRNA (cDNA clone IMAGE:4705256) gene, deposited with Accession No. CR625157, CR616147 and BC035460 into the database, respectively.

A protein expressed from the protooncogene PIG21 of the present invention contains 271 amino acids and has an amino acid sequence set forth in SEQ ID NO: 34 and a molecular weight of approximately 30 kDa.

10. HCCRBP2

The protooncogene of the present invention is named HCCRBP2 which has a 626-bp full-length DNA sequence set forth in SEQ ID NO: 37, and has a property that it binds to a human cervical cancer 1 protooncogene (hereinafter, referred to as HCCR-1 protooncogene) as described in Korean Patent Application No. 2000-16757 filed by this applicant.

The DNA sequence of SEQ ID NO: 37 has been deposited with Accession No. AY323819 into the GenBank database of U.S. National Institutes of Health (NIH) (Publication Date: Dec. 31, 2004), and the DNA sequencing result revealed that its DNA sequence was similar to those of the Homo sapiens tumor protein D52-like 2 (TPD52L2), transcript variant 5 gene, and the Homo sapiens tumor protein D52-like 2, transcript variant 5, mRNA (cDNA clone MGC:5064 IMAGE:3446037) gene, deposited with Accession No. NM_{—}003288 and BC006804 into the database, respectively.

In the DNA sequence of SEQ ID NO: 37, the open reading frame corresponding to nucleotide sequence positions from 9 to 356 is a full-length protein coding region, and an amino acid sequence derived from the protein coding region is set forth in SEQ ID NO: 38 and contains 115 amino acids (hereinafter, referred to as “HCCRBP2 protein”).

A protein expressed from the protooncogene of the present invention contains 115 amino acids and has an amino acid sequence set forth in SEQ ID NO: 38 and a molecular weight of approximately 12 kDa. The protein that binds to a protein encoded by the HCCR-1 protooncogene is referred to as “HCCRBP2 (HCCR-binding protein 2)” in the present invention.

11. TRG2

The protooncogene, human transformation-related gene 2 (TRG2), of the present invention (hereinafter, referred to as TRG2 protooncogene) has a 2,302-bp full-length DNA sequence set forth in SEQ ID NO: 39.

In the DNA sequence of SEQ ID NO: 39, the open reading frame corresponding to nucleotide sequence positions from 747 to 2066 (2064-2066: a stop codon) is a full-length protein coding region, and an amino acid sequence derived from the protein coding region is set forth in SEQ ID NO: 40 and contains 439 amino acids (hereinafter, referred to as “TRG2 protein”).

The DNA sequence of SEQ ID NO: 39 has been deposited with Accession No. AY170823 into the GenBank database of U.S. National Institutes of Health (NIH) (Publication Date: Dec. 30, 2004), and the DNA sequencing result revealed that its DNA sequence was similar to those of the Homo sapiens cDNA: FLJ22058 fis, clone HEP10089, highly similar to HUMRANBP2 RanBP2 (Ran-binding protein 2) gene, the Homo sapiens nucleoporin (NUP358) gene, and the Human mRNA for RanBP2 (Ran-binding protein 2) gene, deposited with Accession No. AK025711, L41840 and D42063 into the database, respectively.

A protein expressed from the protooncogene of the present invention contains 439 amino acids and has an amino acid sequence set forth in SEQ ID NO: 40 and a molecular weight of approximately 48 kDa.

Meanwhile, because of degeneracy of codons, or considering preference of codons for living organisms to express the genes, the protooncogene of the present invention may be variously modified in coding regions without changing an amino acid sequence of the protein expressed from the coding region, and also be variously modified or changed in a region except the coding region within a range that does not affect the gene expression. Such a modified gene is also included in the scope of the present invention. Accordingly, the present invention also includes a polynucleotide having substantially the same DNA sequence as the protooncogene of SEQ ID NO: 39; and fragments of the protooncogene. The term “substantially the same polynucleotide” is referred to as DNA encoding the same translated protein product as the protein of the present invention, and means a polynucleotide having DNA sequence homology of at least 80%, preferably at least 90%, and the most preferably at least 95%.

Also, one or more amino acids may be substituted, added or deleted in the amino acid sequence of the protein within a range that does not affect functions of the protein, and only some portion of the protein may be used depending on its usage. Such a modified amino acid sequence is also included in the scope of the present invention. Accordingly, the present invention also includes a polypeptide having substantially the same amino acid sequence as the oncogenic protein; and fragments of the protein. The term “substantially the same polypeptide” means a polypeptide having sequence homology of at least 80%, preferably at least 90%, and the most preferably at least 95%.

The protooncogenes and proteins of the present invention may be separated from human cancer tissues, or also be synthesized according to the known methods for synthesizing DNA or peptide. Also, the genes prepared thus may be inserted into a vector for expression in microorganisms, already known in the art, to obtain an expression vector, and then the expression vector may be introduced into suitable host cells, for example Escherichia coli, yeast cells, etc. DNA of each of the genes of the present invention may be replicated in a large quantity or its protein may be produced in a commercial quantity in such a transformed host.

Upon constructing the expression vector, expression regulatory sequences such as a promoter and a terminator, autonomously replicating sequences, secretion signals, etc. may be suitably selected and combined depending on kinds of the host cells that produce the protooncogenes or the proteins.

The genes of the present invention are proved to be strong oncogenes capable of developing the lung cancer since it was revealed the gene was hardly expressed in a normal lung tissue, but overexpressed in a lung cancer tissue, a metastatic lung cancer tissue and a lung cancer cell line in the analysis method such as a northern blotting, etc. Also, the genes are proved to be cancer metastasis-related genes capable of inducing cancer metastasis, considering that its expression is increased in the metastasized lung cancer tissues. In addition to the epithelial tissue such as the lung cancer, the protooncogenes of the present invention are highly expressed in other cancerous tumor tissues such as leukemia, uterine cancer, lymphoma, colon cancer, lung cancer, skin cancer, etc. Accordingly, the protooncogenes of the present invention are considered to be common oncogenes in the various oncogenesis, and may be effectively used for diagnosing the various cancers and producing the transformed animals.

For example, A method for diagnosing the cancer using the protooncogenes includes a step of determining whether or not a subject has the protooncogenes of the present invention by detecting the protooncogenes in the various methods known in the art after all or some of the protooncogenes are used as proves and hybridized with nucleic acid extracted from the subject's body fluids. It can be easily confirmed that the genes are present in the tissue samples by using the probes labeled with a radioactive isotope, an enzyme, etc. Accordingly, the present invention provides kits for diagnosing the cancer containing all or some of the protooncogenes.

The transformed animals may be obtained by introducing the protooncogenes of the present invention into mammals, for example rodents such as a rat, and the protooncogenes are preferably introduced at the fertilized egg stage prior to at least 8-cell stage. The transformed animals prepared thus may be effectively used for searching carcinogenic substances or anticancer substances such as antioxidants.

The proteins derived from the protooncogenes of the present invention may be effectively used for producing antibodies as a diagnostic tool. The antibodies of the present invention may be produced as the monoclonal or polyclonal antibodies according to the conventional methods known in the art using the proteins expressed from the protooncogenes of the present invention; or their fragments, and therefore such an antibody may be used to diagnose the cancer by determining whether or not the proteins are expressed in the body fluid samples of the subject using the method known in the art, for example an enzyme linked immunosorbent assay (ELISA), a radioimmunoassay (RIA), a sandwich assay, western blotting or immunoblotting on the polyacrylamide gel, etc.

Also, the protooncogene of the present invention may be used to establish cancer cell lines that can continue to grow in an uncontrolled manner, and such a cell line may be, for example, produced from the tumorous tissue developed in the back of a nude mouse using fibroblast cell transfected with the protooncogenes. Such a cancer cell line may be effectively used for searching anticancer agents, etc.

Hereinafter, preferred examples of the present invention will be described in detail referring to the accompanying drawings, not is intended to limit the scope of the invention.

### EXAMPLE 1

### Cultivation of Tumor Cell and Separation of Total RNA

1-1: PIG5 PIG11 PIG16 PIG17, PIG19, PIG20, PIG21

(Step 1) Cultivation of Tumor Cell

In order to conduct the mRNA differential display method, a normal lung tissue was obtained, and a primary lung cancer tissue and a cancer tissue metastasized to the right lung were obtained from a lung cancer patient who has not been previously subject to the anticancer and/or radiation therapies upon surgery operation. A549 (American Type Culture Collection; ATCC Number CCL-185) was used as the human lung cancer cell line in the differential display method.

Cells obtained from the obtained tissues and the A549 lung cancer cell line were grown in a Waymouth's MB 752/1 medium (Gibco) containing 2 mM glutamine, 100 IU/ml penicillin, 100 μg/ml streptomycin and 10% fetal bovine serum (Gibco, U.S.). The culture cells used in this experiment are cells at the exponentially growing stage, and the cells showing a viability of at least 95% by a trypan blue dye exclusion test were used herein (see, Freshney, “Culture of Animal Cells: A Manual of Basic Technique” 2nd Ed., A. R. Liss, New York, 1987).

(Step 2) Separation of RNA and mRNA Differential Display Method

The total RNA samples were separated from the normal lung tissue, the primary lung cancer tissue, the metastatic lung cancer tissue and the A549 cell, each obtained in Step 1, using the commercially available system RNeasy total RNA kit (Qiagen Inc., Germany), and then DNA contaminants were removed from the RNA samples using the message clean kit (GenHunter Corp., Brookline, Mass., U.S.).

1-2: PIG6, PIG7, TRG2

(Step 1) Cultivation of Tumor Cell

In order to conduct the mRNA differential display method, a normal exocervical tissue was obtained from a patient suffering from an uterine myoma who has been subject to hysterectomy, and a primary cervical tumor tissue and a metastatic lymph node tumor tissue were obtained from an uterine cancer patient the who has not been previously subject to the anticancer and/or radiation therapies upon surgery operation. CUMC-6 (Kim, J. W. et al., Gynecol. Oncol. 62: 230-240, 1996) was used as the human cervical cancer cell line in the differential display method.

Cells obtained from the obtained tissues and the CUMC-6 cell line were grown in a Waymouth's MB 752/1 medium (Gibco) containing 2 mM glutamine, 100 IU/ml penicillin, 100 μg/ml streptomycin and 10% fetal bovine serum (Gibco, U.S.). The culture cells used in this experiment are cells at the exponentially growing stage, and the cells showing a viability of at least 95% by a trypan blue dye exclusion test were used herein (Freshney, “Culture of Animal Cells: A Manual of Basic Technique” 2nd Ed., A. R. Liss, New York, 1987).

(Step 2) Separation of RNA and mRNA Differential Display Method

The total RNA samples were separated from the normal exocervical tissue, the primary cervical tumor tissue, the metastatic lymph node tumor tissue and the CUMC-6 cell, each obtained in Step 1, using the commercially available system RNeasy total RNA kit (Qiagen Inc., Germany), and then DNA contaminants were removed from the RNA samples using the message clean kit (GenHunter Corp., Brookline, Mass., U.S.).

1-3: HCCRBP2 Cloning by Yeast Two-Hybrid Assay

A MATCHMAKER LexA Two-Hybrid System (Clontech. Laboratories) was used to search for a protein that binds to a protein product of the human protooncogene HCCR-1 gene (Genebank Accession No.: AF195651), and this experiment was conducted using the conventional reported method (Golemis, E. A., et al., Current Protocols in Molecular Biology John Wiley & Sons, Inc. Chapters 20.0 and 20.1, 1996).

Strains and vectors used in the following experiment are included in a commercially available Catalog #K1609-1 kit from the company Clontech.

A p8op-lacZ vector was transformed into a yeast strain EGY48, and then the transformed EGY48 strain was plated on an uracil-free synthetic dropout medium in a SD/-uracil/glucose plate to select colonies of the cell that grow therein. The strains selected thus was incubated in an SD/-uracil/glucose medium, and transformed using as a bait a vector obtained by inserting a HCCR-1 gene between the restriction ezymes BamHI and SalI of a vector pLexA. In order to confirm that the HCCR-1 gene cloned into the pLexA vector was expressed normally, a western blotting was conducted using a LexA antibody. As a result, a band was detected in a desired size of 6,465 kDa. The resultant colony was incubated in an SD/-uracil,-histidine/glucose medium, and then transformed by a human fetal brain-derived AD fusion library pBD42AD vector again. In order to confirm that the library binds to the bait, a colony lifting assay was carried out (Breeden, L. & Nasmyth, K., Cold Spring Harbor Symposium Quant. Bio. 50:643-650, 1985). If the library binds to the bait, blue colonies are formed in the plate containing X-gal. The yeast was incubated, and its DNA was extracted using a glass bead and transformed into E. coli KC8 using an electroporation method. The transformed E. coli KC8 was plated on a n M9 minimal medium to select transformants. Plasmid DNA was extracted from the resultant transformants, and then transformed into E. coli DH5 α again. DNA was extracted from the transformed E. coli and treated with HindIII to obtain a clone having a desired size of approximately 0.6 kb.

### EXAMPLE 2

### Differential Display Reverse Transcription-Polymerase Chain Reaction (DDRT-PCR)

The differential display reverse transcription was carried out using a slightly modified reverse transcription-polymerase chain reaction (RT-PCR) proposed by Liang, P. and A. B. Pardee.

2-1: PIG5

At first, reverse transcription was conducted on 0.2 μg of each of the total RNAs obtained in Step 1 of Example 1 using an anchored primer H-T11G (5′-AAGCTTTTTTTTTTTG-3′, RNAimage kit, Genhunter, Cor., MA, U.S.) set forth in SEQ ID NO: 3 as the anchored oligo-dT primer.

Then, a PCR reaction was carried out in the presence of 0.5 mM [α-³⁵S] dATP (1200 Ci/mmole) using the same anchored primer and the primer H-AP9 (SEQ ID NO: 4) (5′-AAGCTTTTGATCC-3′) among the random 5′-11-mer primers (RNAimage primer sets 1-5) H-AP 1 to 40. The PCR reaction was conducted under the following conditions: the total 40 amplification cycles consisting of a denaturation step at 95° C. for 40 seconds, an annealing step at 40° C. for 2 minutes and an extension step at 72° C. for 40 seconds, and followed by one final extension step at 72° C. for 5 minutes.

The PCR-amplified fragments were dissolved in a 6% polyacrylamide sequencing gel for DNA sequence, and then a position of a differentially expressed band was confirmed using autoradiography.

A 192-base pair (bp) band with L699 cDNA (Base positions from 778 to 969 of SEQ ID NO: 1) was extracted from the dried gel. The extracted gel was heated for 15 minutes to elute the L699 cDNA, and then the PCR reaction was repeated with the same primer under the same condition as described above to re-amplify the L699 cDNA, except that [α-³⁵S]-labeled dATP (1200 Ci/mmole) and 20 μM dNTP were not used herein.

2-2: PIG6

At first, reverse transcription was conducted on 0.2 μg of each of the total RNAs obtained in Step 1 of Example 1 using an anchored primer H-T11A (5′-AAGCTTTTTTTTTTTA-3′, RNAimage kit, Genhunter, Cor., MA, U.S.) having a DNA sequence set forth in SEQ ID NO: 7 as the anchored oligo-dT primer.

Then, a PCR reaction was carried out in the presence of 0.5 mM [α-³⁵S] dATP (1200 Ci/mmole) using the same anchored primer and the primer H-AP32 (5′-AAGCTTCCTGCAA-3′) having a DNA sequence set forth in SEQ ID NO: 8 among the random 5′-11-mer primers (RNAimage primer sets 1-5) H-AP 1 to 40. The PCR reaction was conducted under the following conditions: the total 40 amplification cycles consisting of a denaturation step at 95° C. for 40 seconds, an annealing step at 40° C. for 2 minutes and an extension step at 72° C. for 40 seconds, and followed by one final extension step at 72° C. for 5 minutes.

The PCR-amplified fragments were dissolved in a 6% polyacrylamide sequencing gel for DNA sequence, and then a position of a differentially expressed band was confirmed using autoradiography.

A 392-base pair (bp) band with CA325 cDNA (Base positions from 2485 to 2876 of SEQ ID NO: 5) was extracted from the dried gel. The extracted gel was heated for 15 minutes to elute the CA325 cDNA, and then the PCR reaction was repeated with the same primer under the same condition as described above to re-amplify the CA325 cDNA, except that [α-³⁵S]-labeled dATP (1200 Ci/mmole) and 20 μM dNTP were not used herein.

2-3: PIG7

At first, reverse transcription was conducted on 0.2 μg of each of the total RNAs obtained in Step 1 of Example 1 using an anchored primer H-T11A (5′-AAGCTTTTTTTTTTTA-3′, RNAimage kit, Genhunter, Cor., MA, U.S.) having a DNA sequence set forth in SEQ ID NO: 11 as the anchored oligo-dT primer.

Then, a PCR reaction was carried out in the presence of 0.5 mM [α-³⁵S] dATP (1200 Ci/mmole) using the same anchored primer and the primer H-AP27 (5′-AAGCTTCTGCTGG-3′) having a DNA sequence set forth in SEQ ID NO: 12 among the random 5′-11-mer primers (RNAimage primer sets 1-5) H-AP 1 to 40. The PCR reaction was conducted under the following conditions: the total 40 amplification cycles consisting of a denaturation step at 95° C. for 40 seconds, an annealing step at 40° C. for 2 minutes and an extension step at 72° C. for 40 seconds, and followed by one final extension step at 72° C. for 5 minutes.

The PCR-amplified fragments were dissolved in a 6% polyacrylamide sequencing gel for DNA sequence, and then a position of a differentially expressed band was confirmed using autoradiography.

A 368-base pair (bp) band with CA273 cDNA (Base positions from 3762 to 4129 of SEQ ID NO: 9) was extracted from the dried gel. The extracted gel was heated for 15 minutes to elute the CA273 cDNA, and then the PCR reaction was repeated with the same primer under the same condition as described above to re-amplify the CA273 cDNA, except that [α-³⁵S]-labeled dATP (1200 Ci/mmole) and 20 μM dNTP were not used herein.

2-4: PIG11

At first, reverse transcription was conducted on 0.2 μg of each of the total RNAs obtained in Step 1 of Example 1 using an anchored primer H-T11G (5′-AAGCTTTTTTTTTTTA-3′, RNAimage kit, Genhunter, Cor., MA, U.S.) having a DNA sequence set forth in SEQ ID NO: 15 as the anchored oligo-dT primer.

Then, a PCR reaction was carried out in the presence of 0.5 mM [α-³⁵S] dATP (1200 Ci/mmole) using the same anchored primer and the primer H-AP 11 (SEQ ID NO: 16) (5′-AAGCTTCGGGTAA-3′) among the random 5′-11-mer primers (RNAimage primer sets 1-5) H-AP 1 to 40. The PCR reaction was conducted under the following conditions: the total 40 amplification cycles consisting of a denaturation step at 95° C. for 40 seconds, an annealing step at 40° C. for 2 minutes and an extension step at 72° C. for 40 seconds, and followed by one final extension step at 72° C. for 5 minutes.

The PCR-amplified fragments were dissolved in a 6% polyacrylamide sequencing gel for DNA sequence, and then a position of a differentially expressed band was confirmed using autoradiography.

A 203-base pair (bp) band with L667 cDNA (Base positions from 796 to 998 of SEQ ID NO: 13) was extracted from the dried gel. The extracted gel was heated for 15 minutes to elute the L667 cDNA, and then the PCR reaction was repeated with the same primer under the same condition as described above to re-amplify the L667 cDNA, except that [α-³⁵S]-labeled dATP (1200 Ci/mmole) and 20 μM dNTP were not used herein.

2-5: PIG16

At first, reverse transcription was conducted on 0.2 μg of each of the total RNAs obtained in Step 1 of Example 1 using an anchored primer H-T11C (5′-AAGCTTTTTTTTTTTC-3′, RNAimage kit, Genhunter, Cor., MA, U.S.) having a DNA sequence set forth in SEQ ID NO: 19 as the anchored oligo-dT primer.

Then, a PCR reaction was carried out in the presence of 0.5 mM [α-3S] dATP (1200 Ci/mmole) using the same anchored primer and the primer H-AP16 (SEQ ID NO: 20) (5′-AAGCTTTAGAGCG-3′) among the random 5′-11-mer primers (RNAimage primer sets 1-5) H-AP 1 to 40. The PCR reaction was conducted under the following conditions: the total 40 amplification cycles consisting of a denaturation step at 95° C. for 40 seconds, an annealing step at 40° C. for 2 minutes and an extension step at 72° C. for 40 seconds, and followed by one final extension step at 72° C. for 5 minutes.

The PCR-amplified fragments were dissolved in a 6% polyacrylamide sequencing gel for DNA sequence, and then a position of a differentially expressed band was confirmed using autoradiography.

A 322-base pair (bp) band with L668 cDNA (Base positions from 1277 to 1598 of SEQ ID NO: 17) was extracted from the dried gel. The extracted gel was heated for 15 minutes to elute the L668 cDNA, and then the PCR reaction was repeated with the same primer under the same condition as described above to re-amplify the L668 cDNA, except that [α-³⁵S]-labeled dATP (1200 Ci/mmole) and 20 μM dNTP were not used herein.

2-6: PIG17

At first, reverse transcription was conducted on 0.2 μg of each of the total RNAs obtained in Step 1 of Example 1 using an anchored primer H-T11C (5′-AAGCTTTTTTTTTTTC-3′, RNAimage kit, Genhunter, Cor., MA, U.S.) having a DNA sequence set forth in SEQ ID NO: 23 as the anchored oligo-dT primer.

Then, a PCR reaction was carried out in the presence of 0.5 mM [α-³⁵S] dATP (1200 Ci/mmole) using the same anchored primer and the primer H-AP18 (SEQ ID NO: 24) (5′-AAGCTTAGAGGCA-3′) among the random 5′-11-mer primers (RNAimage primer sets 1-5) H-AP 1 to 40. The PCR reaction was conducted under the following conditions: the total 40 amplification cycles consisting of a denaturation step at 95° C. for 40 seconds, an annealing step at 40° C. for 2 minutes and an extension step at 72° C. for 40 seconds, and followed by one final extension step at 72° C. for 5 minutes.

The PCR-amplified fragments were dissolved in a 6% polyacrylamide sequencing gel for DNA sequence, and then a position of a differentially expressed band was confirmed using autoradiography.

A 211-base pair (bp) band with L211 cDNA (Base positions from 389 to 599 of SEQ ID NO: 21) was extracted from the dried gel. The extracted gel was heated for 15 minutes to elute the L211 cDNA, and then the PCR reaction was repeated with the same primer under the same condition as described above to re-amplify the L211 cDNA, except that [α-³⁵S]-labeled dATP (1200 Ci/mmole) and 20 μM dNTP were not used herein.

2-7: PIG19

At first, reverse transcription was conducted on 0.2 μg of each of the total RNAs obtained in Step 1 of Example 1 using an anchored primer H-T11G (5′-AAGCTTTTTTTTTTTG-3′, RNAimage kit, Genhunter, Cor., MA, U.S.) having a DNA sequence set forth in SEQ ID NO: 27 as the anchored oligo-dT primer.

Then, a PCR reaction was carried out in the presence of 0.5 mM [α-³⁵S] dATP (1200 Ci/mmole) using the same anchored primer and the primer H-AP16 (SEQ ID NO: 28) (5′-AAGCTTTAGAGCG-3′) among the random 5′-11-mer primers (RNAimage primer sets 1-5) H-AP 1 to 40. The PCR reaction was conducted under the following conditions: the total 40 amplification cycles consisting of a denaturation step at 95° C. for 40 seconds, an annealing step at 40° C. for 2 minutes and an extension step at 72° C. for 40 seconds, and followed by one final extension step at 72° C. for 5 minutes.

The PCR-amplified fragments were dissolved in a 6% polyacrylamide sequencing gel for DNA sequence, and then a position of a differentially expressed band was confirmed using autoradiography.

A 233-base pair (bp) band with L722 cDNA (Base positions from 777 to 1009 of SEQ ID NO: 25) was extracted from the dried gel. The extracted gel was heated for 15 minutes to elute the L722 cDNA, and then the PCR reaction was repeated with the same primer under the same condition as described above to re-amplify the L722 cDNA, except that [α-³⁵S]-labeled dATP (1200 Ci/mmole) and 20 μM dNTP were not used herein.

2-8: PIG20

At first, reverse transcription was conducted on 0.2 μg of each of the total RNAs obtained in Step 1 of Example 1 using an anchored primer H-T11G (5′-AAGCTTTTTTTTTTTG-3′, RNAimage kit, Genhunter, Cor., MA, U.S.) having a DNA sequence set forth in SEQ ID NO: 31 as the anchored oligo-dT primer.

Then, a PCR reaction was carried out in the presence of 0.5 mM [α-³⁵S] dATP (1200 Ci/mmole) using the same anchored primer and the primer H-AP17 (SEQ ID NO: 32) (5′-AAGCTTACCAGGT-3′) among the random 5′-11-mer primers (RNAimage primer sets 1-5) H-AP 1 to 40. The PCR reaction was conducted under the following conditions: the total 40 amplification cycles consisting of a denaturation step at 95° C. for 40 seconds, an annealing step at 40° C. for 2 minutes and an extension step at 72° C. for 40 seconds, and followed by one final extension step at 72° C. for 5 minutes.

The PCR-amplified fragments were dissolved in a 6% polyacrylamide sequencing gel for DNA sequence, and then a position of a differentially expressed band was confirmed using autoradiography.

A 211-base pair (bp) band with L752 cDNA (Base positions from 304 to 514 of SEQ ID NO: 29) was extracted from the dried gel. The extracted gel was heated for 15 minutes to elute the L752 cDNA, and then the PCR reaction was repeated with the same primer under the same condition as described above to re-amplify the L752 cDNA, except that [α-³⁵S]-labeled dATP (1200 Ci/mmole) and 20 μM dNTP were not used herein.

2-9: PIG21

At first, reverse transcription was conducted on 0.2 μg of each of the total RNAs obtained in Step 1 of Example 1 using an anchored primer H-T11C (5′-AAGCTTTTTTTTTTTC-3′, RNAimage kit, Genhunter, Cor., MA, U.S.) having a DNA sequence set forth in SEQ ID NO: 35 as the anchored oligo-dT primer.

Then, a PCR reaction was carried out in the presence of 0.5 mM [α-³⁵S] dATP (1200 Ci/mmole) using the same anchored primer and the primer H-AP15 (SEQ ID NO: 36) (5′-AAGCTTACGCAAC-3′) among the random 5′-11-mer primers (RNAimage primer sets 1-5) H-AP 1 to 40. The PCR reaction was conducted under the following conditions: the total 40 amplification cycles consisting of a denaturation step at 95° C. for 40 seconds, an annealing step at 40° C. for 2 minutes and an extension step at 72° C. for 40 seconds, and followed by one final extension step at 72° C. for 5 minutes.

The PCR-amplified fragments were dissolved in a 6% polyacrylamide sequencing gel for DNA sequence, and then a position of a differentially expressed band was confirmed using autoradiography.

A 272-base pair (bp) band with L1003 cDNA (Base positions from 665 to 936 of SEQ ID NO: 33) was extracted from the dried gel. The extracted gel was heated for 15 minutes to elute the L1003 cDNA, and then the PCR reaction was repeated with the same primer under the same condition as described above to re-amplify the L1003 cDNA, except that [α-³⁵S]-labeled dATP (1200 Ci/mmole) and 20 μM dNTP were not used herein.

2-10: TRG2

At first, reverse transcription was conducted on 0.2 μg of each of the total RNAs obtained in Step 1 of Example 1 using an anchored primer H-T11A (5′-AAGCTTTTTTTTTTTA-3′, RNAimage kit, Genhunter, Cor., MA, U.S.) having a DNA sequence set forth in SEQ ID NO: 41 as the anchored oligo-dT primer.

Then, a PCR reaction was carried out in the presence of 0.5 mM [α-³⁵S] dATP (1200 Ci/mmole) using the same anchored primer and the primer H-AP32 (5′-AAGCTTCCTGCAA-3′) having a DNA sequence set forth in SEQ ID NO: 42 among the random 5′-11-mer primers (RNAimage primer sets 1-5) H-AP 1 to 40. The PCR reaction was conducted under the following conditions: the total 40 amplification cycles consisting of a denaturation step at 95° C. for 40 seconds, an annealing step at 40° C. for 2 minutes and an extension step at 72° C. for 40 seconds, and followed by one final extension step at 72° C. for 5 minutes.

The PCR-amplified fragments were dissolved in a 6% polyacrylamide sequencing gel for DNA sequence, and then a position of a differentially expressed band was confirmed using autoradiography.

A 373-base pair (bp) band with HP90-811 cDNA (Base positions from 1797 to 2169 of SEQ ID NO: 39) was extracted from the dried gel. The extracted gel was heated for 15 minutes to elute the HP90-811 cDNA, and then the PCR reaction was repeated with the same primer under the same condition as described above to re-amplify the HP90-811 cDNA, except that [α-³⁵S]-labeled dATP (1200 Ci/mmole) and 20 μM dNTP were not used herein.

### EXAMPLE 3

### Cloning

PIG5, PIG6, PIG7, PIG11, PIG16, PIG17, PIG19, PIG20, PIG21, TRG2

The L699 PCR product; the CA325 PCR product; the CA273 PCR product; the L667 PCR product; the L668 PCR product; the L211 PCR product; the L722 PCR product; the L752 PCR product; the L1003 PCR product; and the HP90-811 PCR product, which were all re-amplified as described above, were inserted into a pGEM-T EASY vector, respectively, according to the manufacturer's manual using the TA cloning system (Promega, U.S.).

(Step 1) Ligation Reaction

2 μl of each of the L699 PCR product; the CA325 PCR product; the CA273 PCR product; the L667 PCR product; the L668 PCR product; the L211 PCR product; the L722 PCR product; the L752 PCR product; the L1003 PCR product and the HP90-811 PCR product, which were all re-amplified in Example 2, 1 μl of pGEM-T EASY vector (50 ng), 1 μl of T4 DNA ligase (10× buffer) and 1 μl of T4 DNA ligase (3 weiss units/μl; Promega) were put into a 0.5 mΩ test tube, and distilled water was added thereto to a final volume of 10 μl. The ligation reaction mixtures were incubated overnight at 14° C.

(Step 2) Transformation of TA Clone

E. coli JM109 (Promega, Wis., U.S.) was incubated in 10 ml of LB broth (10 g of bacto-tryptone, 5 g of bacto-yeast extract, 5 g of NaCl) until the optical density at 600 nm reached approximately 0.3 to 0.6. The incubated mixture was kept in ice at about 10 minutes and centrifuged at 4,000 rpm for 10 minutes at 4° C., and then the supernatant wad discarded and the cell was collected. The collected cell pellet was exposed to 10 ml of 0.1 M ice-cold CaCl₂ for approximately 30 minutes to 1 hours to produce a competent cell. The product was centrifuged again at 4,000 rpm for 10 minutes at 4° C., and then the supernatant wad discarded and the cell was collected and suspended in 2 ml of 0.1 M ice-cold CaCl₂.

200 μl of the competent cell suspension was transferred to a new microfuge, and 2 μl of the ligation reaction product prepared in Step 1 was added thereto. The resultant mixture was incubated in a water bath at 42° C. for 90 seconds, and then quenched at 0° C. 800 μl of SOC medium (2.0 g of bacto-tryptone, 0.5 g of bacto-yeast extract, 1 ml of 1 M NaCl, 0.25 ml of 1 M KCl, 97 ml of TDW, 1 ml of 2 M Mg²⁺, 1 ml of 2 M glucose) was added thereto and the resultant mixture was incubated at 37° C. for 45 minutes in a rotary shaking incubator at 220 rpm.

25 μl of X-gal (stored in 40 mg/ml of dimethylformamide) was spread with a glass rod on a LB plate supplemented with ampicillin and previously put into the incubator at 37° C., and 25 μl of transformed cell was added thereto and spread again with a glass rod, and then incubated overnight at 37° C. After incubation, the 3 to 4 formed white colonies was selected to seed-culture each of the selected cells in a LB plate supplemented with ampicillin. In order to construct a plasmid, the colonies considered to be colonies into which the ligation reaction products were introduced respectively, namely the transformed E. coli strains JM109/L699; JM109/CA325; JM109/CA273; JM109/L667; JM109/L668; JM109/L211; JM109/L699; JM109/L752; JM109/L1003; and JM109/HP90-811 were selected and incubated in 10 ml of terrific broth (900 ml of TDW, 12 g of bacto-tryptone, 24 g of bacto-yeast extract, 4 ml of glycerol, 0.17 M KH₂PO₄, 100 ml of 0.72 N K₂HPO₄).

### EXAMPLE 4

### Separation of Recombinant Plasmid DNA

PIG5, PIG6, PIG7, PIG11, PIG16, PIG17, PIG19, PIG20, PIG21, TRG2

Each of the L699 plasmid DNA; the CA325 plasmid DNA; the CA273 plasmid DNA; the L667 plasmid DNA; the L668 plasmid DNA; the L211 plasmid DNA; the L722 plasmid DNA; the L752 plasmid DNA; the L1003 plasmid DNA and the HP90-8115 plasmid DNA was separated from the transformed E. coli strains according to the manufacturer's manual using a Wizard™ Plus Minipreps DNA purification kit (Promega, U.S.).

It was confirmed that some of each of the separated plasmid DNAs was treated with a restriction enzyme ECoRI, and partial sequences of L699; CA325; CA273; L667; L668; L211; L722; L752; L1003; and HP90-8115 was inserted into the plasmid, respectively, by conducting electrophoresis in a 2% gel.

### EXAMPLE 5

### DNA Sequencing Analysis

5-1: PIG5

The L699 PCR product obtained in Example 2 was PCR-amplified, cloned, and then re-amplified according to the conventional method. The resultant L699 PCR fragment was sequenced according to a dideoxy chain termination method using the Sequenase version 2.0 DNA sequencing kit (United States Biochemical, Cleveland, Ohio, U.S.).

The DNA sequence of the said gene corresponds to nucleotide sequence positions from 778 to 969 of SEQ ID NO: 1, which is named “L699” in the present invention.

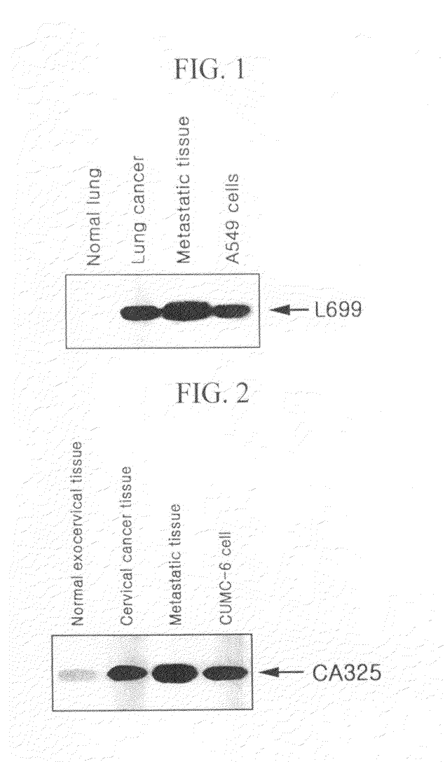

The 192-bp cDNA fragment obtained above, for example L699 was subject to the differential display reverse transcription-polymerase chain reaction (DDRT-PCR) using a 5′-random primer H-AP9 and a 3′-anchored primer H-T11G, and then confirmed using the electrophoresis. As shown in FIG. 1, it was revealed from the differential display (DD) that the gene was differentially expressed in the normal lung tissue, the left lung cancer tissue, the metastatic lung cancer tissue metastasized from the left lung to the right lung, and the A549 lung cancer cell. As shown in FIG. 1, the 192-bp cDNA fragment L699 was expressed in the lung cancer tissue, the metastatic lung cancer tissue and the A549 lung cancer cell, but not expressed in the normal lung tissue.

5-2: PIG6

The CA325 PCR product obtained in Example 2 was amplified, cloned, and then re-amplified according to the conventional method. The resultant CA325 PCR fragment was sequenced according to a dideoxy chain termination method using the Sequenase version 2.0 DNA sequencing kit (United States Biochemical, Cleveland, Ohio, U.S.).

The DNA sequence of the said gene corresponds to nucleotide sequence positions from 2485 to 2876 of SEQ ID NO: 5, which is named “CA325” in the present invention.

The 392-bp cDNA fragment obtained above, for example CA325 was subject to the differential display reverse transcription-polymerase chain reaction (DDRT-PCR) using a 5′-random primer H-AP32 and a 3′-anchored primer H-T11A, and then confirmed using the electrophoresis.

As shown in FIG. 2, it was revealed from the differential display (DD) that the gene was differentially expressed in the normal exocervical tissue, the metastatic lymph node tissue and the CUMC-6 cell. As shown in FIG. 2, the 392-bp cDNA fragment CA325 was expressed in the cervical cancer, the metastatic lymph node tissue and the CUMC-6 cancer cell, but very slightly expressed in the normal tissue.

5-3: PIG7

The CA273 PCR product obtained in Example 2 was amplified, cloned, and then re-amplified according to the conventional method. The resultant CA273 PCR fragment was sequenced according to a dideoxy chain termination method using the Sequenase version 2.0 DNA sequencing kit (United States Biochemical, Cleveland, Ohio, U.S.).

The DNA sequence of the said gene corresponds to nucleotide sequence positions from 3762 to 4129 of SEQ ID NO: 9, which is named “CA273” in the present invention.

The 368-bp cDNA fragment obtained above, for example CA273 was subject to the differential display reverse transcription-polymerase chain reaction (DDRT-PCR) using a 5′-random primer H-AP27 and a 3′-anchored primer H-T11A, and then confirmed using the electrophoresis.

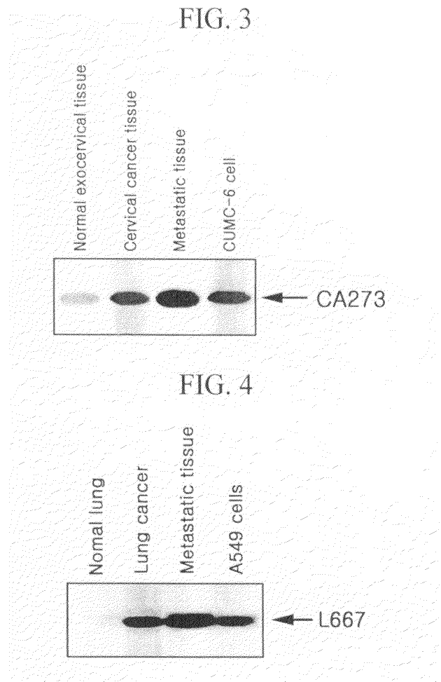

As shown in FIG. 3, it was revealed from the differential display (DD) that the gene was differentially expressed in the normal exocervical tissue, the metastatic lymph node tissue and the CUMC-6 cell. As shown in FIG. 3, the 368-bp cDNA fragment CA273 was expressed in the cervical cancer, the metastatic lymph node tissue and the CUMC-6 cancer cell, but very slightly expressed in the normal tissue.

5-4: PIG11

The L667 PCR product obtained in Example 2 was PCR-amplified, cloned, and then re-amplified according to the conventional method. The resultant L667 PCR fragment was sequenced according to a dideoxy chain termination method using the Sequenase version 2.0 DNA sequencing kit (United States Biochemical, Cleveland, Ohio, U.S.).

The DNA sequence of the said gene corresponds to nucleotide sequence positions from 796 to 998 of SEQ ID NO: 13, which is named “L667” in the present invention.

The 203-bp cDNA fragment obtained above, for example L667 was subject to the differential display reverse transcription-polymerase chain reaction (DDRT-PCR) using a 5′-random primer H-AP11 and a 3′-anchored primer H-T11A, and then confirmed using the electrophoresis. As shown in FIG. 4, it was revealed from the differential display (DD) that the gene was differentially expressed in the normal lung tissue, the left lung cancer tissue, the metastatic lung cancer tissue metastasized from the left lung to the right lung, and the A549 lung cancer cell. As shown in FIG. 4, the 203-bp cDNA fragment L667 was expressed in the lung cancer tissue, the metastatic lung cancer tissue and the A549 lung cancer cell, but not expressed in the normal lung tissue.

5-5: PIG16

The L668 PCR product obtained in Example 2 was PCR-amplified, cloned, and then re-amplified according to the conventional method. The resultant L668 PCR fragment was sequenced according to a dideoxy chain termination method using the Sequenase version 2.0 DNA sequencing kit (United States Biochemical, Cleveland, Ohio, U.S.).

The DNA sequence of the said gene corresponds to nucleotide sequence positions from 1277 to 1598 of SEQ ID NO: 17, which is named “L668” in the present invention.

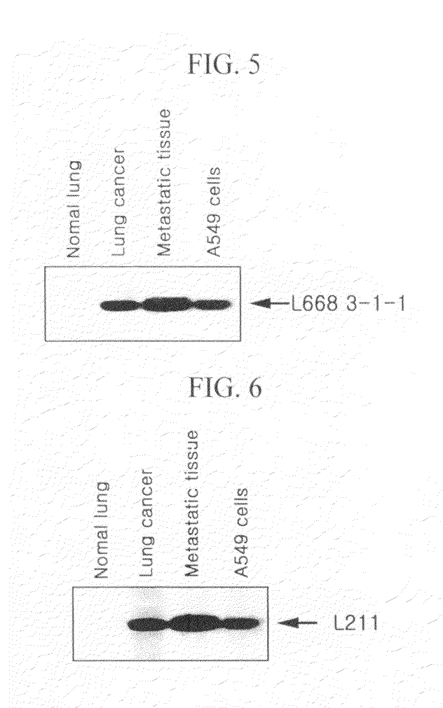

The 322-bp cDNA fragment obtained above, for example L668 was subject to the differential display reverse transcription-polymerase chain reaction (DDRT-PCR) using a 5′-random primer H-AP16 and a 3′-anchored primer H-TI IC, and then confirmed using the electrophoresis. As shown in FIG. 5, it was revealed from the differential display (DD) that the gene was differentially expressed in the normal lung tissue, the left lung cancer tissue, the metastatic lung cancer tissue metastasized from the left lung to the right lung, and the A549 lung cancer cell. As shown in FIG. 5, the 322-bp cDNA fragment L668 was expressed in the lung cancer tissue, the metastatic lung cancer tissue and the A549 lung cancer cell, but not expressed in the normal lung tissue.

5-6: PIG17

The L211 PCR product obtained in Example 2 was PCR-amplified, cloned, and then re-amplified according to the conventional method. The resultant L211 PCR fragment was sequenced according to a dideoxy chain termination method using the Sequenase version 2.0 DNA sequencing kit (United States Biochemical, Cleveland, Ohio, U.S.).

The DNA sequence of the said gene corresponds to nucleotide sequence positions from 389 to 599 of SEQ ID NO: 21, which is named “L211” in the present invention.

The 211-bp cDNA fragment obtained above, for example L211 was subject to the differential display reverse transcription-polymerase chain reaction (DDRT-PCR) using a 5′-random primer H-AP18 and a 3′-anchored primer H-T11C, and then confirmed using the electrophoresis. As shown in FIG. 6, it was revealed from the differential display (DD) that the gene was differentially expressed in the normal lung tissue, the left lung cancer tissue, the metastatic lung cancer tissue metastasized from the left lung to the right lung, and the A549 lung cancer cell. As shown in FIG. 6, the 211-bp cDNA fragment L211 was expressed in the lung cancer tissue, the metastatic lung cancer tissue and the A549 lung cancer cell, but not expressed in the normal lung tissue.

5-7: PIG19

The L722 PCR product obtained in Example 2 was PCR-amplified, cloned, and then re-amplified according to the conventional method. The resultant L722 PCR fragment was sequenced according to a dideoxy chain termination method using the Sequenase version 2.0 DNA sequencing kit (United States Biochemical, Cleveland, Ohio, U.S.).

The DNA sequence of the said gene corresponds to nucleotide sequence positions from 777 to 1009 of SEQ ID NO: 25, which is named “L722” in the present invention.

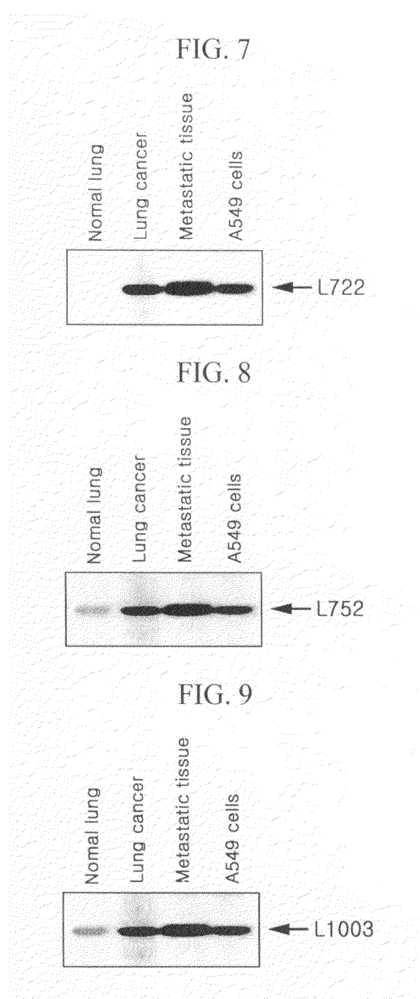

The 233-bp cDNA fragment obtained above, for example L722 was subject to the differential display reverse transcription-polymerase chain reaction (DDRT-PCR) using a 5′-random primer H-AP16 and a 3′-anchored primer H-T11G, and then confirmed using the electrophoresis. As shown in FIG. 7, it was revealed from the differential display (DD) that the gene was differentially expressed in the normal lung tissue, the left lung cancer tissue, the metastatic lung cancer tissue metastasized from the left lung to the right lung, and the A549 lung cancer cell. As shown in FIG. 7, the 233-bp cDNA fragment L722 was expressed in the lung cancer tissue, the metastatic lung cancer tissue and the A549 lung cancer cell, but not expressed in the normal lung tissue.

5-8: PIG20

The L752 PCR product obtained in Example 2 was PCR-amplified, cloned, and then re-amplified according to the conventional method. The resultant L752 PCR fragment was sequenced according to a dideoxy chain termination method using the Sequenase version 2.0 DNA sequencing kit (United States Biochemical, Cleveland, Ohio, U.S.).

The DNA sequence of the said gene corresponds to nucleotide sequence positions from 304 to 514 of SEQ ID NO: 29, which is named “L752” in the present invention.

The 211-bp cDNA fragment obtained above, for example L752 was subject to the differential display reverse transcription-polymerase chain reaction (DDRT-PCR) using a 5′-random primer H-AP17 and a 3′-anchored primer H-T11G, and then confirmed using the electrophoresis. As shown in FIG. 8, it was revealed from the differential display (DD) that the gene was differentially expressed in the normal lung tissue, the left lung cancer tissue, the metastatic lung cancer tissue metastasized from the left lung to the right lung, and the A549 lung cancer cell. As shown in FIG. 8, The 211-bp cDNA fragment L752 was expressed in the lung cancer tissue, the metastatic lung cancer tissue and the A549 lung cancer cell, but very slightly expressed in the normal lung tissue.

5-9: PIG21

The L1003 PCR product obtained in Example 2 was PCR-amplified, cloned, and then re-amplified according to the conventional method. The resultant L1003 PCR fragment was sequenced according to a dideoxy chain termination method using the Sequenase version 2.0 DNA sequencing kit (United States Biochemical, Cleveland, Ohio, U.S.).

The DNA sequence of the said gene corresponds to nucleotide sequence positions from 665 to 936 of SEQ ID NO: 33, which is named “L1003” in the present invention.

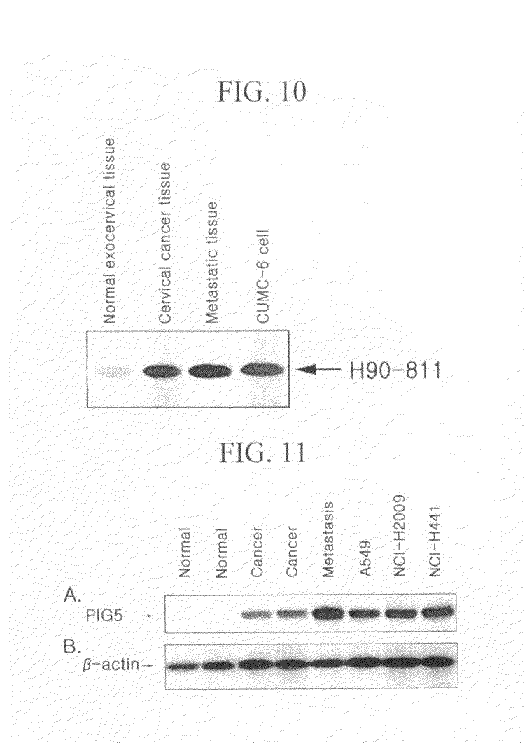

The 272-bp cDNA fragment obtained above, for example L1003 was subject to the differential display reverse transcription-polymerase chain reaction (DDRT-PCR) using a 5′-random primer H-AP15 and a 3′-anchored primer H-T11C, and then confirmed using the electrophoresis. As shown in FIG. 9, it was revealed from the differential display (DD) that the gene was differentially expressed in the normal lung tissue, the left lung cancer tissue, the metastatic lung cancer tissue metastasized from the left lung to the right lung, and the A549 lung cancer cell. As shown in FIG. 9, The 272-bp cDNA fragment L1003 was expressed in the lung cancer tissue, the metastatic lung cancer tissue and the A549 lung cancer cell, but very slightly expressed in the normal lung tissue.

5-10: TRG2

The HP90-811 PCR product obtained in Example 2 was PCR-amplified, cloned, and then re-amplified according to the conventional method. The resultant HP90-811 PCR fragment was sequenced according to a dideoxy chain termination method using the Sequenase version 2.0 DNA sequencing kit (United States Biochemical, Cleveland, Ohio, U.S.).

The DNA sequence of the said gene corresponds to nucleotide sequence positions from 1797 to 2169 of SEQ ID NO: 39, which is named “HP90-81” in the present invention.

The 373-bp cDNA fragment obtained above, for example HP90-811 was subject to the differential display reverse transcription-polymerase chain reaction (DDRT-PCR) using a 5′-random primer H-AP32 and a 3′-anchored primer H-T11A, and then confirmed using the electrophoresis.

As shown in FIG. 10, it was revealed from the differential display (DD) that the gene was differentially expressed in the normal exocervical tissue, the metastatic lymph node tissue and the CUMC-6 cell. As shown in FIG. 10, The 373-bp cDNA fragment HP90-811 was expressed in the cervical cancer, the metastatic lymph node tissue and the CUMC-6 cancer cell, but very slightly expressed in the normal tissue.

### EXAMPLE 6

### Full-length cDNA Sequence Analysis of Protooncogene

6-1: PIG5

The ³²P-labeled L699 was used as the probe to screen a bacteriophage λ gt11 human lung embryonic fibroblast cDNA library (Miki, T. et al., Gene 83: 137-146, 1989), thereby to obtain a full-length gene having the L699 cDNA sequence. Two full-length genes were obtained from the human lung embryonic fibroblast cDNA library; one gene is a full-length PIG5 cDNA clone in which the 1009-bp fragment was inserted into the pCEV-LAC vector, and then deposited with Accession No. AY236486 into the GenBank database of U.S. NIH on Feb. 13, 2003 (Publication Date: Dec. 31, 2004).

The PIG5 clone inserted into the λ pCEV vector was cleaved by the restriction enzyme NotI and isolated from the phage in the form of ampicillin-resistant pCEV-LAC phagemid vector (see the above reference).

The pCEV-LAC vector containing the PIG5 gene was ligated by T4 DNA ligase to obtain PIG5 plasmid DNA, and then E. coli DH5 α was transformed with the ligated clone.

A 1009-bp full-length sequence of the PIG5 was set forth in SEQ ID NO: 1.

In the DNA sequence of SEQ ID NO: 1, it is estimated that a full-length open reading frame of the protooncogene of the present invention corresponds to nucleotide sequence positions from 9 to 746, and encodes a protein consisting of 245 amino acids of SEQ ID NO: 2.

6-2: PIG6

The ³²P-labeled CA325 was used as the probe to screen a bacteriophage λ gt11 human lung embryonic fibroblast cDNA library (Miki, T. et al., Gene 83: 137-146, 1989). A full-length PIG6 cDNA clone, in which the 2964-bp fragment was inserted into the pCEV-LAC vector, was obtained from the human lung embryonic fibroblast cDNA library, and then deposited with Accession No. AY236487 into the GenBank database of U.S. NIH on Feb. 13, 2003 (Publication Date: Dec. 31, 2004).

The PIG6 clone inserted into the λ pCEV vector was cleaved by the restriction enzyme NotI and isolated from the phage in the form of ampicillin-resistant pCEV-LAC phagemid vector (Miki, T. et al., Gene 83: 137-146, 1989).

The pCEV-LAC vector containing the PIG6 gene was ligated by T4 DNA ligase to obtain PIG6 plasmid DNA, and then E. coli DH5 α was transformed with the ligated clone.

A 22964-bp full-length sequence of the PIG6 was set forth in SEQ ID NO: 5.

In the DNA sequence of SEQ ID NO: 1, it is estimated that a full-length open reading frame of the protooncogene of the present invention corresponds to nucleotide sequence positions from 293 to 2302, and encodes a protein consisting of 669 amino acids of SEQ ID NO: 6.

6-3: PIG7

The ³²P-labeled CA273 was used as the probe to screen a bacteriophage λ gt11 human lung embryonic fibroblast cDNA library (Miki, T. et al., Gene 83: 137-146, 1989). A full-length PIG7 cDNA clone, in which the 4301-bp fragment was inserted into the pCEV-LAC vector, was obtained from the human lung embryonic fibroblast cDNA library, and then deposited with Accession No. AY236488 into the GenBank database of U.S. NIH on Feb. 13, 2003 (Publication Date: Dec. 31, 2004).

The PIG7 clone inserted into the λ pCEV vector was cleaved by the restriction enzyme NotI and isolated from the phage in the form of ampicillin-resistant pCEV-LAC phagemid vector (Miki, T. et al., Gene 83: 137-146, 1989).

The pCEV-LAC vector containing the PIG7 gene was ligated by T4 DNA ligase to obtain PIG7 plasmid DNA, and then E. coli DH5 α was transformed with the ligated clone.

A 4301-bp full-length sequence of the PIG7 was set forth in SEQ ID NO: 9.

In the DNA sequence of SEQ ID NO: 9, it is estimated that a full-length open reading frame of the protooncogene of the present invention corresponds to nucleotide sequence positions from 3556 to 3792, and encodes a protein consisting of 78 amino acids of SEQ ID NO: 10.

6-4: PIG11

The ³²P-labeled L667 was used as the probe to screen a bacteriophage λ gt11 human lung embryonic fibroblast cDNA library (Miki, T. et al., Gene 83: 137-146, 1989), thereby to obtain a full-length gene having the L667 cDNA sequence. Two full-length genes were obtained from the human lung embryonic fibroblast cDNA library; one gene is a full-length PIG11 cDNA clone in which the 1038-bp fragment was inserted into the pCEV-LAC vector, and then deposited with Accession No. AY258284 into the GenBank database of U.S. NIH on Feb. 24, 2003 (Publication Date: Dec. 31, 2004).

The PIG11 clone inserted into the λ pCEV vector was cleaved by the restriction enzyme NotI and isolated from the phage in the form of ampicillin-resistant pCEV-LAC phagemid vector (see the above reference).

The pCEV-LAC vector containing the PIG11 gene was ligated by T4 DNA ligase to obtain PIG11 plasmid DNA, and then E. coli DH5 α was transformed with the ligated clone.

A 1038-bp full-length sequence of the PIG11 was set forth in SEQ ID NO: 13.

In the DNA sequence of SEQ ID NO: 13, it is estimated that a full-length open reading frame of the protooncogene of the present invention corresponds to nucleotide sequence positions from 50 to 931, and encodes a protein consisting of 293 amino acids of SEQ ID NO: 14.

6-5: PIG16

The ³²P-labeled L668 was used as the probe to screen a bacteriophage λ gt11 human lung embryonic fibroblast cDNA library (Miki, T. et al., Gene 83: 137-146, 1989), thereby to obtain a full-length gene having the L668 cDNA sequence. Two full-length genes were obtained from the human lung embryonic fibroblast cDNA library; one gene is a full-length PIG16 cDNA clone in which the 1682-bp fragment was inserted into the pCEV-LAC vector, and then deposited with Accession No. AY305873 into the GenBank database of U.S. NIH on May 24, 2003 (Publication Date: Dec. 31, 2004).

The PIG16 clone inserted into the λ pCEV vector was cleaved by the restriction enzyme NotI and isolated from the phage in the form of ampicillin-resistant pCEV-LAC phagemid vector (see the above reference).

The pCEV-LAC vector containing the PIG16 gene was ligated by T4 DNA ligase to obtain PIG16 plasmid DNA, and then E. coli DH5 α was transformed with the ligated clone.

A 1682-bp full-length sequence of the PIG16 was set forth in SEQ ID NO: 17.

In the DNA sequence of SEQ ID NO: 17, it is estimated that a full-length open reading frame of the protooncogene of the present invention corresponds to nucleotide sequence positions from 696 to 1577, and encodes a protein consisting of 293 amino acids of SEQ ID NO: 18.

6-6: PIG17

The ³²P-labeled L211 was used as the probe to screen a bacteriophage λ gt11 human lung embryonic fibroblast cDNA library (Miki, T. et al., Gene 83: 137-146, 1989), thereby to obtain a full-length gene having the L211 cDNA sequence. Two full-length genes were obtained from the human lung embryonic fibroblast cDNA library; one gene is a full-length PIG17 cDNA clone in which the 626-bp fragment was inserted into the pCEV-LAC vector, and then deposited with Accession No. AY336092 into the GenBank database of U.S. NIH on Jul. 4, 2003 (Publication Date: Dec. 31, 2004).

The PIG5 clone inserted into the λ pCEV vector was cleaved by the restriction enzyme NotI and isolated from the phage in the form of ampicillin-resistant pCEV-LAC phagemid vector (see the above reference).

The pCEV-LAC vector containing the PIG17 gene was ligated by T4 DNA ligase to obtain PIG17 plasmid DNA, and then E. coli DH5 α was transformed with the ligated clone.

A 626-bp full-length sequence of the PIG17 was set forth in SEQ ID NO: 21.

In the DNA sequence of SEQ ID NO: 21, it is estimated that a full-length open reading frame of the protooncogene of the present invention corresponds to nucleotide sequence positions from 59 to 610, and encodes a protein consisting of 183 amino acids of SEQ ID NO: 22.

6-7: PIG19

The ³²P-labeled L722 was used as the probe to screen a bacteriophage λ gt11 human lung embryonic fibroblast cDNA library (Miki, T. et al., Gene 83: 137-146, 1989), thereby to obtain a full-length gene having the L722 cDNA sequence. Two full-length genes were obtained from the human lung embryonic fibroblast cDNA library; one gene is a full-length PIG19 cDNA clone in which the 1031-bp fragment was inserted into the pCEV-LAC vector, and then deposited with Accession No. AY423727 into the GenBank database of U.S. NIH on Sep. 26, 2003 (Publication Date: Dec. 31, 2004).

The PIG19 clone inserted into the λ pCEV vector was cleaved by the restriction enzyme NotI and isolated from the phage in the form of ampicillin-resistant pCEV-LAC phagemid vector (see the above reference).

The pCEV-LAC vector containing the PIG19 gene was ligated by T4 DNA ligase to obtain PIG19 plasmid DNA, and then E. coli DH5 α was transformed with the ligated clone.

A 1031-bp full-length sequence of the PIG19 was set forth in SEQ ID NO: 25.

In the DNA sequence of SEQ ID NO: 25, it is estimated that a full-length open reading frame of the protooncogene of the present invention corresponds to nucleotide sequence positions from 32 to 1030, and encodes a protein consisting of 332 amino acids of SEQ ID NO: 26.

6-8: PIG20

The ³²P-labeled L752 was used as the probe to screen a bacteriophage λ gt11 human lung embryonic fibroblast cDNA library (Miki, T. et al., Gene 83: 137-146, 1989), thereby to obtain a full-length gene having the L752 cDNA sequence. Two full-length genes were obtained from the human lung embryonic fibroblast cDNA library; one gene is a full-length PIG20 cDNA clone in which the 526-bp fragment was inserted into the pCEV-LAC vector, and then deposited with Accession No. AY423728 into the GenBank database of U.S. NIH on Sep. 27, 2003 (Publication Date: Dec. 31, 2004).

The PIG21 clone inserted into the λ pCEV vector was cleaved by the restriction enzyme NotI and isolated from the phage in the form of ampicillin-resistant pCEV-LAC phagemid vector (see the above reference).

The pCEV-LAC vector containing the PIG20 gene was ligated by T4 DNA ligase to obtain PIG20 plasmid DNA, and then E. coli DH5 α was transformed with the ligated clone.

A 526-bp full-length sequence of the PIG20 was set forth in SEQ ID NO: 29.

In the DNA sequence of SEQ ID NO: 29, it is estimated that a full-length open reading frame of the protooncogene of the present invention corresponds to nucleotide sequence positions from 1 to 498, and encodes a protein consisting of 165 amino acids of SEQ ID NO: 30.

6-9: PIG21

The ³²P-labeled L1003 was used as the probe to screen a bacteriophage λ gt11 human lung embryonic fibroblast cDNA library (Miki, T. et al., Gene 83: 137-146, 1989), thereby to obtain a full-length gene having the L1003 cDNA sequence. Two full-length genes were obtained from the human lung embryonic fibroblast cDNA library; one gene is a full-length PIG21 cDNA clone in which the 965-bp fragment was inserted into the pCEV-LAC vector, and then deposited with Accession No. AY336089 into the GenBank database of U.S. NIH on Jul. 4, 2003 (Publication Date: Dec. 31, 2004).

The PIG21 clone inserted into the λ pCEV vector was cleaved by the restriction enzyme NotI and isolated from the phage in the form of ampicillin-resistant pCEV-LAC phagemid vector (see the above reference).

The pCEV-LAC vector containing the PIG21 gene was ligated by T4 DNA ligase to obtain PIG21 plasmid DNA, and then E. coli DH5 α was transformed with the ligated clone.

A 965-bp full-length sequence of the PIG21 was set forth in SEQ ID NO: 33.

In the DNA sequence of SEQ ID NO: 33, it is estimated that a full-length open reading frame of the protooncogene of the present invention corresponds to nucleotide sequence positions from 146 to 961, and encodes a protein consisting of 271 amino acids of SEQ ID NO: 34.

6-10: HCCRBP2

The clone obtained in Example 1 was sequenced according to a dideoxy chain termination method using the Sequenase version 2.0 DNA sequencing kit (United States Biochemical) according to the conventional method. As a result, it was confirmed that a 626-bp protooncogene set forth in SEQ ID NO: 37 was present in the clone, and the clone was named HCCRBP2. A full-length HCCRBP2 cDNA, into which the protooncogene was inserted, was then deposited with Accession No. AY323819 into the GenBank database of U.S. NIH on Jun. 14, 2003.

In the DNA sequence of SEQ ID NO: 37, it is estimated that a full-length open reading frame of the protooncogene HCCRBP2 of the present invention corresponds to nucleotide sequence positions from 9 to 356, and encodes a protein consisting of 115 amino acids of SEQ ID NO: 38.

The HCCRBP2 gene was ligated into a cloning site of a pGEM-T-easy vector (Promega) with T4 DNA ligase to obtain an HCCRBP2 expression plasmid DNA, and then E. coli DH5 α (Stratagene) was transformed with the resultant plasmid.

6-11: TRG2

The ³²P-labeled HP90-811 was used as the probe to screen a bacteriophage λ gt11 human lung embryonic fibroblast cDNA library (Miki, T. et al., Gene 83: 137-146, 1989). A full-length TRG2 cDNA clone, in which the 2302-bp fragment was inserted into the pCEV-LAC vector, was obtained from the human lung embryonic fibroblast cDNA library, and then deposited with Accession No. AY170823 into the GenBank database of U.S. NIH on Oct. 30, 2002 (Publication Date: Dec. 31, 2004).

The TRG2 clone inserted into the λ pCEV vector was cleaved by the restriction enzyme NotI and isolated from the phage in the form of ampicillin-resistant pCEV-LAC phagemid vector (Miki, T. et al., Gene 83: 137-146, 1989).

The pCEV-LAC vector containing the TRG2 gene was ligated by T4 DNA ligase to obtain TRG2 plasmid DNA, and then E. coli DH5 α was transformed with the ligated clone.

A 2302-bp full-length sequence of the TRG2 was set forth in SEQ ID NO: 39.

In the DNA sequence of SEQ ID NO: 39, it is estimated that a full-length open reading frame of the protooncogene of the present invention corresponds to nucleotide sequence positions from 747 to 2066, and encodes a protein consisting of 439 amino acids of SEQ ID NO: 40.

### EXAMPLE 7

### Northern Blotting Analysis of Genes in Various Cells

7-1: PIG5, PIG11, PIG16, PIG17, PIG19, PIG20, PIG21

The total RNA samples were extracted from the normal lung tissue, the left lung cancer tissue, the metastatic lung cancer tissue metastasized from the left lung to the right lung, and the A549, NCI-H2009 (American Type Culture Collection; ATCC Number CRL-5911) and NCI-H441 (American Type Culture Collection; ATCC Number HTB-174) lung cancer cell lines in the same manner as in Example 1.

In order to determine an expression level of each of the PIG5; PIG11; PIG16; PIG17; PIG19; PIG20; and PIG21 genes, 20 μg of each of the total denatured RNA samples extracted from each of the tissues and the cell lines was electrophoresized in an 1% formaldehyde agarose gel, and then the resultant agarose gel was transferred to a nylon membrane ((Boehringer-Mannheim, Germany). The blot was then hybridized with each of the ³²P-labeled and randomly primed partial cDNA proves of the L699; L667; L668; L211; L722; L752; and L1003 genes prepared using the Rediprime II random prime labelling system ((Amersham, United Kingdom). The northern blotting analysis was repeated twice, and therefore the resultant blots were quantitified with the densitometer and normalized with the β-actin.

FIG. 11A shows a northern blotting result to determine whether or not the PIG5 protooncogene is expressed in the normal lung tissue, the lung cancer tissue, the metastatic lung cancer tissue and the lung cancer cell lines (A549, NCI-H2009, and NCI-H441). As shown in FIG. 11A, it was revealed that the expression level of the PIG5 protooncogene was significantly increased in the lung cancer tissue, the metastatic lung cancer tissue and the A549, NCI-H2009 and NCI-H441 lung cancer cell lines, but very low or not detected in the normal lung tissue. In FIG. 11, Lane “Normal” represents the normal lung tissue, Lane “Cancer” represents the lung cancer tissue, Lane “metastasis” represents the metastatic lung cancer tissue, and each of Lanes “A549”, “NCI-H2009” and “NCI-H441” represents the lung cancer cell line. FIG. 11(b) shows the northern blotting result indicating presence of mRNA transcript by hybridizing the same sample with β-actin probe.

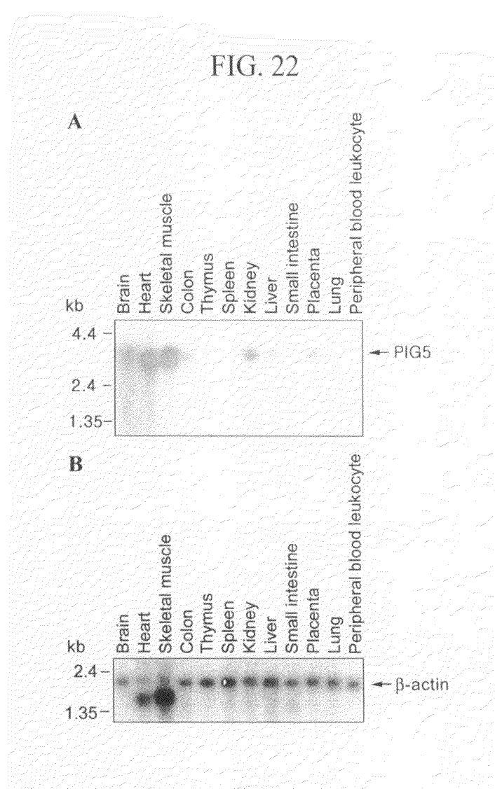

FIG. 22(a) shows a northern blotting result to determine whether or not the PIG5 protooncogene is expressed in the normal human 12-lane multiple tissues (Clontech), for example brain, heart, striated muscle, large intestines, thymus, spleen, kidneys, liver, small intestines, placenta, lungs and peripheral blood leukocyte. FIG. 22(b) shows the northern blotting result indicating presence of mRNA transcript by hybridizing the same sample with β-actin probe. As shown in FIG. 22(a), it was revealed that the PIG5 mRNA transcript (approximately 3.0 kb) was weakly expressed in the muscle tissue and the heart tissue, and very weakly expressed or not expressed in the other normal tissues.

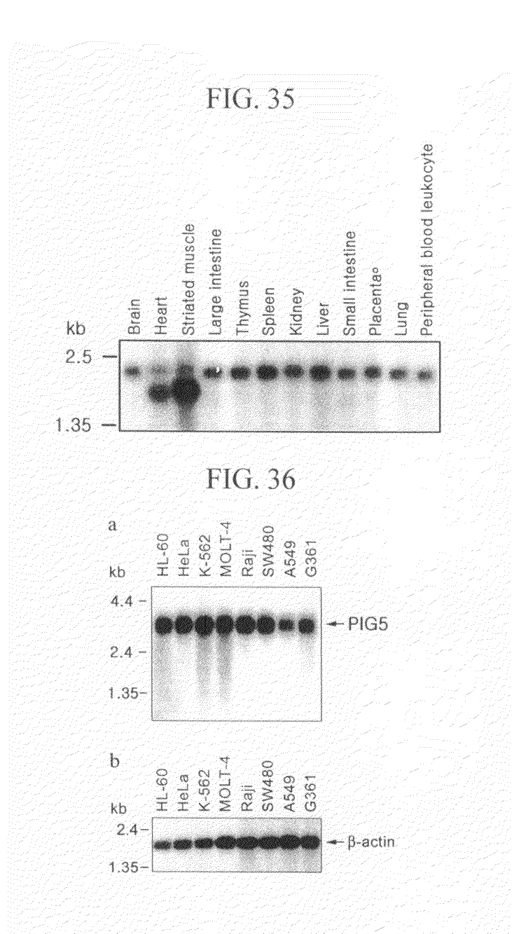

FIG. 36(a) shows a northern blotting result to determine whether or not the PIG5 protooncogene is expressed in the human cancer cell lines, for example HL-60, HeLa, K-562, MOLT-4, Raji, SW480, A549 and G361 (Clontech). FIG. 36(b) shows the northern blotting result indicating presence of mRNA transcript by hybridizing the same sample with β-actin probe. As shown in FIG. 36(a), it was revealed that the PIG5 protooncogene was very highly expressed in the promyelocyte leukemia cell line HL-60, the HeLa uterine cancer cell line, the chronic myelogenous leukemia cell line K-562, the lymphoblastic leukaemia cell line MOLT-4, the Burkitt lymphoma cell line Raji, the colon cancer cell line SW480, the lung cancer cell line A549 and the skin cancer cell line G361.

FIG. 14(a) shows a northern blotting result to determine whether or not the PIG11 protooncogene is expressed in the normal lung tissue, the lung cancer tissue, the metastatic lung cancer tissue and the lung cancer cell lines (A549, NCI-H2009, and NCI-H441). As shown in FIG. 14(a), it was revealed that the expression level of the PIG11 protooncogene was significantly increased in the lung cancer tissue, the metastatic lung cancer tissue and the A549, NCI-H2009 and NCI-H441 lung cancer cell lines, but very low or not detected in the normal lung tissue. In FIG. 14, Lane “Normal” represents the normal lung tissue, Lane “Cancer” represents the lung cancer tissue, Lane “metastasis” represents the metastatic lung cancer tissue, and each of Lanes “A549”, “NCI-H2009” and “NCI-H441” represents the lung cancer cell line. FIG. 14(b) shows the northern blotting result indicating presence of mRNA transcript by hybridizing the same sample with β-actin probe.

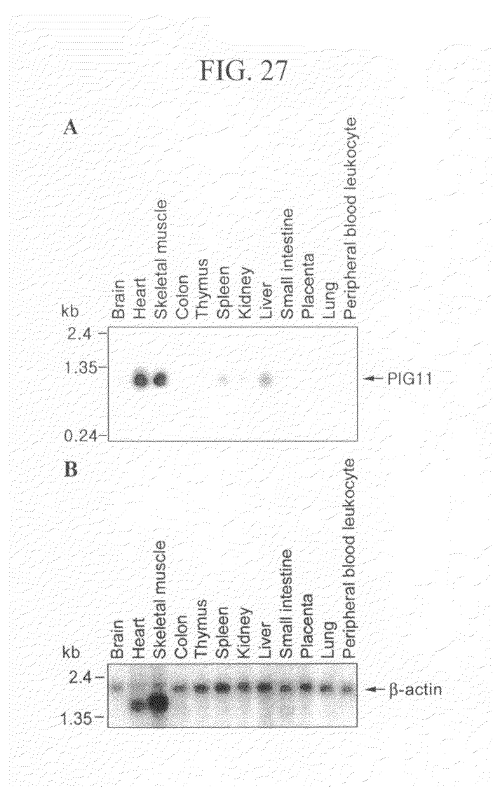

FIG. 27(a) shows a northern blotting result to determine whether or not the PIG11 protooncogene is expressed in the normal human 12-lane multiple tissues (Clontech), for example brain, heart, striated muscle, large intestines, thymus, spleen, kidneys, liver, small intestines, placenta, lungs and peripheral blood leukocyte. FIG. 27(b) shows the northern blotting result indicating presence of mRNA transcript by hybridizing the same sample with β-actin probe. As shown in FIG. 27(a), it was revealed that the PIG11 mRNA transcript (approximately 1.3 kb) was weakly expressed in the muscle tissue and the heart tissue, and very weakly expressed or not expressed in the other normal tissues.

FIG. 41(a) shows a northern blotting result to determine whether or not the PIG11 protooncogene is expressed in the human cancer cell lines, for example HL-60, HeLa, K-562, MOLT-4, Raji, SW480, A549 and G361 (Clontech). FIG. 41(b) shows the northern blotting result indicating presence of mRNA transcript by hybridizing the same sample with β-actin probe. As shown in FIG. 41(a), it was revealed that the PIG11 protooncogene was very highly expressed in the promyelocyte leukemia cell line HL-60, the HeLa uterine cancer cell line, the chronic myelogenous leukemia cell line K-562, the lymphoblastic leukaemia cell line MOLT-4, the Burkitt lymphoma cell line Raji, the colon cancer cell line SW480, the lung cancer cell line A549 and the skin cancer cell line G361.

FIG. 15(a) shows a northern blotting result to determine whether or not the PIG16 protooncogene is expressed in the normal lung tissue, the lung cancer tissue, the metastatic lung cancer tissue and the lung cancer cell lines (A549, NCI-H2009, and NCI-H441). As shown in FIG. 15(a), it was revealed that the expression level of the PIG16 protooncogene was significantly increased in the lung cancer tissue, the metastatic lung cancer tissue and the A549, NCI-H2009 and NCI-H441 lung cancer cell lines, but very low or not detected in the normal lung tissue. In FIG. 15, Lane “Normal” represents the normal lung tissue, Lane “Cancer” represents the lung cancer tissue, Lane “metastasis” represents the metastatic lung cancer tissue, and each of Lanes “A549”, “NCI-H2009” and “NCI-H441” represents the lung cancer cell line. FIG. 15(b) shows the northern blotting result indicating presence of mRNA transcript by hybridizing the same sample with β-actin probe.

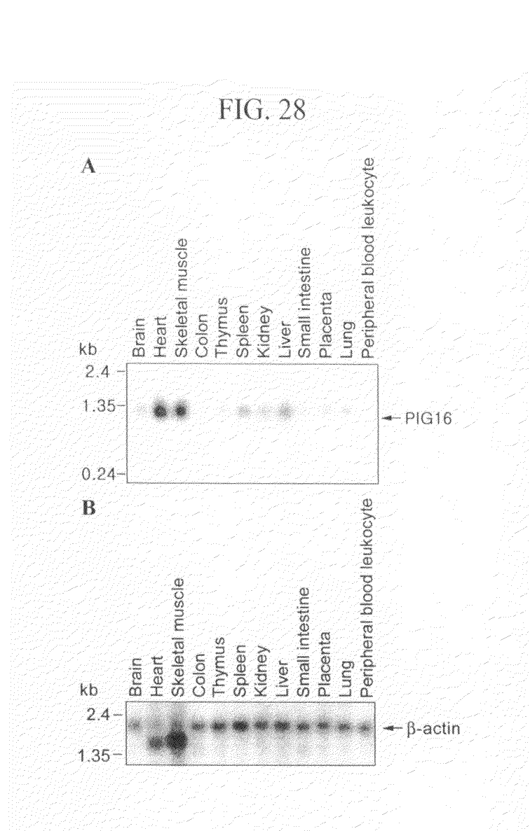

FIG. 28(a) shows a northern blotting result to determine whether or not the PIG16 protooncogene is expressed in the normal human 12-lane multiple tissues (Clontech), for example brain, heart, striated muscle, large intestines, thymus, spleen, kidneys, liver, small intestines, placenta, lungs and peripheral blood leukocyte. FIG. 28(b) shows the northern blotting result indicating presence of mRNA transcript by hybridizing the same sample with β-actin probe. As shown in FIG. 28(a), it was revealed that the PIG16 mRNA transcript (approximately 1.3 kb) was weakly expressed in the muscle tissue and the heart tissue, and very weakly expressed or not expressed in the other normal tissues.

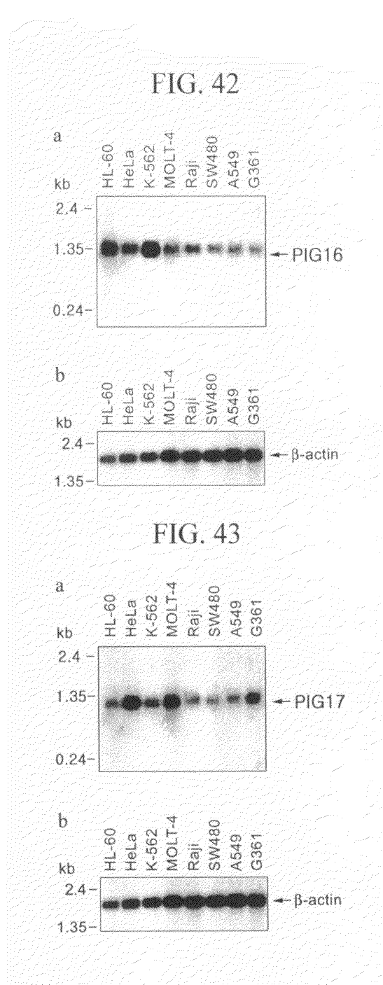

FIG. 42(a) shows a northern blotting result to determine whether or not the PIG16 protooncogene is expressed in the human cancer cell lines, for example HL-60, HeLa, K-562, MOLT-4, Raji, SW480, A549 and G361 (Clontech). FIG. 42(b) shows the northern blotting result indicating presence of mRNA transcript by hybridizing the same sample with β-actin probe. As shown in FIG. 42(a), it was revealed that the PIG16 protooncogene was very highly expressed in the promyelocyte leukemia cell line HL-60, the HeLa uterine cancer cell line, the chronic myelogenous leukemia cell line K-562, the lymphoblastic leukaemia cell line MOLT-4, the Burkitt lymphoma cell line Raji, the colon cancer cell line SW480, the lung cancer cell line A549 and the skin cancer cell line G361.

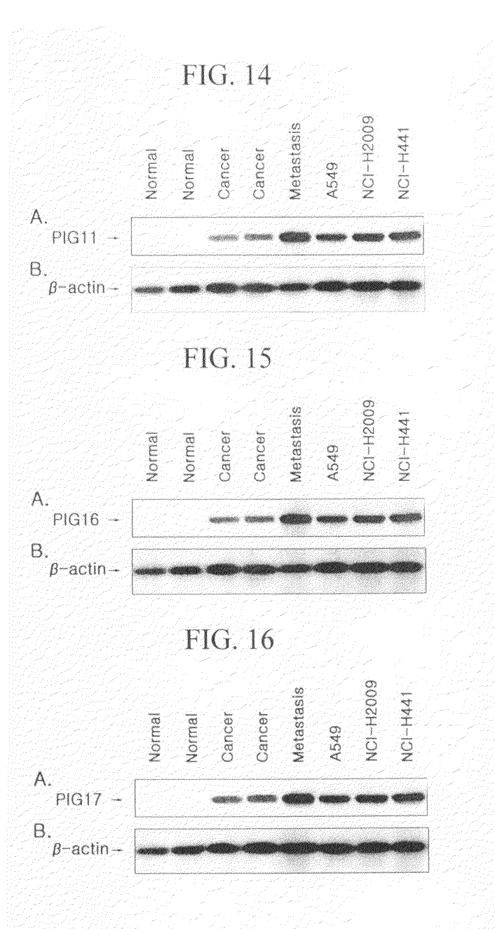

FIG. 16(a) shows a northern blotting result to determine whether or not the PIG17 protooncogene is expressed in the normal lung tissue, the lung cancer tissue, the metastatic lung cancer tissue and the lung cancer cell lines (A549, NCI-H2009, and NCI-H441). As shown in FIG. 16(a), it was revealed that the expression level of the PIG17 protooncogene was significantly increased in the lung cancer tissue, the metastatic lung cancer tissue and the A549, NCI-H2009 and NCI-H441 lung cancer cell lines, but very low or not detected in the normal lung tissue. In FIG. 16, Lane “Normal” represents the normal lung tissue, Lane “Cancer” represents the lung cancer tissue, Lane “metastasis” represents the metastatic lung cancer tissue, and each of Lanes “A549”, “NCI-H2009” and “NCI-H441” represents the lung cancer cell line. FIG. 16(b) shows the northern blotting result indicating presence of mRNA transcript by hybridizing the same sample with β-actin probe.

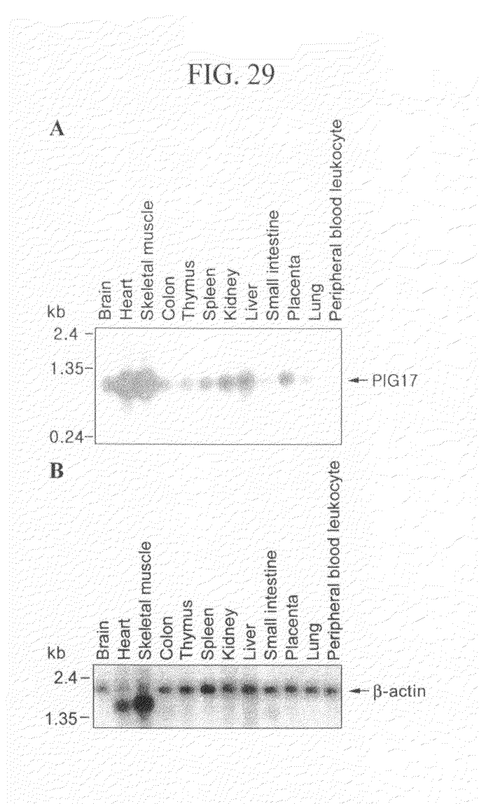

FIG. 29(a) shows a northern blotting result to determine whether or not the PIG17 protooncogene is expressed in the normal human 12-lane multiple tissues (Clontech), for example brain, heart, striated muscle, large intestines, thymus, spleen, kidneys, liver, small intestines, placenta, lungs and peripheral blood leukocyte. FIG. 29(b) shows the northern blotting result indicating presence of mRNA transcript by hybridizing the same sample with β-actin probe. As shown in FIG. 29(a), it was revealed that the PIG17 mRNA transcript (approximately 1.3 kb) was weakly expressed in the muscle tissue and the heart tissue, and very weakly expressed or not expressed in the other normal tissues.

FIG. 43(a) shows a northern blotting result to determine whether or not the PIG17 protooncogene is expressed in the human cancer cell lines, for example HL-60, HeLa, K-562, MOLT-4, Raji, SW480, A549 and G361 (Clontech). FIG. 43(b) shows the northern blotting result indicating presence of mRNA transcript by hybridizing the same sample with β-actin probe. As shown in FIG. 43(a), it was revealed that the PIG17 protooncogene was very highly expressed in the promyelocyte leukemia cell line HL-60, the HeLa uterine cancer cell line, the chronic myelogenous leukemia cell line K-562, the lymphoblastic leukaemia cell line MOLT-4, the Burkitt lymphoma cell line Raji, the colon cancer cell line SW480, the lung cancer cell line A549 and the skin cancer cell line G361.

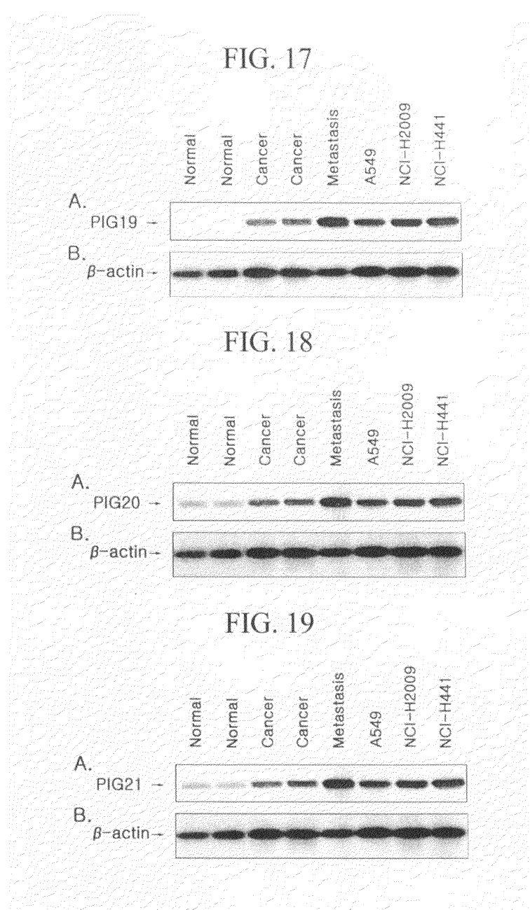

FIG. 17(a) shows a northern blotting result to determine whether or not the PIG19 protooncogene is expressed in the normal lung tissue, the lung cancer tissue, the metastatic lung cancer tissue and the lung cancer cell lines (A549, NCI-H2009, and NCI-H441). As shown in FIG. 17(a), it was revealed that the expression level of the PIG19 protooncogene was significantly increased in the lung cancer tissue, the metastatic lung cancer tissue and the A549, NCI-H2009 and NCI-H441 lung cancer cell lines, but very low or not detected in the normal lung tissue. In FIG. 17, Lane “Normal” represents the normal lung tissue, Lane “Cancer” represents the lung cancer tissue, Lane “metastasis” represents the metastatic lung cancer tissue, and each of Lanes “A549”, “NCI-H2009” and “NCI-H441” represents the lung cancer cell line. FIG. 17(b) shows the northern blotting result indicating presence of mRNA transcript by hybridizing the same sample with β-actin probe.

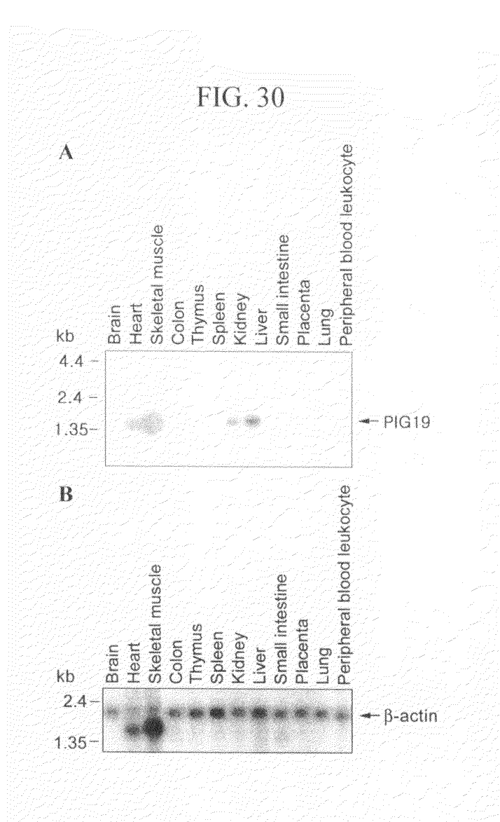

FIG. 30(a) shows a northern blotting result to determine whether or not the PIG19 protooncogene is expressed in the normal human 12-lane multiple tissues (Clontech), for example brain, heart, striated muscle, large intestines, thymus, spleen, kidneys, liver, small intestines, placenta, lungs and peripheral blood leukocyte. FIG. 30(b) shows the northern blotting result indicating presence of mRNA transcript by hybridizing the same sample with β-actin probe. As shown in FIG. 30(a), it was revealed that the PIG19 mRNA transcript (approximately 1.5 kb) was weakly expressed in the muscle tissue and the heart tissue, and very weakly expressed or not expressed in the other normal tissues.

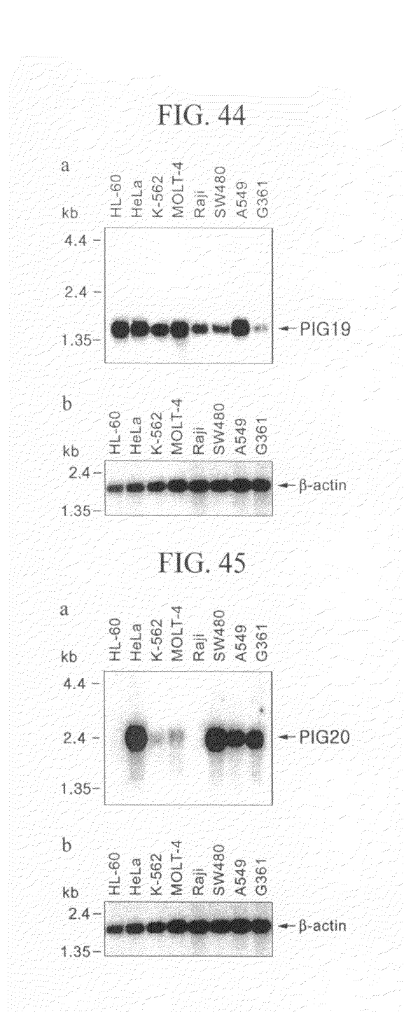

FIG. 44(a) shows a northern blotting result to determine whether or not the PIG19 protooncogene is expressed in the human cancer cell lines, for example HL-60, HeLa, K-562, MOLT-4, Raji, SW480, A549 and G361 (Clontech). FIG. 44(b) shows the northern blotting result indicating presence of mRNA transcript by hybridizing the same sample with β-actin probe. As shown in FIG. 44(a), it was revealed that the PIG19 protooncogene was very highly expressed in the promyelocyte leukemia cell line HL-60, the HeLa uterine cancer cell line, the chronic myelogenous leukemia cell line K-562, the lymphoblastic leukaemia cell line MOLT-4, the Burkitt lymphoma cell line Raji, the colon cancer cell line SW480, the lung cancer cell line A549 and the skin cancer cell line G361.

FIG. 18(a) shows a northern blotting result to determine whether or not the PIG20 protooncogene is expressed in the normal lung tissue, the lung cancer tissue, the metastatic lung cancer tissue and the lung cancer cell lines (A549, NCI-H2009, and NCI-H441). As shown in FIG. 18(a), it was revealed that the expression level of the PIG20 protooncogene was significantly increased in the lung cancer tissue, the metastatic lung cancer tissue and the A549, NCI-H2009 and NCI-H441 lung cancer cell lines, but very low or not detected in the normal lung tissue. In FIG. 18, Lane “Normal” represents the normal lung tissue, Lane “Cancer” represents the lung cancer tissue, Lane “metastasis” represents the metastatic lung cancer tissue, and each of Lanes “A549”, “NCI-H2009” and “NCI-H441” represents the lung cancer cell line. FIG. 18(b) shows the northern blotting result indicating presence of mRNA transcript by hybridizing the same sample with β-actin probe.

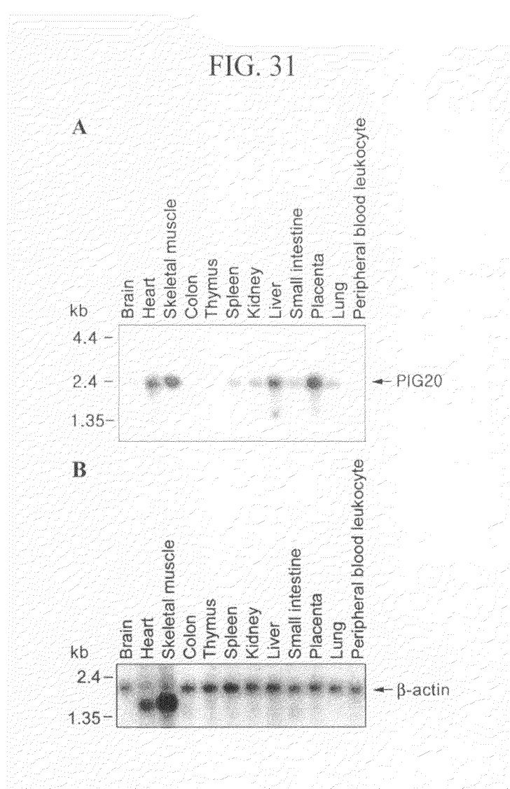

FIG. 31(a) shows a northern blotting result to determine whether or not the PIG20 protooncogene is expressed in the normal human 12-lane multiple tissues (Clontech), for example brain, heart, striated muscle, large intestines, thymus, spleen, kidneys, liver, small intestines, placenta, lungs and peripheral blood leukocyte. FIG. 31(b) shows the northern blotting result indicating presence of mRNA transcript by hybridizing the same sample with β-actin probe. As shown in FIG. 31(a), it was revealed that the PIG20 mRNA transcript (approximately 2.5 kb) was weakly expressed in the muscle tissue, the heart tissue and the placenta tissue, and very weakly expressed or not expressed in the other normal tissues.

FIG. 45(a) shows a northern blotting result to determine whether or not the PIG20 protooncogene is expressed in the human cancer cell lines, for example HL-60, HeLa, K-562, MOLT-4, Raji, SW480, A549 and G361 (Clontech). FIG. 45(b) shows the northern blotting result indicating presence of mRNA transcript by hybridizing the same sample with β-actin probe. As shown in FIG. 45(a), it was revealed that the PIG20 protooncogene was very highly expressed in the HeLa uterine cancer cell line, the colon cancer cell line SW480, the lung cancer cell line A549 and the skin cancer cell line G361, but very slightly expressed or not expressed in the promyelocyte leukemia cell line HL-60, the chronic myelogenous leukemia cell line K-562, the lymphoblastic leukaemia cell line MOLT-4 and the Burkitt lymphoma cell line Raji.

FIG. 19(a) shows a northern blotting result to determine whether or not the PIG21 protooncogene is expressed in the normal lung tissue, the lung cancer tissue, the metastatic lung cancer tissue and the lung cancer cell lines (A549, NCI-H2009, and NCI-H441). As shown in FIG. 119(a), it was revealed that the expression level of the PIG21 protooncogene was significantly increased in the lung cancer tissue, the metastatic lung cancer tissue and the A549, NCI-H2009 and NCI-H441 lung cancer cell lines, but very low in the normal lung tissue. In FIGS. 19(a) and (b), Lane “Normal” represents the normal lung tissue, Lane “Cancer” represents the lung cancer tissue, Lane “metastasis” represents the metastatic lung cancer tissue, and each of Lanes “A549”, “NCI-H2009” and “NCI-H441” represents the lung cancer cell line. FIG. 19(b) shows the northern blotting result indicating presence of mRNA transcript by hybridizing the same sample with β-actin probe.

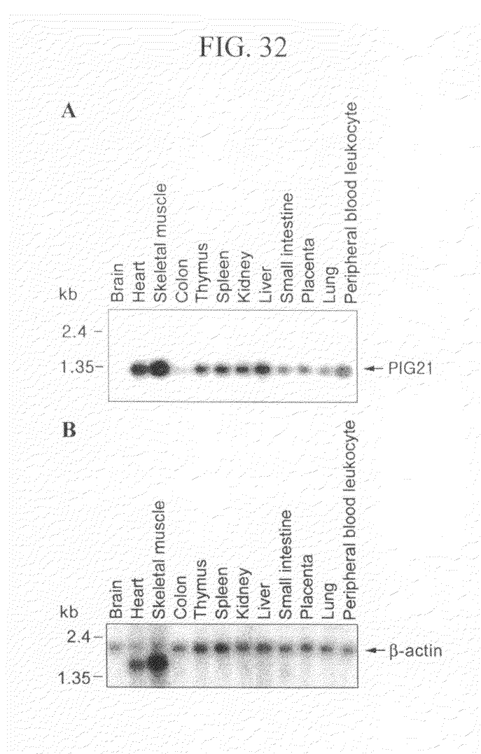

FIG. 32(a) shows a northern blotting result to determine whether or not the PIG21 protooncogene is expressed in the normal human 12-lane multiple tissues (Clontech), for example brain, heart, striated muscle, large intestines, thymus, spleen, kidneys, liver, small intestines, placenta, lungs and peripheral blood leukocyte. FIG. 32(b) shows the northern blotting result indicating presence of mRNA transcript by hybridizing the same sample with β-actin probe. As shown in FIG. 32(a), it was revealed that the PIG21 mRNA transcript (approximately 1.3 kb) was weakly expressed in the muscle tissue and the heart tissue, and very weakly expressed or not expressed in the other normal tissues.

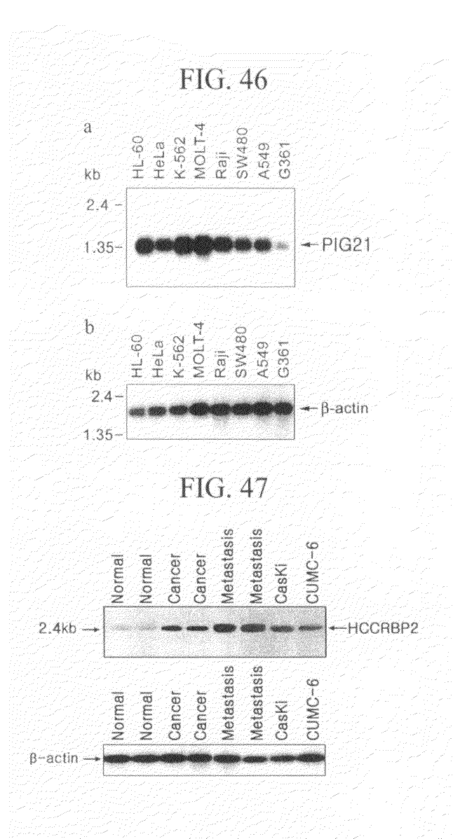

FIG. 46(a) shows a northern blotting result to determine whether or not the PIG21 protooncogene is expressed in the human cancer cell lines, for example HL-60, HeLa, K-562, MOLT-4, Raji, SW480, A549 and G361 (Clontech). FIG. 46(b) shows the northern blotting result indicating presence of mRNA transcript by hybridizing the same sample with β-actin probe. As shown in FIG. 45(a), it was revealed that the PIG21 protooncogene was very highly expressed in the promyelocyte leukemia cell line HL-60, the HeLa uterine cancer cell line, the chronic myelogenous leukemia cell line K-562, the lymphoblastic leukaemia cell line MOLT-4, the Burkitt lymphoma cell line Raji, the colon cancer cell line SW480, the lung cancer cell line A549 and the skin cancer cell line G361.

7-2: PIG6, PIG7, TRG2

The total RNA samples were extracted from the normal exocervical tissue, the cervical cancer tissue, the metastatic cervical lymph node tissue and the cervical cancer cell lines CaSki (ATCC CRL 1550) and CUMC-6 in the same manner as in Example 1.

In order to determine an expression level of each of the PIG6; PIG7 and TRG2 genes, 20 μg of each of the total denatured RNA samples extracted from each of the tissues and cell lines was electrophoresized in an 1% formaldehyde agarose gel, and then the resultant agarose gel was transferred to a nylon membrane ((Boehringer-Mannheim, Germany). The blot was then hybridized with the ³²P-labeled and randomly primed full-length PIG cDNA probes prepared using the Rediprime II random prime labelling system ((Amersham, United Kingdom). The northern blotting analysis was repeated twice, and therefore the resultant blots were quantitified with the densitometer and normalized with the β-actin.

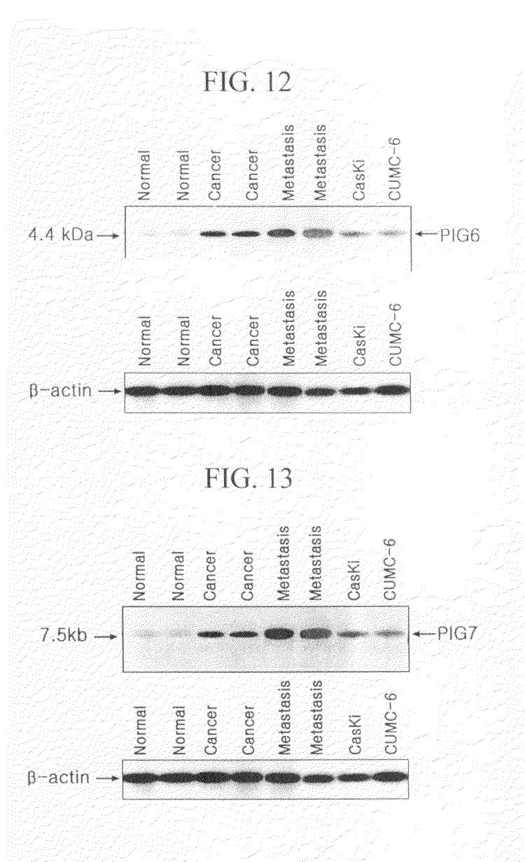

FIG. 12(a) shows a northern blotting result to determine whether or not the PIG6 protooncogene is expressed in the normal exocervical tissue, the cervical cancer tissues, the metastatic cervical lymph node tissue and the cervical cancer cell lines (CaSki and CUMC-6). As shown in FIG. 12(a), it was revealed that the expression level of the PIG6 protooncogene was increased in the cervical cancer tissue and the cervical cancer cell lines CaSki and CUMC-6, that is, dominant PIG6 mRNA transcript of approximately 4.4 kb was overexpressed, and the PIG6 protooncogene was the most highly expressed especially in the metastatic cervical lymph node tissue, but very low expressed in the normal tissue. In FIGS. 12(a) and (b), Lane “Normal” represents the normal exocervical tissue, Lane “Cancer” represents the cervical cancer tissue, Lane “metastasis” represents the metastatic cervical lymph node tissue, and each of Lanes “CaSki” and “CUMC-6” represents the uterine cancer cell line. FIG. 12(b) shows the northern blotting result indicating presence of mRNA transcript by hybridizing the same sample with β-actin probe.

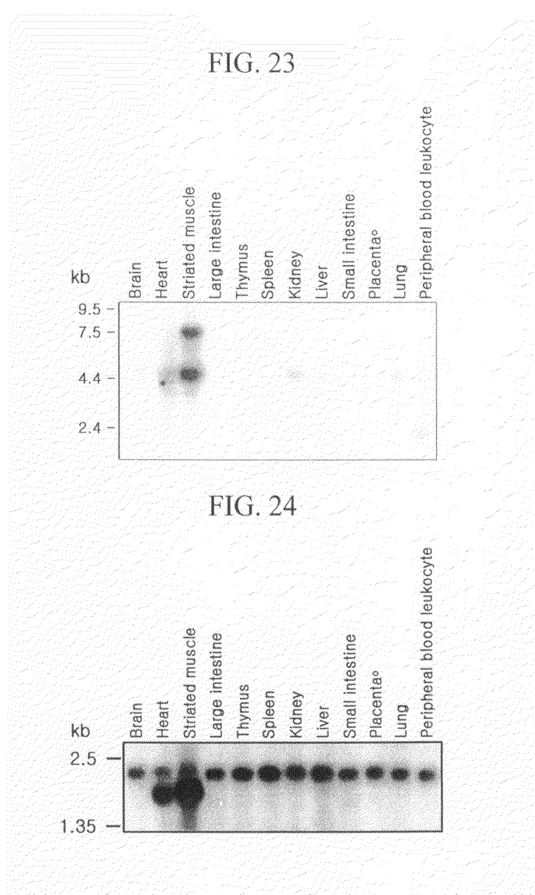

FIG. 23 shows a northern blotting result to determine whether or not the PIG6 protooncogene is expressed in the normal human 12-lane multiple tissues (Clontech), for example brain, heart, striated muscle, large intestines, thymus, spleen, kidneys, liver, small intestines, placenta, lungs and peripheral blood leukocyte tissues. FIG. 24 shows the northern blotting result indicating presence of mRNA transcript by hybridizing the same sample with β-actin probe. As shown in FIG. 23, it was revealed that the PIG6 mRNA transcripts (a dominant PIG6 mRNA transcript of approximately 4.4 kb and an PIG6 mRNA transcript of approximately 8 kb) were weakly expressed in the normal tissues such as brain, heart, striated muscle, large intestines, thymus, spleen, kidneys, liver, small intestines, placenta, lungs and peripheral blood leukocyte.

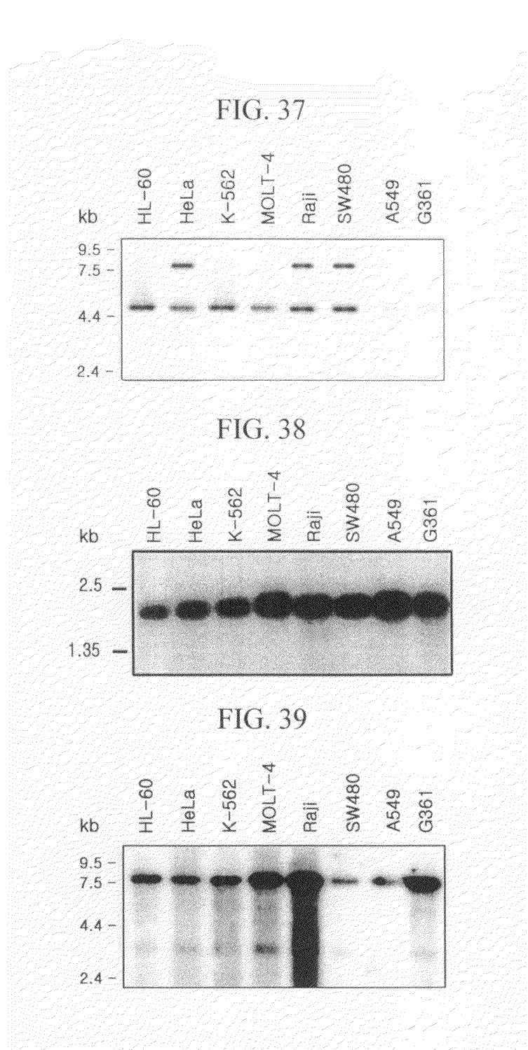

FIG. 37 shows a northern blotting result to determine whether or not the PIG6 protooncogene is expressed in the human cancer cell lines, for example HL-60, HeLa, K-562, MOLT-4, Raji, SW480, A549 and G361 (Clontech). FIG. 38 shows the northern blotting result indicating presence of mRNA transcript by hybridizing the same sample with β-actin probe. As shown in FIG. 37, it was revealed that the PIG6 mRNA transcripts (a dominant PIG6 mRNA transcript of approximately 4.4 kb and an PIG6 mRNA transcript of approximately 8 kb) were very highly expressed in the promyelocyte leukemia cell line HL-60, the HeLa uterine cancer cell line, the chronic myelogenous leukemia cell line K-562, the lymphoblastic leukaemia cell line MOLT-4, the Burkitt lymphoma cell line Raji and the colon cancer cell line SW480.

FIG. 13(a) shows a northern blotting result to determine whether or not the PIG7 protooncogene is expressed in the normal exocervical tissue, the cervical cancer tissue, the metastatic cervical lymph node tissue and the cervical cancer cell lines (CaSki and CUMC-6). As shown in FIG. 13(a), it was revealed that the expression level of the PIG7 protooncogene was increased in the cervical cancer tissue and the cervical cancer cell lines CaSki and CUMC-6, that is, dominant PIG7 mRNA transcript of approximately 7.5 kb was overexpressed, and the PIG7 protooncogene was the most highly expressed especially in the metastatic cervical lymph node tissue, but very low expressed in the normal tissues. In FIGS. 13(a) and (b), Lane “Normal” represents the normal exocervical tissue, Lane “Cancer” represents the cervical cancer tissue, Lane “metastasis” represents the metastatic cervical lymph node tissue, and each of Lanes “CaSki” and “CUMC-6” represents the uterine cancer cell line. FIG. 13(b) shows the northern blotting result indicating presence of mRNA transcript by hybridizing the same sample with β-actin probe.

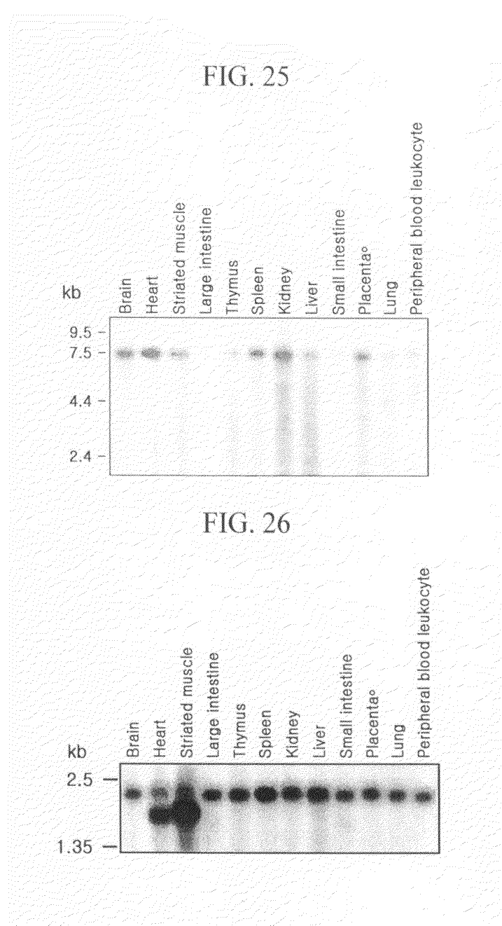

FIG. 25 shows a northern blotting result to determine whether or not the PIG7 protooncogene is expressed in the normal human 12-lane multiple tissues (Clontech), for example brain, heart, striated muscle, large intestines, thymus, spleen, kidneys, liver, small intestines, placenta, lungs and peripheral blood leukocyte tissues. FIG. 26 shows the northern blotting result indicating presence of mRNA transcript by hybridizing the same sample with β-actin probe. As shown in FIG. 25, it was revealed that the PIG7 mRNA transcript (a dominant PIG7 mRNA transcript of approximately 7.5 kb) was weakly expressed in the normal tissues such as brain, heart, striated muscle, large intestines, thymus, spleen, kidneys, liver, small intestines, placenta, lungs and peripheral blood leukocyte.

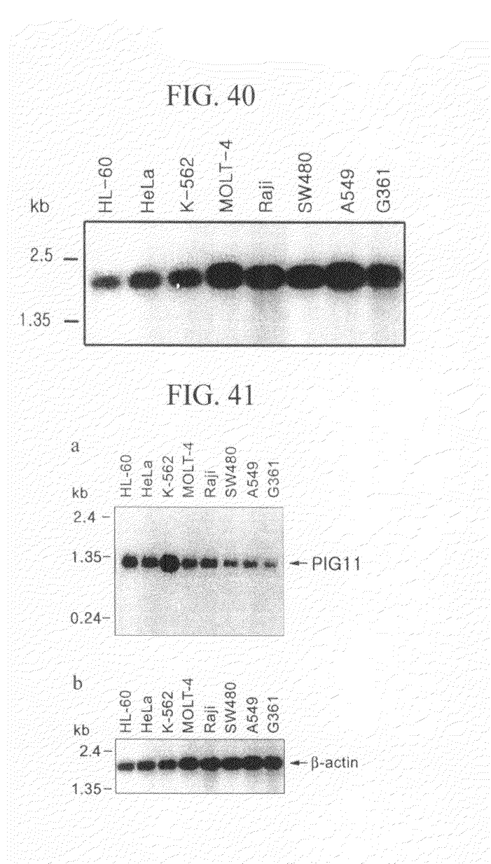

FIG. 39 shows a northern blotting result to determine whether or not the PIG7 protooncogene is expressed in the human cancer cell lines, for example HL-60, HeLa, K-562, MOLT-4, Raji, SW480, A549 and G361 (Clontech). FIG. 40 shows the northern blotting result indicating presence of mRNA transcript by hybridizing the same sample with β-actin probe. As shown in FIG. 39, it was revealed that the PIG7 mRNA transcripts (a dominant PIG7 mRNA transcript of approximately 7.5 kb) were very highly expressed in the promyelocyte leukemia cell line HL-60, the HeLa uterine cancer cell line, the chronic myelogenous leukemia cell line K-562, the lymphoblastic leukaemia cell line MOLT-4, the Burkitt lymphoma cell line Raji, the colon cancer cell line SW480, the lung cancer cell line A549 and the skin cancer cell line G361.

FIG. 21(a) shows a northern blotting result to determine whether or not the TRG2 protooncogene is expressed in the normal exocervical tissue, the cervical cancer tissue, the metastatic cervical lymph node tissue and the cervical cancer cell lines (CaSki and CUMC-6). As shown in FIG. 21(a), it was revealed that the expression level of the TRG2 protooncogene was increased in the cervical cancer tissue and the cervical cancer cell lines CaSki and CUMC-6, that is, dominant TRG2 mRNA transcript of approximately 10 kb was overexpressed. In FIGS. 21(a) and (b), Lane “Normal” represents the normal exocervical tissue, Lane “Cancer” represents the cervical cancer tissue, Lane “metastasis” represents the metastatic cervical lymph node tissue, and each of Lanes “CaSki” and “CUMC-6” represents the uterine cancer cell line. FIG. 21(b) shows the northern blotting result indicating presence of mRNA transcript by hybridizing the same sample with β-actin probe.

FIG. 34 shows a northern blotting result to determine whether or not the TRG2 protooncogene is expressed in the normal human 12-lane multiple tissues (Clontech), for example brain, heart, striated muscle, large intestines, thymus, spleen, kidneys, liver, small intestines, placenta, lungs and peripheral blood leukocyte tissues. FIG. 35 shows the northern blotting result indicating presence of mRNA transcript by hybridizing the same sample with β-actin probe. As shown in FIG. 34, it was revealed that the TRG2 mRNA transcript (a dominant TRG2 mRNA transcript of approximately 10 kb) was expressed in the normal muscle tissue, but very slightly expressed in the normal tissues such as brain, heart, large intestines, thymus, spleen, kidneys, liver, small intestines, placenta, lungs and peripheral blood leukocyte.

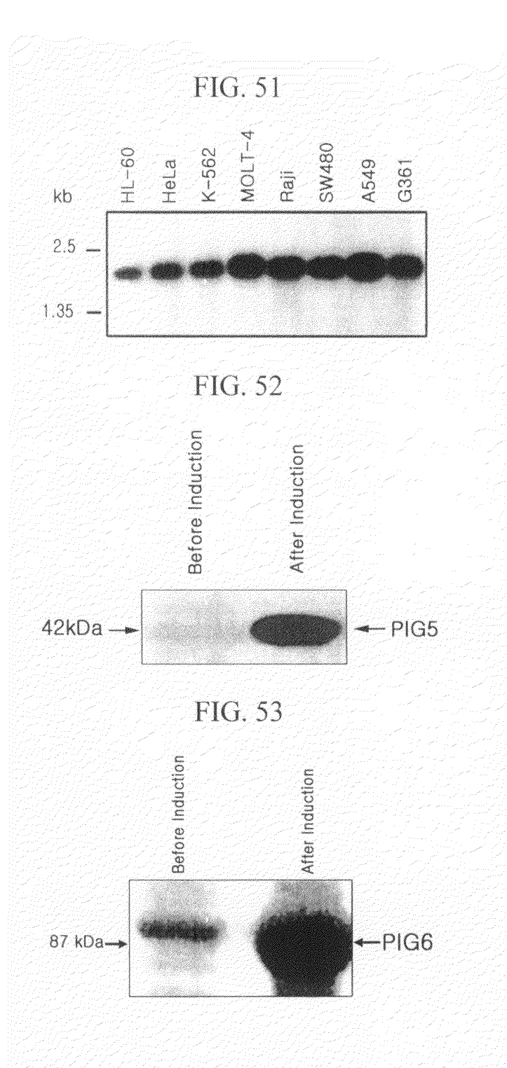

FIG. 50 shows a northern blotting result to determine whether or not the TRG2 protooncogene is expressed in the human cancer cell lines, for example HL-60, HeLa, K-562, MOLT-4, Raji, SW480, A549 and G361 (Clontech). FIG. 51 shows the northern blotting result indicating presence of mRNA transcript by hybridizing the same sample with β-actin probe. As shown in FIG. 50, it was revealed that the TRG2 mRNA transcript (a dominant TRG2 mRNA transcript of approximately 10 kb) was very highly expressed in the HeLa uterine cancer cell line, the chronic myelogenous leukemia cell line K-562, the Burkitt lymphoma cell line Raji, the colon cancer cell line SW480, the lung cancer cell line A549 and the skin cancer cell line G361.

7-3: HCCRBP2

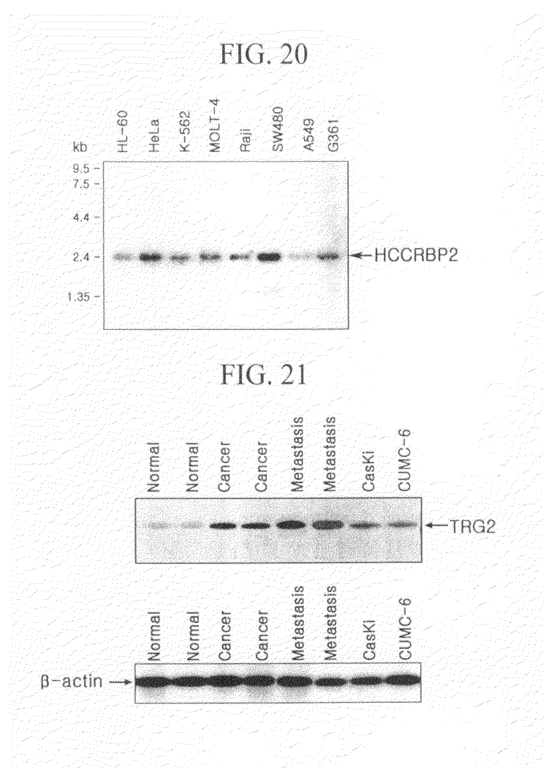

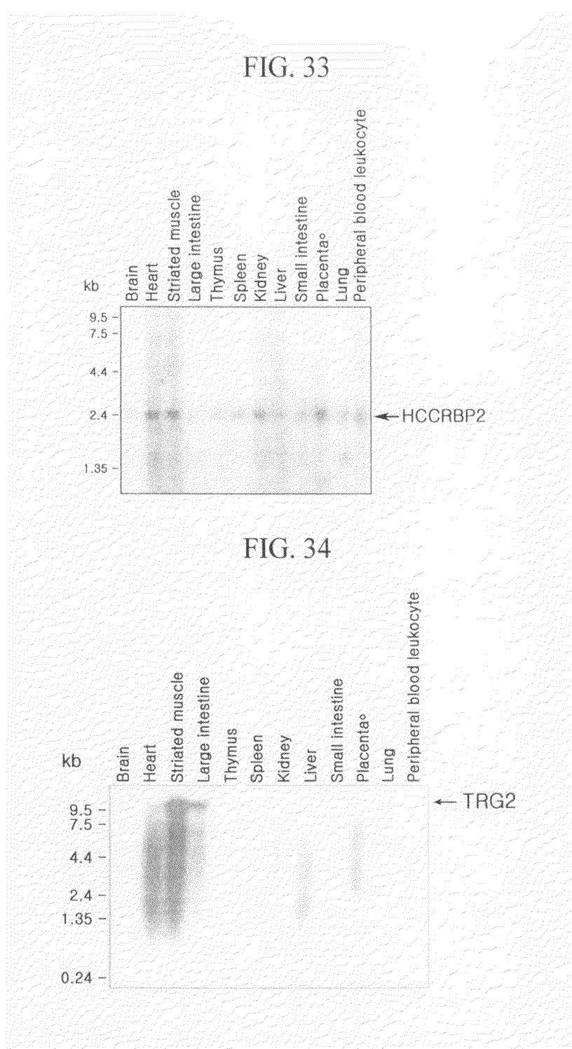

In order to determine an expression level of the HCCRBP2 gene in normal cells and cancer cells, the commercially available normal human 12-lane multiple tissuess (Clontech) blotted on a nylon membrane after each of normal RNA samples was extracted from 12 kinds of organs (FIG. 33); and the commercially available human cancer cell 8-lane multiple tissues (Clontech) blotted on a nylon membrane after each of RNA samples was extracted from 8 kinds of cancer cells (FIG. 20) were purchased and used for comparing their expression levels to each other. On two kinds of the membranes, the blots were then hybridized with the ³²P-labeled and randomly primed full-length HCCRBP2 cDNA probe prepared using the Rediprime II random prime labelling system (Amersham). The northern blotting analysis was repeated twice, and therefore the resultant blots were quantitified with the densitometer and normalized with the β-actin.

FIG. 33 shows a northern blotting result to determine whether or not the HCCRBP2 protooncogene is expressed in the normal human 12-lane multiple tissues (Clontech), for example brain, heart, striated muscle, large intestines, thymus, spleen, kidneys, liver, small intestines, placenta, lungs and peripheral blood leukocyte tissues. FIG. 33 shows the northern blotting result indicating presence of mRNA transcript by hybridizing the same sample with β-actin probe. As shown in FIG. 33, it was revealed that the HCCRBP2 mRNA transcripts (a 2.4-kb transcript) was weakly expressed or not expressed in the various normal tissues.

FIG. 20 shows a northern blotting result to determine whether or not the HCCRBP2 protooncogene is expressed in the human cancer cell lines (Clontech), for example HL-60, HeLa, K-562, MOLT-4, Raji, SW480, A549 and G361. As shown in FIG. 20, it was revealed that a 2.4-kb transcript of the HCCRBP2 mRNA was very highly expressed in the promyelocyte leukemia cell line HL-60, the HeLa uterine cancer cell line, the chronic myelogenous leukemia cell line K-562, the lymphoblastic leukaemia cell line MOLT-4, the Burkitt lymphoma cell line Raji, the colon cancer cell line SW480, the lung cancer cell line A549 and the skin cancer melanoma cell line G361.

In order to determine an expression level of the HCCRBP2 gene in uterine cancer, colon cancer and leukemia, the total RNA samples were separated from the normal tissues such as uterus, large intestines and leukocyte obtained from normal healthy humans; and cancer tissues such as uterine cancer, colon cancer and leukemia obtained from patients suffering from uterine cancer, colon cancer and leukemia, using the system RNeasy total RNA kit (Qiagen Inc.). 20 μg of each of the total RNA samples extracted from each of the cancer tissues was electrophoresized in an 1% formaldehyde agarose gel, and then the resultant agarose gel was transferred to a nylon membrane ((Boehringer-Mannheim, Germany). The blots were then hybridized with the ³²P-labeled and randomly primed full-length HCCRBP2 cDNA probes prepared using the Rediprime II random prime labelling system ((Amersham). The northern blotting analysis was repeated twice, and therefore the resultant blots were quantitified with the densitometer and normalized with the β-actin.

FIG. 47(a) shows a northern blotting result to determine whether or not the HCCRBP2 protooncogene is expressed in the normal exocervical tissue, the cervical cancer tissue, the metastatic cervical lymph node tissue and the cervical cancer cell lines (CaSki and CUMC-6). As shown in FIG. 47(a), it was revealed that the expression level of the HCCRBP2 protooncogene was increased in the cervical cancer tissue and the cervical cancer cell lines CaSki and CUMC-6, that is, dominant HCCRBP2 mRNA transcript of approximately 2.4 kb was overexpressed, and the HCCRBP2 protooncogene was the most highly expressed especially in the metastatic cervical lymph node tissue, but very low expressed in the normal tissue. In FIGS. 47(a) and (b), Lane “Normal” represents the normal exocervical tissue, Lane “Cancer” represents the cervical cancer tissue, Lane “metastasis” represents the metastatic cervical lymph node tissue, and each of Lanes “CaSki” and “CUMC-6” represents the uterine cancer cell line. FIG. 47(b) shows the northern blotting result indicating presence of mRNA transcript by hybridizing the same sample with β-actin probe.

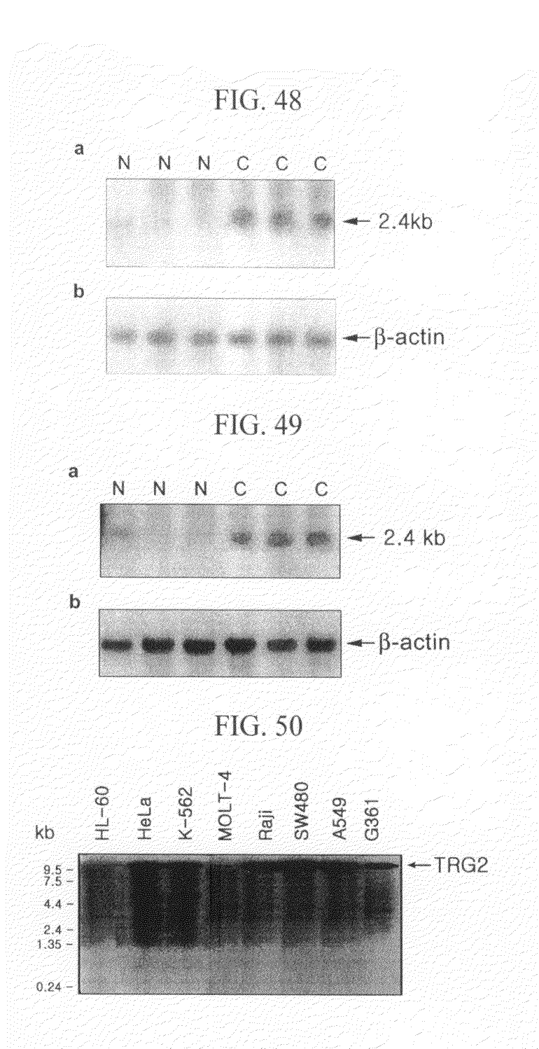

FIG. 48(a) shows a northern blotting result to determine whether or not the HCCRBP2 protooncogene is expressed in the normal large intestine tissues and the colon cancer tissues. FIG. 48(b) shows the northern blotting result indicating presence of mRNA transcript by hybridizing the same sample with β-actin probe. As shown in FIG. 48(a), it was revealed that the 2.4-kb HCCRBP2 mRNA transcript was very highly expressed in the colon cancer tissues, but very slightly expressed in the normal large intestine tissues. In FIGS. 48(a) and (b), Lane “Normal (N)” represents the normal large intestine tissue, and Lane “Cancer (C)” represents the colon cancer tissue.

FIG. 49(a) shows a northern blotting result to determine whether or not the HCCRBP2 protooncogene is expressed in the normal leukocyte tissues and the leukemia tissues. FIG. 49(b) shows the northern blotting result indicating presence of mRNA transcript by hybridizing the same sample with β-actin probe. As shown in FIG. 49(a), it was revealed that the 2.4-kb HCCRBP2 mRNA transcript was very highly expressed in the leukemia tissues, but slightly expressed in the normal leukocyte tissues. In FIGS. 49(a) and (b), Lane “Normal (N)” represents the normal large intestine tissue, and Lane “Cancer (C)” represents the colon cancer tissue.

### EXAMPLE 8

### Size Determination of Protein Expressed after Transforming E. coli with Protooncogene

8-1: PIG5, PIG6, PIG7, PIG11, PIG16, PIG17, PIG19, PIG20, PIG21 and TRG2

Each of the full-length PIG protooncogenes such as PIG5 of SEQ ID NO: 1; PIG6 of SEQ ID NO: 5; PIG7 of SEQ ID NO: 9; PIG11 of SEQ ID NO: 13; PIG16 of SEQ ID NO: 17; PIG17 of SEQ ID NO: 21; PIG19 of SEQ ID NO: 25; PIG20 of SEQ ID NO: 29; PIG21 of SEQ ID NO: 33; and TRG2 of SEQ ID NO: 39 was inserted into a multi-cloning site of the pBAD/thio-Topo vector (Invitrogen, U.S.), and then E. coli Top10 (Invitrogen, U.S.) was transformed with each of the resultant pBAD/thio-Topo/MIG vectors. Each of the expression proteins HT-Thioredoxin is inserted into an upstream region of the multi-cloning site of the pBAD/thio-Topo vector. Each of the transformed E. coli strains was c incubated in LB broth while shaking, and then each of the resultant cultures was diluted at a ratio of 1/100 and incubated for 3 hours. 0.5 mM L-arabinose (Sigma) was added thereto to facilitate production of proteins.

The E. coli cells was sonicated in the culture solutions before/after the L-arabinose induction, and then the sonicated homogenates were subject to 12% sodium dodecyl sulfate-polyacrylamide gel electrophoresis (SDS-PAGE).

FIG. 52 shows a SDS-PAGE result to determine an expression pattern of the proteins in the E. coli Top10 strain transformed with the pBAD/thio-Topo/PIG5 vector, wherein a band of a fusion protein having a molecular weight of approximately 42 kDa was clearly observed after L-arabinose induction. The 42-kDa fusion protein includes the HT-thioredoxin protein having a molecular weight of approximately 15 kDa and the PIG5 protein having a molecular weight of approximately 27 kDa, each protein inserted into the pBAD/thio-Topo/PIG5 vector.

FIG. 53 shows a SDS-PAGE result to determine an expression pattern of the proteins in the E. coli Top10 strain transformed with the pBAD/thio-Topo/PIG6 vector, wherein a band of a fusion protein having a molecular weight of approximately 87 kDa was clearly observed after L-arabinose induction. The 87-kDa fusion protein includes the HT-thioredoxin protein having a molecular weight of approximately 15 kDa and the PIG6 protein having a molecular weight of approximately 72 kDa, each protein inserted into the pBAD/thio-Topo/PIG6 vector.

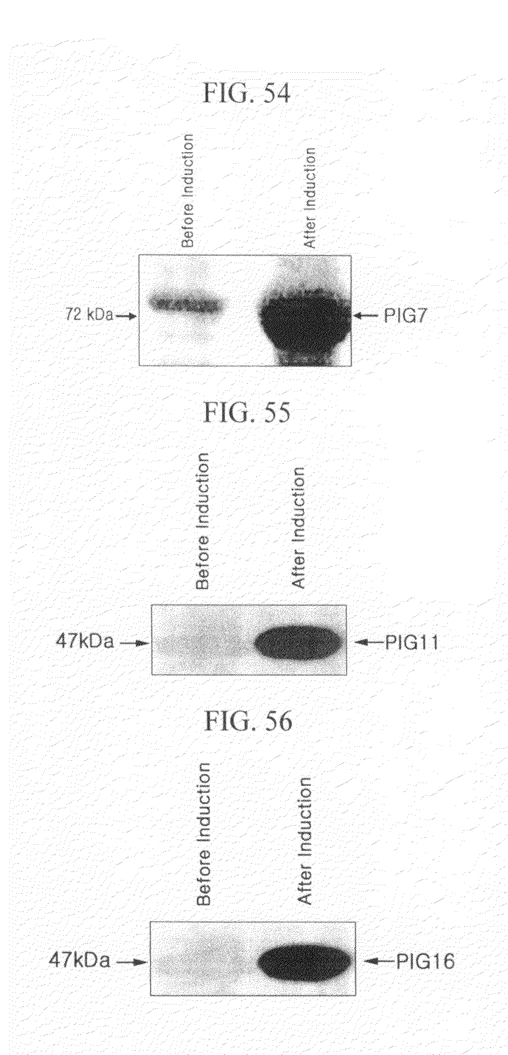

FIG. 54 shows a SDS-PAGE result to determine an expression pattern of the proteins in the E. coli Top10 strain transformed with the pBAD/thio-Topo/PIG7 vector, wherein a band of a fusion protein having a molecular weight of approximately 24 kDa was clearly observed after L-arabinose induction. The 24-kDa fusion protein includes the HT-thioredoxin protein having a molecular weight of approximately 15 kDa and the PIG7 protein having a molecular weight of approximately 9 kDa, each protein inserted into the pBAD/thio-Topo/PIG7 vector.

FIG. 55 shows a SDS-PAGE result to determine an expression pattern of the proteins in the E. coli Top10 strain transformed with the pBAD/thio-Topo/PIG11 vector, wherein a band of a fusion protein having a molecular weight of approximately 47 kDa was clearly observed after L-arabinose induction. The 47-kDa fusion protein includes the HT-thioredoxin protein having a molecular weight of approximately 15 kDa and the PIG11 protein having a molecular weight of approximately 32 kDa, each protein inserted into the pBAD/thio-Topo/PIG11 vector.

FIG. 56 shows a SDS-PAGE result to determine an expression pattern of the proteins in the E. coli Top10 strain transformed with the pBAD/thio-Topo/PIG16 vector, wherein a band of a fusion protein having a molecular weight of approximately 47 kDa was clearly observed after L-arabinose induction. The 47-kDa fusion protein includes the HT-thioredoxin protein having a molecular weight of approximately 15 kDa and the PIG16 protein having a molecular weight of approximately 32 kDa, each protein inserted into the pBAD/thio-Topo/PIG16 vector.

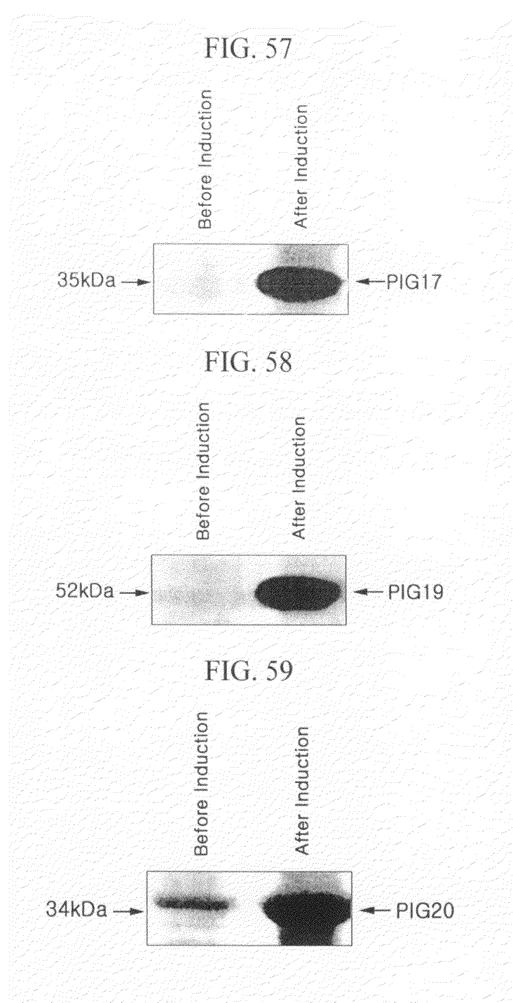

FIG. 57 shows a SDS-PAGE result to determine an expression pattern of the proteins in the E. coli Top10 strain transformed with the pBAD/thio-Topo/PIG17 vector, wherein a band of a fusion protein having a molecular weight of approximately 35 kDa was clearly observed after L-arabinose induction. The 35-kDa fusion protein includes the HT-thioredoxin protein having a molecular weight of approximately 15 kDa and the PIG17 protein having a molecular weight of approximately 20 kDa, each protein inserted into the pBAD/thio-Topo/PIG17 vector.

FIG. 58 shows a SDS-PAGE result to determine an expression pattern of the proteins in the E. coli Top 10 strain transformed with the pBAD/thio-Topo/PIG19 vector, wherein a band of a fusion protein having a molecular weight of approximately 52 kDa was clearly observed after L-arabinose induction. The 52-kDa fusion protein includes the HT-thioredoxin protein having a molecular weight of approximately 15 kDa and the PIG19 protein having a molecular weight of approximately 37 kDa, each protein inserted into the pBAD/thio-Topo/PIG19 vector.

FIG. 59 shows a SDS-PAGE result to determine an expression pattern of the proteins in the E. coli Top10 strain transformed with the pBAD/thio-Topo/PIG20 vector, wherein a band of a fusion protein having a molecular weight of approximately 34 kDa was clearly observed after L-arabinose induction. The 34-kDa fusion protein includes the HT-thioredoxin protein having a molecular weight of approximately 15 kDa and the PIG20 protein having a molecular weight of approximately 19 kDa, each protein inserted into the pBAD/thio-Topo/PIG20 vector.

FIG. 60 shows a SDS-PAGE result to determine an expression pattern of the proteins in the E. coli Top10 strain transformed with the pBAD/thio-Topo/PIG21 vector, wherein a band of a fusion protein having a molecular weight of approximately 45 kDa was clearly observed after L-arabinose induction. The 45-kDa fusion protein includes the HT-thioredoxin protein having a molecular weight of approximately 15 kDa and the PIG21 protein having a molecular weight of approximately 30 kDa, each protein inserted into the pBAD/thio-Topo/PIG21 vector.

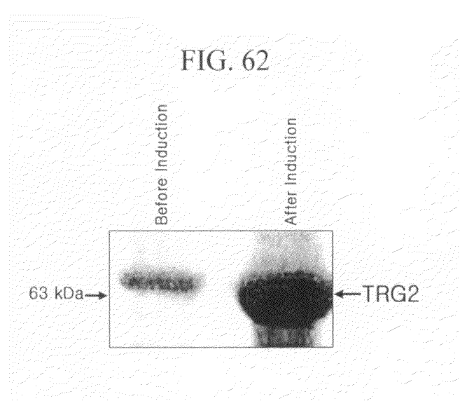

FIG. 62 shows a SDS-PAGE result to determine an expression pattern of the proteins in the E. coli Top 10 strain transformed with the pBAD/thio-Topo/TRG2 vector, wherein a band of a fusion protein having a molecular weight of approximately 63 kDa was clearly observed after L-arabinose induction. The 63-kDa fusion protein includes the HT-thioredoxin protein having a molecular weight of approximately 15 kDa and the TRG2 protein having a molecular weight of approximately 48 kDa, each protein inserted into the pBAD/thio-Topo/TRG2 vector.

8-2: HCCRBP2

A full-length coding region of the HCCRBP2 protooncogene (SEQ ID NO: 37), which corresponds to nucleotide sequence positions from 9 to 356 and is expected to encode a protein having amino acid numbers from 1 to 115 of SEQ ID NO: 38, was inserted between restriction enzymes BamHI and NotI in a multi-cloning site of a GST-fused pGEX 4T-3 vector (Amersham Pharmacia Biotech) to obtain an expression vector pGEX4T-3/HCCRBP2, and then E. coli BL21 (ATCC 47092) was transformed with the resultant pGEX4T-3/HCCRBP2 vector. The transformed E. coli strain was incubated at 37° C. in a LB culture solution for 16 hours while shaking, and then the resultant culture solution was diluted at a ratio of 1/100 and incubated for 3 hours again. 1 mM isopropyl beta-D-thiogalacto-pyranoside (IPTG, Sigma) was added thereto to facilitate production of proteins.

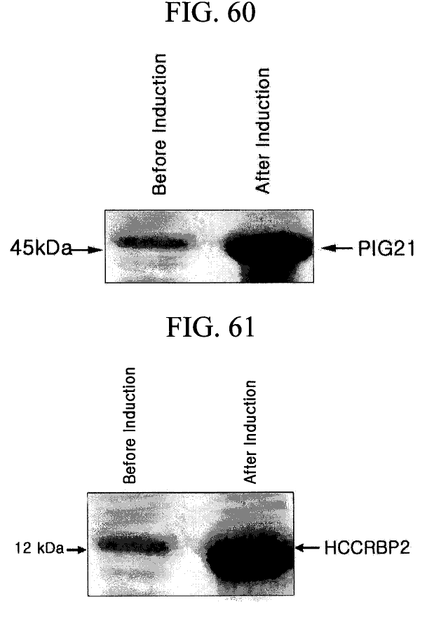

The E. coli cells was sonicated in samples of the culture solution before/after the L-arabinose induction, and then the sonicated homogenates were subject to 12% sodium dodecyl sulfate-polyacrylamide gel electrophoresis (SDS-PAGE). FIG. 61 shows a SDS-PAGE result to determine an expression pattern of the proteins in the E. coli BL21 strain transformed with the pGEX4T-3/HCCRBP2 vector, wherein a band of a fusion protein having a molecular weight of approximately 38 kDa was clearly observed after IPTG induction. The 38-kDa fusion protein includes the GST protein having a molecular weight of approximately 26 kDa expressed from the pGEX4T-3 vector.

### INDUSTRIAL APPLICABILITY

The protooncogene of the present invention is a novel gene, and may be effectively used for diagnosing the cancers, including leukemia, uterine cancer, lymphoma, colon cancer, lung cancer, skin cancer, etc., as well as producing transformed animals, etc.

1. A human protooncoprotein having an amino acid sequence selected from the group consisting of SEQ ID NO: 2; SEQ ID NO: 6; SEQ ID NO: 10; SEQ ID NO: 14; SEQ ID NO: 18; SEQ ID NO: 22; SEQ ID NO: 26; SEQ ID NO: 30; SEQ ID NO: 34; SEQ ID NO: 38; and SEQ ID NO: 40.

2. A human protooncogene having a DNA sequence selected from the group consisting of a DNA sequence corresponding to nucleotide sequence positions from 9 to 746 of SEQ ID NO: 1; a DNA sequence corresponding to nucleotide sequence positions from 293 to 2302 of SEQ ID NO: 5; a DNA sequence corresponding to nucleotide sequence positions from 3536 to 3792 of SEQ ID NO: 9; a DNA sequence corresponding to nucleotide sequence positions from 50 to 931 of SEQ ID NO: 13; a DNA sequence corresponding to nucleotide sequence positions from 696 to 1577 of SEQ ID NO: 17; a DNA sequence corresponding to nucleotide sequence positions from 59 to 610 of SEQ ID NO: 21; a DNA sequence corresponding to nucleotide sequence positions from 32 to 1030 of SEQ ID NO: 25; a DNA sequence corresponding to nucleotide sequence positions from 1 to 498 of SEQ ID NO: 29; a DNA sequence corresponding to nucleotide sequence positions from 146 to 961 of SEQ ID NO: 33; a DNA sequence corresponding to nucleotide sequence positions from 9 to 356 of SEQ ID NO: 37; and a DNA sequence corresponding to nucleotide sequence positions from 747 to 2066 of SEQ ID NO: 39, wherein each of the DNA sequences encodes the protooncoprotein as defined in claim 1.

3. The human protooncogene according to claim 2, wherein the protooncogene has a DNA sequence selected from the group consisting of SEQ ID NO: 1; SEQ ID NO: 5; SEQ ID NO: 9; SEQ ID NO: 13; SEQ ID NO: 17; SEQ ID NO: 21; SEQ ID NO: 25; SEQ ID NO: 29, SEQ ID NO: 33, SEQ ID NO: 37 and SEQ ID NO: 39.

4. A vector comprising each of the protooncogenes as defined in claim 2.

5. A kit for diagnosing cancer and cancer metastasis including each of the protooncoproteins as defined in claim 1.

6. A kit for diagnosing cancer and cancer metastasis including each of the protooncogenes as defined in claim 2.

7. A vector comprising each of the protooncogenes as defined in claim 3.

8. A kit for diagnosing cancer and cancer metastasis including each of the protooncogenes as defined in claim 3


SEQUENCE LISTING (42 sequences)

SEQ ID NO: 1 - DNA, 1009 bp, Homo sapiens
gcttcaagatggcggtgcaggagtcggcggctcagttgtccatgaccctgaaggtccaggagtacccgaccctcaaggtgccctacgagacgctgaacaaatgctttcgcgccgctcagaagaacattgaccgggagaccagccacgtcaccatggtggtggccgagctggagaagacgttgagcggctgccccgccgtggactccgtggtcagcctgctggacggcgtggtggagaagctcagcgtcctcaagaggaaggcggtggaatccatccaggccgaggacgagagcgccaagctgtgcaagcgccggatcgagcacctcaaagagcatagcagcgaccagcccgcggcggccagcgtgtggaagaggaagcgcatggatcgcatgatggtggagcacctgctgcgttgcggctactacaacacggctgtcaagctggcgcgccagagcggcatcgagacatgcaagaaagcacttcagccaagcagaagggagccagctggacgaggtgcgccaggccatgggcatgctggccttcccgcccgacacgcacatctccccgtacaaggaccttctggaccctgcacggtggcggatgctgatccagcagttccggtacgacaactaccgactacaccagctgggaaacaattctgtgttcaccctcaccctgcaggccggcctctcagccatcaagacaccacagtgctacaaggaggacggcagctccaagagccctgactgccctgtgtgcagccgctccctgaacaagctggcgcagcccctgctcatggcccactgtgccaactcccgcctggtctgcaagatttctggcgacgtgatgaacgagaacaatccgcccatgatgctgcccaacggctacgtctacggctacaattctctgctttctatccgtcaagatgataaagtcgtgtgcccgagaaccaaagaagtcttccacttctcacaagccgagaaggtgtacatcatgtaggccccacgtc

SEQ ID NO: 2 - protein, 245 aa, Homo sapiens [sequence omitted]

SEQ ID NO: 3 - DNA, 16 bp, Artificial Sequence
  H-T11G primer
aagctttttttttttg

SEQ ID NO: 4 - DNA, 13 bp, Artificial Sequence
  H-AP9 primer
aagcttcattccg

SEQ ID NO: 5 - DNA, 2964 bp, Homo sapiens
gaagcctgaaccacctgttaatctgaagtacaatgcacccacgtctcatgttactccgtccgtcaagaaaagaagcagcaccttatctcagctccctggggataagtccaaagcctttgatttccttagtgaagaaactgaagctagtttagcctcacgcagagaacaaaagagagagcagtatcgtcaggtaaaagcacatgttcagaaggaagacggtagagtgcaggcttttggctggagtctgcctcagaagtacaaacaggtaaccaatggtcaaggtgaaaataagatgaaaaatttacctgtgcctgtctatctcagacctctggatgaaaaagatacatcaatgaagctgtggtgtgctgttggagtcaatttatctggtgggaagaccagagatggtggttctgttgttggagcaagtgtattttacaaggatgttgctggtttggatacagaaggcagtaaacagcgaagtgcctctcagagtagtttagataagttagatcaggaacttaaggaacagcagaaggagttaaaaaatcaagaagaattatccagtctagtttggatctgtaccagcactcattcggctacaaaagttcttattattgatgctgttcaacctggcaacatcctagacagtttcactgtttgcaactctcatgttctgtgcattgcaagtgtgccaggtgcacgagaaacagactaccctgcaggagaagatctttcagaatctggtcaggtagacaaagcatctttatgtggaagtatgacaagcaacagctcagcagagacagacagcctgttaggaggcatcacagtggttggttgttctgcagaaggtgtgacgggagctgccacttcccctagtacaaatggtgcttctccagtgatggataaaccaccagaaatggaagcagaaaatagtgaggttgatgaaaatgttccaacagcagaagaagcaactgaagctacagaagggaatgcggggtcagctgaagacacagtggacatctcccaaactggcgtctacacagagcatgtctttacagatcctttgggagttcagatcccagaagacctctccccagtgtatcagtcgagcaatgactcagatgcatataaagatcaaatatcagtactgccaaatgaacaagacttggtgagagaagaagcccagaaaatgagtagtcttttaccaactatgtggcttggagctcaaaatggctgtttgtatgtccattcatctgtagcccagtggaggaaatgtctccattccattaaacttaaagattcgattctcagtattgtacacgtgaagggaatcgtgttagtagccctggctgacggcacccttgcaatctttcacagaggagtggatgggcagtgggatttgtcaaactatcacctcttagaccttggacggcctcatcattccatccgttgcatgactgtggtacatgacaaagtctggtgtggctataggaacaaaatctatgtggtgcagccaaaggccatgaaaatagagaaatcttttgatgcacatcccaggaaggagagccaagtgcgacagcttgcgtgggtgggggatggcgtgtgggtctccattcgcttggattctacgctccgtctctatcatgcacacacttatcaacatctacaggatgtggacattgagccttatgtaagcaaaatgttaggtactggaaaactgggcttctcttttgtgagaattacagctcttatggtgtcttgtaatcgtttgtgggtggggacaggaaatggtgtcattatctccatcccattgacagaaaccgtaatcctccaccagggacgtttactggggctgagggcaaataaaacctcaggtgtaccaggaaatcgtcctggaagtgtaatccgtgtatatggtgatgaaaacagtgataaagtgactccagggacatttataccctattgttcaatggcacatgcacagctttgcttccatgggcaccgggatgctgtgaaattctttgtggcagtcccaggtcaagtcatcagcccacaaagtagcagtagtggcacggatctgacgggtgacaaagcagggccatctgcacaggagcctggtagtcagacgcccttgaagtctatgcttgtcatcagtggaggagagggctacatcgacttccgaatgggtgatgaaggtggagaatcagaacttcttggagaggatcttccacttgaaccttctgtcaccaaagcagaaaggagtcacttgatagtgtggcaagtgatgtatggcaatgagtgagcccatgggaaacaggtggagatggggaagccgtctcttctgcatggtttattttccctctatccttttatttaatgctcttttgtgagataagtttcaccacataatgtgtgagcattttttcctgttaactttatattacaaaatccgttctaccataacaatacagaggaactagctgtgttactgcaccagtgttataggtaacttcagtatattatgaacaaatcaaagaatgtttacttcctgcaaactggtgaattatagaaagcaatccagatgtggtttactctgccacagtctaatgtcattcacttcatttgatggggtcacttgttagctgtcactaataatggaattaatgggaaacacttgataaatgaaactgtaccgttaaatacaatagctgagttttccccagtgtattgtaaaattgacacacactaatacaagatggattatcaggatgtatctaaatgtcccgagagggttaaaagctaactgtaaattactttaactttcactttcaaatctgacaaatcttgtttatatggtatataaaactttgttttcatcagatttgggggggttttatattaaagaaattaaccctaaaaaaaaaaaaaaaaaaaaaaaaaaaaaaaaaaaaaaaaaaaaaaaaa

SEQ ID NO: 6 - protein, 669 aa, Homo sapiens
MKNLPVPVYLRPLDEKDTSMKLWCAVGVNLSGGKTRDGGSVVGASVFYKDVAGLDTEGSKQRSASQSSLDKLDQELKEQQKELKNQEELSSLVWICTSTHSATKVLIIDAVQPGNILDSFTVCNSHVLCIASVPGARETDYPAGEDLSESGQVDKASLCGSMTSNSSAETDSLLGGITVVGCSAEGVTGAATSPSTNGASPVMDKPPEMEAENSEVDENVPTAEEATEATEGNAGSAEDTVDISQTGVYTEHVFTDPLGVQIPEDLSPVYQSSNDSDAYKDQISVLPNEQDLVREEAQKMSSLLPTMWLGAQNGCLYVHSSVAQWRKCLHSIKLKDSILSIVHVKGIVLVALADGTLAIFHRGVDGQWDLSNYHLLDLGRPHHSIRCMTVVHDKVWCGYRNKIYVVQPKAMKIEKSFDAHPRKESQVRQLAWVGDGVWVSIRLDSTLRLYHAHTYQHLQDVDIEPYVSKMLGTGKLGFSFVRITALMVSCNRLWVGTGNGVIISIPLTETVILHQGRLLGLRANKTSGVPGNRPGSVIRVYGDENSDKVTPGTFIPYCSMAHAQLCFHGHRDAVKFFVAVPGQVISPQSSSSGTDLTGDKAGPSAQEPGSQTPLKSMLVISGGEGYIDFRMGDEGGESELLGEDLPLEPSVTKAERSHLIVWQVMYGNE

SEQ ID NO: 7 - DNA, 16 bp, Artificial Sequence
  H-T11A primer
aagcttttttttttta

SEQ ID NO: 8 - DNA, 13 bp, Artificial Sequence
  H-AP32 primer
aagcttcctgcaa

SEQ ID NO: 9 - DNA, 4301 bp, Homo sapiens
cggcggaattgtcgacggccattaccaatcgcgaaaccaattataacactagggaaaaatttgtagcggatggcagtgttgaaggcaaatgtaaacataagggtaatggtctgtcatggcttttagaaaaagatgatagagttcatcattattttgccttcatctttgttaaggacagaaaattccctgacaggtgggcaagtatcaggttacctattttttattcctttggtacaaaagggttgaacgtcaggctaaaaaagcagccatgcatttattattaagcattttctaccgacaaggcactgtgctaggtactgtaatcctaccataagtaggtaggtatttcttccactgtaaatcataggggtttgctgttttatgtgagttagcctcttccccttgtctgagcattcctcaggggaggtcacctgtgaggttcccagaactgtagttttttttaccagggtgttgtatttggagggggaggaggactcggctcaaaagagctagctggctctccagtgttcagaggtgagtccacgatactcttaccacaatttggaagtttgtgaatctttttaaagaactaatcaatctctaatagcattgaggttgtacctacatattaagttgaatggactgttctatttaaaaaataaacaactagacaattaactagtttattaacctatcacaattgaatttttttttaattttcagtcttaacacattttttaaaatgtattaaagtaatacattgtagtagtaggattatatactccttggctgagaattccaagtactgtggttctactgtttagtggaaaactctggaagttaaaatatagaatatgagaggaggcttttttataatgggcatcattgtgtggaaaatgacccatgtgaatacaaatatttcctagttcagagattttggttatatctggtgcttggatcaagtttaaaaatggaaggtgagattttgcatgagcctattaaaaagcatagtaataaatgcaaggccagctggtggaaaagtgaggcagaatggagcttgtttataggttttctgataacaattataaaaaatgtgctttatagattaagatttattgaagtataaatatgtagtaatgatataatgtattttaagttatacaagaaaatgtagggacttttgtttgggtctttttctctttgtggctgaggggaaacaagtcagtgtccaataaagctgtaaactcctctgctctaagataaatgatgtgatttatttatttataactggcttctttccaagtaggttttcaggtggcatatttggaagacggctggaatgacagaattcttgtatcagagtaggtaagaagggagcaacctctcaatggctattatgtcatgctttttaagatcgtatgcggttcctatatacaaggaagcttccctgtggtagatttgtcataaatgccaaagatatttggtaatgtgagtgtagaaaaagtagtatgagggttggcaaatactgtttttgtcttggcagctctaatatctgcattgttcagaaaggattctgaggctaaggcaaagctctgtgggaaaaagggactggaccaaaaaaaactggatggtggcacacaagaagaggaaatgatgagatgtgtactttctatctctggttaggcttagtccccactagacaaattgattttaaattccatccacacattcattacccacacagataatcatagaaatttgggggagtgcctagcttctgatgaagtggtgatatggcagttgccaagcagtggcatcgccagagtatctgtttggttagcaaatgagcagtcattttaggtcatgcagattgctgatatctgcccagtagccactgagcatttgctggttttttcttctggctttcttggaggttaagctctctgtagtcatacccagttggtacttgatctttagcaatatgtctcatattcatgtaaattgaagggagggttacatgtactgaaataatctgcatgctaggcattggcttagacaccgtacctatctcacttagatttgtggactaggaaagcaagattcagagatcatgtgacttgcatgtggcctagagataaaattcaaatctggttctgtagactccagggacatattcaccatgccatgggtggtggctattaaaccttgataaatttgtgtttatggttaacaaatgtgaaagctattaaacattgctggtttgaattttttacagtgcagaaatgtaaaatgaaaaaggatatttcctttcacagtgttaccgagaagtcatgataatttcgtttgttcttccagatttaggcatatacttatttaatcaataatgtgttaacagctgacacctgtggttgctgtgacaggcactatttgaagtgctttatcatggattaactcttaatcctcagctaccgtatacagtaggacataaccccatttcacatgcactacactgagacttgcctcctctccccccacattgaagatgttcttttttcataactatatactattccattgcatgaatattctgtaatttatttaatcccctatggattgataattaggttcattatagatagaagtgtaattaacattcctgtacatgtattttgctacttgtgtgggtatttctgtaggatgaataactagaaatttattggatcaggtttcacatttgcagttttgaaaactactaccaaaaagatttcaccaatttacaactccatcattagtaagaatgcctgtttgcctatagtctgccaaccctgaatccttaaaaatttttgccaatctggtaggcaaaatttctttcttttctttgaatattaatgaggaggaacatcttttcatgtttcttggccatttgcatttcctattatgaattgcttttgcccattttcctttttttaattatgaaagtctaatgactaccttctcattgtataaaaaacacagttctttgaatagagagacccttttctccaatgctaccaatcacattccacttaccacagtttaacatacatcctctagtcacctttccgtacgaatatacatacacataaaaacactttttacataaataggatctcatattctgtagctttttaaaattttggtctcaaaaaaagataacaggtctttaaatttctttaatggttgaatatgattaaatactatgaaaatgccattatttattcccttaatttttttcctctcgctattacattgccaaagtaaacatcctattcagatgtctttgtgcatgtgtgtgaatatttctttagtctggagtccagtaaggtggatttttggatcaaagggtttgttctctgtccaccttcagtcttcccaaaggccttcataactgtattttcaccaagtgtatggagaatgttcatttccccatataaccatacctacacttgatagtttttatctgttgggcgaaaaagaaccttttcttattttgcatttccctgattataaaaaaaaatggtgagattggggttattttcatgtttattggccatttatagtttactgtggattgtttgtatcccttacctgctttctattgggttatgtgtggatatattgtttttatttgttcagcatctccttccccatcttctggtaacacaacctttatttatttgtggggaacctattccctgtggcttaggtgagcatgtgaccaggcctggcctcctgagtcccacagcttcctagccacagtgataaaagaatgggtatataacttaagccaggctaaggaaagcccttaacagaacttctgctggaactactggaaagaaggctttatggagatcccaggaaccaaggaccatgtaagcctgaatttgtgccatgtggagagagtctgtctgaggagaaactcggatgctagcagaaatggaaagagaactaagttctgatgtcatttttctggaggccctagatccagctgtgcctaaagcctgccctacctccggactttaaagttttgtgagccaataaagtccctttcttgtttaagataattgaattgagtttctgttctgattaatataggttatttgtattttcttattgatttgtagaaaacctttgtaattttaaattctagactttatgcactatataagttaataaaattagcatggccttccatgaaaaaaaaaaaaaaaaaaaaaaaaaaaaaaaaaaaaaaaaaaaaaaaaaaaaaaaaaaaaaaaaaa

SEQ ID NO: 10 - protein, 78 aa, Homo sapiens [sequence omitted]

SEQ ID NO: 11 - DNA, 16 bp, Artificial Sequence
  H-T11A primer
aagcttttttttttta

SEQ ID NO: 12 - DNA, 13 bp, Artificial Sequence
  H-AP27 primer
aagcttctgctgg

SEQ ID NO: 13 - DNA, 1038 bp, Homo sapiens
gctccgcgccggggtccgagtcccacgaagccccggcccgagccgccggatgcccgcgcgcagcggggcccagttttgccgacggatggggcaaaagaagcagcgaccagctagagcagggcagccacacagctcgtccgacgcagcccaggcacctgcagagcagccacacagctcgtccgatgcagcccaggcaccttgccccagggagcgctgcttgggaccgcccaccactccgggcccataccgcagcatctatttctcaagcccaaagggccaccttacccgactggggttggagttcttcgaccagccggcagtccccctggcccgggcatttctgggacaggtcctagtccggcgacttcctaatggcacagaactccgaggccgcatcgtggagaccgaggcatacctggggccagaggatgaagccgcccactcaaggggtggccggcagaccccccgcaaccgaggcatgttcatgaagccggggaccctgtacgtgtacatcatttacggcatgtacttctgcatgaacatctccagccagggggacggggcttgcgtcttgctgcgagcactggagcccctggaaggtctggagaccatgcgtcagcttcgcagcaccctccggaaaggcaccgccagccgtgtcctcaaggaccgcgagctctgcagtggcccctccaagctgtgccaggccctggccatcaacaagagctttgaccagagggacctggcacaggatgaagctgtatggctggagcgtggtcccctggagcccagtgagccggctgtagtggcagcagcccgggtgggcgtcggccatgcaggggagtgggcccggaaacccctccgcttctatgtccggggcagcccctgggtcagtgtggtcgacagagtggctgagcaggacacacaggcctgagcaaagggcctgcccagacaagattttttaattgtttaaaaaccgaataaatgttttatttctagaaaaaaaaaaaaaaaaaaaaaaaaaaaaaaaaacaaaaaaaa

SEQ ID NO: 14 - protein, 293 aa, Homo sapiens
MPARSGAQFCRRMGQKKQRPARAGQPHSSSDAAQAPAEQPHSSSDAAQAPCPRERCLGPPTTPGPYRSIYFSSPKGHLTRLGLEFFDQPAVPLARAFLGQVLVRRLPNGTELRGRIVETEAYLGPEDEAAHSRGGRQTPRNRGMFMKPGTLYVYIIYGMYFCMNISSQGDGACVLLRALEPLEGLETMRQLRSTLRKGTASRVLKDRELCSGPSKLCQALAINKSFDQRDLAQDEAVWLERGPLEPSEPAVVAAARVGVGHAGEWARKPLRFYVRGSPWVSVVDRVAEQDTQA

SEQ ID NO: 15 - DNA, 16 bp, Artificial Sequence
  H-T11G primer
aagcttttttttttta

SEQ ID NO: 16 - DNA, 13 bp, Artificial Sequence
  H-AP11 primer
aagcttcgggtaa

SEQ ID NO: 17 - DNA, 1682 bp, Homo sapiens
caagtatctattgcttagcatatgtaaagttgtagtctatatttatggggccattgcttaaagattataaattatgtaaatacattaataaattctaagtttcatttgacattccattgaatctcgcacccagtcttgcgtatgcctgcccagttttcagcctcttaacgggagactcaagcacattggtattgtataaaggtatagagcacttagcttacaatctttaaaggtttctctgccttcccttctacccacccgcctcccaccagatcccatctggaaatcataataaagacatatgccactttgacaaacctgactagtccttactagcctgagggtaaaagattaagctccaacctcaagtcatttacctggtcttggtaataagtttcttttagcttgtacagcatcctcagaccaactgaggagctttccttgttaacaatttagcttatctttctgtttcctttatttttcccctgcctctgttagtggttaacactcttttccctcagggagcctaatgaggtttttaatatcatctaaaaataaagcattgaagtgaagttggtgaaaaaaaaaaaaaaaaaaaaaaaaaaaaaaaaaaaaaaaggccgcctcggccgtcggcggctgctgggctccgcgccggggtccgagtcccacgaagccccggcccgagccgccggatgcccgcgcgcagcggggcccagttttgccgacggatggggcaaaagaagcagcgaccagctagagcagggcagccacacagctcgtccgacgcagcccaggcacctgcagagcagccacacagctcgtccgatgcagcccaggcaccttgccccagggagcgctgcttgggaccgcccaccactccgggcccataccgcagcatctatttctcaagcccaaagggccaccttacccgactggggttggagttcttcgaccagccggcagtccccctggcccgggcatttctgggacaggtcctagtccggcgacttcctaatggcacagaactccgaggccgcatcgtggagaccgaggcatacctggggccagaggatgaagccgcccactcaaggggtggccggcagaccccccgcaaccgaggcatgttcatgaagccggggaccctgtacgtgtacatcatttacggcatgtacttctgcatgaacatctccagccagggggacggggcttgcgtcttgctgcgagcactggagcccctggaaggtctggagaccatgcgtcagcttcgcagcaccctccggaaaggcaccgccagccgtgtcctcaaggaccgcgagctctgcagtggcccctccaagctgtgccaggccctggccatcaacaagagctttgaccagagggacctggcacaggatgaagctgtatggctggagcgtggtcccctggagcccagtgagccggctgtagtggcagcagcccgggtgggcgtcggccatgcaggggagtgggcccggaaacccctccgcttctatgtccggggcagcccctgggtcagtgtggtcgacagagtggctgagcaggacacacaggcctgagcaaagggcctgcccagacaagattttttaattgtttaaaaaccgaataaatgttttatttctagaaaaaaaaaaaaaaaaaaaaaaaacaaaaaaaaaaaaaaa

SEQ ID NO: 18 - protein, 293 aa, Homo sapiens; identical to SEQ ID NO: 14

SEQ ID NO: 19 - DNA, 16 bp, Artificial Sequence
  H-T11C primer
aagctttttttttttc

SEQ ID NO: 20 - DNA, 13 bp, Artificial Sequence
  H-AP16 primer
aagctttagagcg

SEQ ID NO: 21 - DNA, 626 bp, Homo sapiens
tgttgtggagtacgctttggactgagaagcatcgaggctataggacgcagctgttgccatgacggcccaggggggcctggtggctaaccgaggccggcgcttcaagtgggccattgagctaagcgggcctggaggaggcagcaggggtcgaagtgaccggggcagtggccagggagactcgctctacccagtcggtcacttggacaagcaagtgcctgataccagcgtgcaagagacagaccggatcctggtggagaagcgctgctgggacatcgccttgggtcccctcaaacagattcccatgaatctcttcatcatgtacatggcaggcaatactatctccatcttccctactatgatggtgtgtatgatggcctggcgacccattcaggcacttatggccatttcagccactttcaagatgttagaaagttcaagccagaggtttcttcagggtttggtctatctcattgggaacctgatgggtttggcattggctgtttacaagtgccagtccatgggactgttacctacacatgcatcggattggttagccttcattgagccccctgagagaatggagttcagtggtggaggactgcttttgtgaacatgagaaagcagcg

SEQ ID NO: 22 - protein, 183 aa, Homo sapiens [sequence omitted]

SEQ ID NO: 23 - DNA, 16 bp, Artificial Sequence
  H-T11C primer
aagctttttttttttc

SEQ ID NO: 24 - DNA, 13 bp, Artificial Sequence
  H-AP18 primer
aagcttagaggca

SEQ ID NO: 25 - DNA, 1031 bp, Homo sapiens
attcccgattccttttggttccaagtccaatatggcaactctaaaggatcagctgatttataatcttctaaaggaagaacagaccccccagaataagattacagttgttggggttggtgctgttggcatggcctgtgccatcagtatcttaatgaaggacttggcagatgaacttgctcttgttgatgtcatcgaagacaaattgaagggagagatgatggatctccaacatggcagccttttccttagaacaccaaagattgtctctggcaaagactataatgtaactgcaaactccaagctggtcattatcacggctggggcacgtcagcaagagggagaaagccgtcttaatttggtccagcgtaacgtgaacatatttaaattcatcattcctaatgttgtaaaatacagcccgaactgcaagttgcttattgtttcaaatccagtggatatcttgacctacgtggcttggaagataagtggttttcccaaaaaccgtgttattggaagtggttgcaatctggattcagcccgattccgttacctgatgggggaaaggctgggagttcacccattaagctgtcatgggtgggtccttggggaacatggagattccagtgtgcctgtatggagtggaatgaatgttgctggtgtctctctgaagactctgcacccagatttagggactgataaagataaggaacagtggaaagaggttcacaagcaggtggttgagagtgcttatgaggtgatcaaactcaaaggctacacatcctgggctattggactctctgtagcagatttggcagagagtataatgaagaatcttaggcgggtgcacccagtttccaccatgattaagggtctttacggaataaaggatgatgtcttccttagtgttccttgcattttgggacagaatggaatctcagaccttgtgaaggtgactctgacttctgaggaagaggcccgtttgaagaagagtgcagatacactttgggggatccaaaaggagctgcaattttaaa

SEQ ID NO: 26 - protein, 332 aa, Homo sapiens
MATLKDQLIYNLLKEEQTPQNKITVVGVGAVGMACAISILMKDLADELALVDVIEDKLKGEMMDLQHGSLFLRTPKIVSGKDYNVTANSKLVIITAGARQQEGESRLNLVQRNVNIFKFIIPNVVKYSPNCKLLIVSNPVDILTYVAWKISGFPKNRVIGSGCNLDSARFRYLMGERLGVHPLSCHGWVLGEHGDSSVPVWSGMNVAGVSLKTLHPDLGTDKDKEQWKEVHKQVVESAYEVIKLKGYTSWAIGLSVADLAESIMKNLRRVHPVSTMIKGLYGIKDDVFLSVPCILGQNGISDLVKVTLTSEEEARLKKSADTLWGIQKELQF

SEQ ID NO: 27 - DNA, 16 bp, Artificial Sequence
  H-T11G primer
aagctttttttttttg

SEQ ID NO: 28 - DNA, 13 bp, Artificial Sequence
  H-AP16 primer
aagctttagagcg

SEQ ID NO: 29 - DNA, 526 bp, Homo sapiens
atgctgccacgtgatgagagaagattcaaagctgcagacctcaatggtgacctgacagctactcgggaggagttcactgcctttctgcatcctgaagagtttgaacatatgaaggaaattgtggttttggaaaccctggaggacatcgacaagaacggggatgggttcgtggatcaggatgagtatattgcggatatgttttcccatgaggagaatggccctgagccagactgggttttatcagaacgggagcagtttaacgaattccgggatctgaacaaggacgggaagttagacaaagatgagattcgccactggatcctccctcaagattatgatcacgcacaggctgaggccaggcatctggtatatgaatcagacaaaaacaaggatgagaagctaactaaagaggaaatattggagaactggaacatgtttgttggaagccaagctaccaattacggggaagatctcacaaaaaatcatgatgagctttgatagacactcaccagaatatggcagactg

SEQ ID NO: 30 - protein, 165 aa, Homo sapiens
MLPRDERRFKAADLNGDLTATREEFTAFLHPEEFEHMKEIVVLETLEDIDKNGDGFVDQDEYIADMFSHEENGPEPDWVLSEREQFNEFRDLNKDGKLDKDEIRHWILPQDYDHAQAEARHLVYESDKNKDEKLTKEEILENWNMFVGSQATNYGEDLTKNHDEL

SEQ ID NO: 31 - DNA, 16 bp, Artificial Sequence
  H-T11G primer
aagctttttttttttg

SEQ ID NO: 32 - DNA, 13 bp, Artificial Sequence
  H-AP17 primer
aagcttaccaggt

SEQ ID NO: 33 - DNA, 965 bp, Homo sapiens
gccatgactgagcagatgacccttcgtggcaccctcaagggccacaacggctgggtaacccagatcgctactaccccgcagttcccggacatgatcctctccgcctctcgtgataagaccatcatcatgtggaaactgaccagggatgagaccaactatggaattccacagcgtgctctgcggggtcactcccactttgttagtgatgtggttatctcctcagatggccagtttgccctctcaggctcctgggatggaaccctgcgcctctgggatctcacaacgcaagggcaccaccacgaggcgatttgtgggccataccaaggatgtgctgagtgtggccttctcctctgacaaccggcagattgtctctggatctcgagataaaaccatcaagctatggaataccctgggtgtgtgcaaatacactgtccaggatgagagccactcagagtgggtgtcttgtgtccgcttctcgcccaacagcagcaaccctatcatcgtctcctgtggctgggacaagctggtcaaggtatggaacctggctaactgcaagctgaagaccaaccacattggccacacaggctatctgaacacggtgactgtctctccagatggatccctctgtgcttctggaggcaaggatggccaggccatgttatgggatctcaacgaaggcaaacacctttacacgctagatggtggggacatcatcaacgccctgtgcttcagccctaaccgctactggctgtgtgctgccacaggccccagcatcaagatctgggatttagagggaaagatcattgtagatgaactgaagcaagaagttatcagtaccagcagcaaggcagaaccaccccagtgcacctccctggcctggtctgctgatggccagactctgtttgctggctacacggacaacctggtgcgagtgtggcaggtgaccattggcacacgctagaagt

SEQ ID NO: 34 - protein, 271 aa, Homo sapiens
MRPTMEFHSVLCGVTPTLLVMWLSPQMASLPSQAPGMEPCASGISQRKGTTTRRFVGHTKDVLSVAFSSDNRQIVSGSRDKTIKLWNTLGVCKYTVQDESHSEWVSCVRFSPNSSNPIIVSCGWDKLVKVWNLANCKLKTNHIGHTGYLNTVTVSPDGSLCASGGKDGQAMLWDLNEGKHLYTLDGGDIINALCFSPNRYWLCAATGPSIKIWDLEGKIIVDELKQEVISTSSKAEPPQCTSLAWSADGQTLFAGYTDNLVRVWQVTIGTR

SEQ ID NO: 35 - DNA, 16 bp, Artificial Sequence
  H-T11C primer
aagctttttttttttc

SEQ ID NO: 36 - DNA, 13 bp, Artificial Sequence
  H-AP15 primer
aagcttacgcaac

SEQ ID NO: 37 - DNA, 626 bp, Homo sapiens
gcccgaacatggactccgccggccaagatatcaacctgaattctcctaacaaaggtctgctgcctgactccatgacggatgttcctgtcgacacaggtgtggctgcccggactcctgctgttgagggtctgacagaggctgaggaggaggagctcagggctgagcttaccaaggtggaagaggaaattgtcactctgcgccaggtcctggcagccaaggagaggcactgtggagagctcaagaggaggctgggcctctccaccctgggggagctaaaacagaacctgtccaggagctggcatgacgtgcaggtctctagcgcctatgtgaaaacttctgagaaacttggagagtgaatgagaaagtgacccagtcagacctctacaagaagactcaggagactctttcacaggcaggacagaagacttcagctgccctgtccacagtgggctctgccatcagcaggaagcttggagacatgaggaactctgcgaccttcaagtcgtttgaggaccgagttgggaccataaagtctaaggttgtgggtgacagagggaacggcagtgacaacctcccttcctcagcggggagtggtgacaagcccctgtcggatcccgcacctttct

SEQ ID NO: 38 - protein, 115 aa, Homo sapiens [sequence omitted]

SEQ ID NO: 39 - DNA, 2302 bp, Homo sapiens
ttttttttttttttttttttttttttttttttttttttaaagttcagctttttattgaacatgttataaaagaggtttagtcaaaaagaccaaagcccatgtcatcatcagactcctcggattcttctttctttgcttccactttcttctcctcagctggagcagcagcagtggagggggcaggacctcctgctggtgcagcaccagctgctggagcaggtccaccggcccctacattgcagatgaggctcccaatgttgacgttggccagggcctttgcaaacaagccaggccaaaaaggctcaacatttacaccggctgctttaatgagggcattgatcttatcctccgtgactgtcacctcatcgtcgtgcagaatgagggccgagtagatgcaggcgagctcggagacagaggccatggcgcgggcgagtgtagggctggcgctgccggacgcggtgctagtcgccggatgaagtgagggcctcaccccaacgcagccttagcttcctcggaaggaccgagcaccttgggaaaagaaaccaacagttgaagagaaggcaaaagcagatacgttaaaacttccacctacatttttttgtggagtctgtagtgatactgatgaagacaatggaaatggggaagactttcaatcagagcttcaaaaagttcaggaagctcaaaaatctcagacagaagaaataactagcacaactgacagtgtatatacaggtgggactgaagtgatggtaccttctttctgtaaatctgaagaacctgattctattaccaaatccgttagttcaccatctgtttcctctgaaactatggacaaacctgtagatttgtcaactagaaaggaaattgatacagattctacaagccaaggggaaagcaagatagtttcatttggatttggaagtagcacagggctctcatttgcagacttggcttccagtaattctggagattttgcttttggttctaaagataaaaatttccaatgggcaaatactggagcagctgtgtttggaacacagtcagtcggaacccagtcagccggtaaagttggtgaagatgaagatggtagtgatgaagaagtagttcataatgaagatatccattttgaaccaatagtgtcactaccagaggtagaagtaaaatctggagaagaagatgaagaaattttgtttaaagagagagccaaactttatagatgggatcgggatgtcagtcagtggaaggagcgcggtgttggagatataaagattctttggcatacaatgaagaattattaccggatcctaatgagaagagaccaggtttttaaagtgtgtgcaaaccacgttattactaaaacaatggaattaaagcccttaaatgtttcaaataatgctttagtttggactgcctcagattatgctgatggagaagcaaaagtagaacagcttgcagtgagatttaaaactaaagaagtagctgattgtttcaagaaaacatttgaagaatgtcagcagaatttaatgaaactccagaaaggacatgtatcactggcagcagaattatcaaaggagaccaatcctgtggtgttttttgatgtttgtgcggacggtgaacctctagggcggataactatggaattattttcaaacattgttcctcggactgctgagaacttcagagcactatgcactggagagaaaggctttggtttcaagaattccatttttcacagagtaattccagattttgtttgccaaggaggagatatcaccaaacatgatggaacaggcggacagtccatttatggagacaaatttgaagatgaaaattttgatgtgaaacatactggtcctggtttactatccatggccaatcaaggccagaataccaataattctcaatttgttataacactgaagaaagcagaacatttggactttaagcatgtagtatttgggtttgttaaggatggcatggatactgtgaaaaagattgaatcatttggttctcccaaagggtctgtttgtcgaagaataactatcacagaatgtggacagatataaaatcattgttgttcatagaaaatttcatctgtataagcagttggattgaagcttagctattacaatttgatagttatgttcagcttttgaaaatggacgtttccgatttacaaatgtaaaattgcagcttatagctgttgtcactttttaatgtgttataattgaccttgcatggtgtgaaataaaagtttaaacactggtgtaaaaaaaaaaaaaaaaaaaaaaaaaaaaaaaaa

SEQ ID NO: 40 - protein, 439 aa, Homo sapiens [sequence omitted]

SEQ ID NO: 41 - DNA, 16 bp, Artificial Sequence
  H-T11A primer
aagcttttttttttta

SEQ ID NO: 42 - DNA, 13 bp, Artificial Sequence
  H-AP32 primer
aagcttcctgcaa